(19) United States
(12) Patent Application Publication
(10) Pub. No.: US 2009/0117089 A1
(43) Pub. Date: May 7, 2009

(54) GLYCOLIPIDS AND ANALOGUES THEREOF AS ANTIGENS FOR NK T CELLS

(75) Inventors: Ralph M. Steinman, Westport, CT (US); Kang Liu, New York, NY (US); Moriya Tsuji, New York, NY (US)

Correspondence Address: FOX ROTHSCHILD LLP;PRINCETON PIKE CORPORATE CENTER, 2000 Market Street, Tenth Floor, Philadelphia, PA, 19103, US

(73) Assignee: The Rockefeller University, New York, NY (US)

(21) Appl. No.: 12/067,442

(22) PCT Filed: Sep. 18, 2006
(86) PCT No.: PCT/US06/36454

§ 371 Date: Jul. 30, 2008

Related U.S. Application Data
(60) Provisional application No. 60/717,754, filed on Sep. 19, 2005.
(60) Provisional application No. 60/836,111, filed on Aug. 8, 2006.
(60) Provisional application No. 60/841,278, filed on Aug. 31, 2006.

(51) Int. Cl. A61K 35/12; A61P 35/00

(52) U.S. Cl. 424/93.71; 424/93.7

(57) ABSTRACT

This invention relates to immunogenic compounds which may serve as ligands for NKT (natural killer T) cells and to methods of use thereof in modulating immune responses.

### STATEMENT AS TO FEDERALLY SPONSORED RESEARCH

This invention was conducted with United States Government support under National Institutes of Health Grant Numbers AI 13013 and AI 51573. The government has certain rights in the invention.

### FIELD OF THE INVENTION

This invention relates to immunogenic compounds which may serve as ligands for NKT (natural killer T) cells and to methods of use thereof in modulating immune responses.

### BACKGROUND OF THE INVENTION

CD1 molecules are a family of highly conserved antigen presenting proteins that are similar in function to classical MHC molecules. While classical MHC molecules present peptides, CD1 proteins bind and display a variety of lipids and glycolipids to T lymphocytes. The five known isoforms are classified into two groups, group I (CD1a, CD1b, CD1c, and CD1e in humans) and group II (CD1d in humans and mice) based on similarities between nucleotide and amino acid sequences.

A great diversity of lipids and glycolipids has been shown to bind specifically to each of the four isoforms. The first crystal structure of murine (m)CD1d revealed that it adopts a folded conformation closely related to major histocompatibility complex (NHC) class I proteins. It further revealed that mCD1d could accommodate long lipid tails in two hydrophobic pockets, designated A′ and F′, located in the binding groove. Two additional structures of hCD1b and hCD1a confirmed this model by demonstrating that CD1, when loaded with antigenic glycolipids, binds the lipid portion in a hydrophobic groove while making available the hydrophilic sugar moiety to make contact with the T-cell receptor.

Mammalian and mycobacterial lipids are known to be presented by human CD1a, CD1b, CD1c and CD1d [Porcelli, S. A. & Modlin, R. L. (1999) Annu. Rev. Immunol. 17, 297-329] α-GalCer, a lipid found in the marine sponge Agelas mauritianus, has been, to date, the most extensively studied ligand for CD1d. α-GalCer, when bound to CD1d, stimulates rapid Th1 and Th2 cytokine production by Vα14i natural killer T (Vα14i NKT) cells in mice and the human homologue Vα24i NKT cells. However, its physiological significance in mammals remains unclear as it is enigmatic why an α-galactosyl ceramide of marine origin is such a potent agonist. Other known ligands, such as a bacterial phospholipid (PIM₄), a tumor derived ganglioside GD3, a C-linked analog of α-GalCer, α-GalCer analogs with different lipid chain lengths and a phosphoethanolamine, found in human tumor extracts, stimulate only relatively small populations of CD1d-restricted NKT cells.

Natural Killer (NK) cells typically comprise approximately 10 to 15% of the mononuclear cell fraction in normal peripheral blood. Historically, NK cells were first identified by their ability to lyse certain tumor cells without prior immunization or activation. NK cells also serve a critical role in cytokine production, which may be involved in controlling cancer, infection and possibly in fetal implantation.

### SUMMARY OF THE INVENTION

This invention provides, in one embodiment, a method for treating, delaying onset of, reducing incidence of, suppressing or reducing the severity of neoplasia in a subject, comprising the steps of:

a. culturing immature dendritic cells with a neoplastic cell;

b. contacting the culture in (a) with a compound characterized by the structure of the formula I:

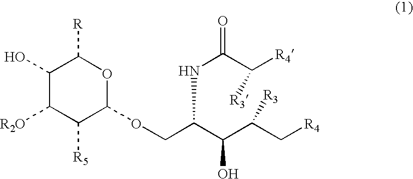

wherein,
R═COOR₁ or CH₂OR₁;
R₁═H or an alkyl group;
R₂═H or SO₃⁻;
R₃═H or OH;
R₃′═H or OH;
R₄═H, unsaturated or saturated, alkyl group;
R₄′═H, unsaturated or saturated, alkyl group; and
R₅═OH, acetamido or a halogen atom;
or a pharmaceutically acceptable salt thereof,
wherein if R═CH₂OR₁, R₂═H, R₃ is OH and R₃′ is H, then R₅=acetamido, halogen atom or OH in an axial position or R₄═H, unsaturated or saturated, alkyl chain having 9 carbon atoms or fewer, or R₄′═H, unsaturated or saturated, alkyl chain having 20 carbon atoms or fewer; and

c. administering the culture in (b) to said subject.

In one embodiment, the compound is represented by the structure of formula 2:

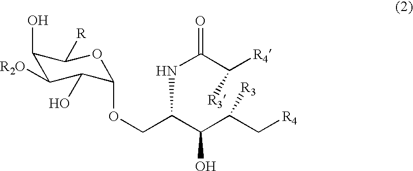

wherein
   R═COOR₁ or CH₂OR₁;
   R₁═H or an alkyl group;
   R₂═H or SO₃⁻;
   R₃═H or OH;
   R₃′═H or OH; and
   R₄═H, unsaturated or saturated, alkyl group; and
   R₄′═H, unsaturated or saturated, alkyl group;
   or a pharmaceutically acceptable salt thereof,
   wherein if R═CH₂OR₁, R₂═H, R₃ is OH and R₃′ is H, then R₄═H, unsaturated or saturated, alkyl chain having 9 carbon atoms or fewer, or R₄′═H, unsaturated or saturated, alkyl chain having 20 carbon atoms or fewer.

In another embodiment, the compound is represented by the structure of formula 3:

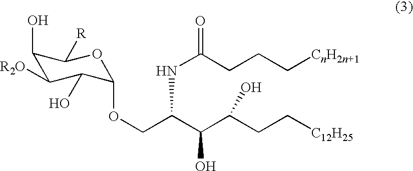

wherein,
R═COOR₁ or CH₂OR₁;
R₁═H or an alkyl group;
R₂═SO₃⁻; and
n=integer;
or a pharmaceutically acceptable salt thereof.

In another embodiment, the compound is represented by the structure of formula 4:

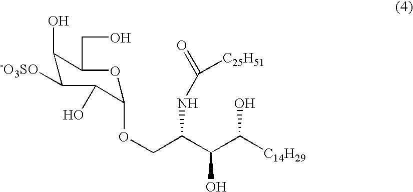

or a pharmaceutically acceptable salt thereof.

In another embodiment, the salt may be, inter alia, a sodium salt.

In another embodiment, the compound is represented by the structure of formula 5:

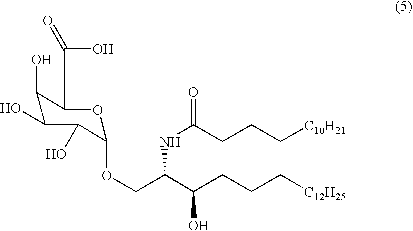

In another embodiment, the compound is represented by the structure of formula 6:

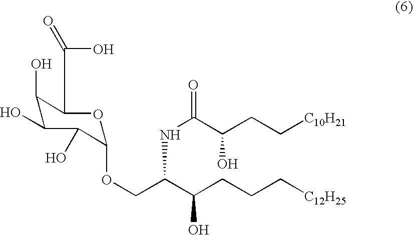

In another embodiment, the compound is represented by the structure of formula 7:

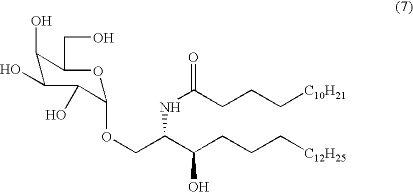

In another embodiment, the compound is represented by the structure of formula 8:

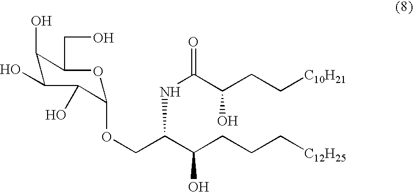

In one embodiment, the compound is represented by the structure of formula 9:

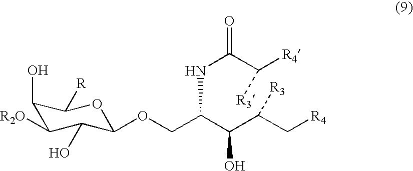

wherein,
R═COOR₁ or CH₂OR₁
R₁═H or an alkyl group;
R₂═H or SO₃⁻;
R₃═OH;
R₃′═H or OH; and
R₄═H, unsaturated or saturated, alkyl group; and
R₄′═H, unsaturated or saturated, alkyl group;
   or a pharmaceutically acceptable salt thereof,
wherein if R═CH₂OR₁, R₂═H, R₃ is OH and R₃′ is H, then R₄═H, unsaturated or saturated, alkyl chain having 9 carbon atoms or fewer, or R₄═H, unsaturated or saturated, alkyl chain having 20 carbon atoms or fewer.

In another embodiment, the compound is represented by the structure of formula 10:

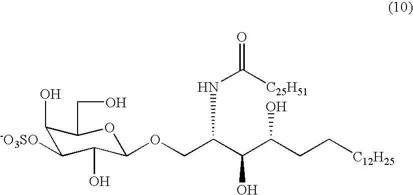

or a pharmaceutically acceptable salt thereof. In another embodiment, the salt may be, inter alia, a sodium salt.

This invention provides, in one embodiment, a method for treating, delaying onset of, reducing incidence of, suppressing or reducing the severity of neoplasia in a subject, comprising the steps of:

a. culturing immature dendritic cells with a neoplastic cell;

b. contacting the culture in (a) with a compound characterized by the structure of the formula 11:

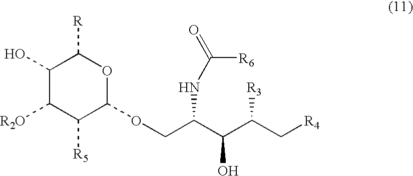

wherein,
   R═COOR₁ or CH₂OR₁;
   R₁═H or an alkyl group;
   R₂═H or SO₃⁻;
   R₃═H or OH;
   R₄═H, unsaturated or saturated, alkyl group;
   R₅═OH, acetamido or a halogen atom; and
   R₆═X-A
   A=
   dialkyl phenyl;

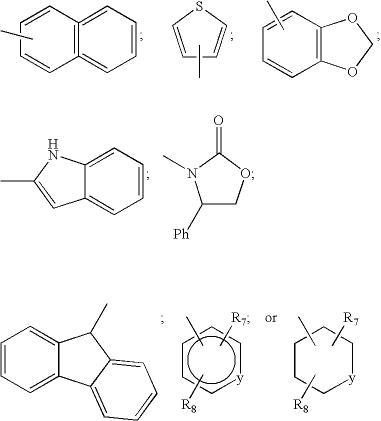

X=alkyl, alkenyl, alkoxy, thioalkoxy, substituted furan, or unsubstituted furan;
Y═N or C
R7=halogen, H, phenyl, alkyl, alkoxy, nitro or CF3; and
R8=methyl or H;
   or a pharmaceutically acceptable salt thereof; and

c. administering the culture in (b) to said subject.

In one embodiment, the compound is represented by the structure of formula 12:

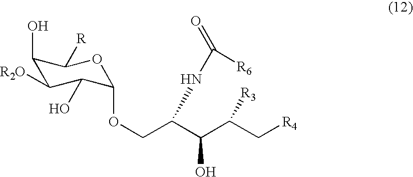

or a pharmaceutically acceptable salt thereof.

In another embodiment, the compound is represented by the structure of formula 3:

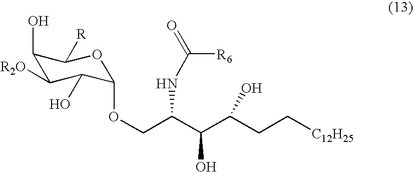

or a pharmaceutically acceptable salt thereof.

In another embodiment, the compound is represented by the structure of formula 4:

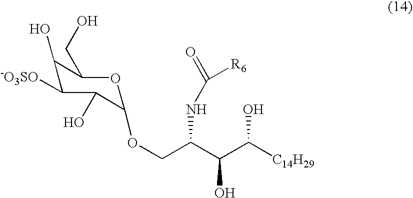

or a pharmaceutically acceptable salt thereof.

In another embodiment, the salt may be, inter alia, a sodium salt.

In another embodiment, the compound is represented by the structure of formula 15:

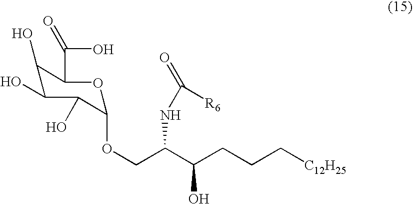

In another embodiment, the compound is represented by the structure of formula 16:

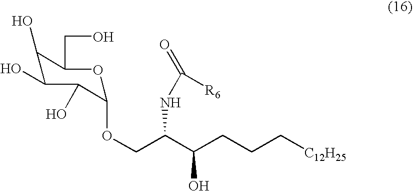

In one embodiment, the compound is a ligand for an NKT (natural killer T) cell and is in a complex with a CD1 molecule. In one embodiment, the neoplastic cell is irradiated. In one embodiment, the compound is at a concentration ranging from 1-1000 ng/ml. In one embodiment, the culture is administered to the subject intravenously. In one embodiment, the immature dendritic cells are isolated from said subject having or predisposed to having neoplasia. In one embodiment, the immature dendritic cells are isolated from the blood or bone marrow of said subject. In one embodiment, the immature dendritic cells are syngeneic or autologous with respect to said subject. In one embodiment, the culture in step (c) comprises dendritic cells with MHC II^{hi}, CD80^{hi}, CD86^{hi}, B7-H1^{hi}, B7-DC^{hi}, or a combination thereof. In one embodiment, the immature dendritic cells express a CD1d molecule. In one embodiment, the neoplastic cell is isolated from said subject. In one embodiment, the subject has preneoplastic or hyperplastic cells or tissue. In one embodiment, the culture further comprises NKT cells.

In another embodiment, the invention provides a method for treating, delaying onset of, reducing incidence of, suppressing or reducing the severity of neoplasia in a subject, comprising the step of administering to said subject a composition comprising a neoplastic cell and a compound as herein described.

In another embodiment, this invention provides a composition comprising an immature dendritic cell, a hyperplastic, preneoplastic or neoplastic cell and a compound as herein described. In another embodiment, this invention provides a composition comprising a hyperplastic, preneoplastic or neoplastic cell and a compound as herein described.

### BRIEF DESCRIPTION OF THE DRAWINGS

FIG. 1 demonstrates structures of α-galactosylceramide, sulfatide and 3-O-sulfo-α/β-galactosylceramides 10, 24, according to embodiments of the invention.

FIG. 2 demonstrates the preparation of (a) compound IV, according to embodiments of the invention; (b) the preparation of compound 4, according to embodiments of the invention.

FIG. 3 demonstrates the preparation of compound XV, according to embodiments of the invention.

FIG. 4 demonstrates (a) the preparation of compound XVIII, according to embodiments of the invention; (b) the preparation of compound 10, according to embodiments of the invention.

FIG. 5 depicts structures of glycolipids and analogs thereof, according to embodiments of the invention.

FIG. 6 schematically depicts the synthesis of sphingosine, according to embodiments of the invention, as conducted herein. Reagents and conditions; a) C2H3MgBr, THF, Anti:Syn 3.5:1 61%; b) (i) Grubbs catalyst 2nd generation, CH2Cl2, Pentadecene, 71%; (ii) BzCl, pyridine, 90%; (iii) Amberlyst 15 H+ form, MeOH 70%.

FIG. 7 schematically depicts the synthesis of some glycolipids, according to embodiments of the invention, as conducted herein. Reagents and conditions; a) 32, TMSOTf, 67%; b) (i) TFA, DCM, (ii) HBTU, myristic acid or 2-(S)-hydroxy myristic acid, n-Morpholine, 92% 2 steps; c) H₂, 20% Pd(OH)2, (ii) LiOH, H2O:THF:MeOH, 38%, 2 steps; d) 36, TMSOTf, 60%; e) (i) TFA, DCM, (ii) HBTU, myristic acid or 2-(S)-hydroxy myristic acid, n-Morpholine, ≈92% 2 steps; f) (i) NaOMe, MeOH, (ii) Pd/C, H₂, EtOH, 90% 2 steps; g) 40, TMSOTf, 62%. h) (i) TFA, DCM, (ii) HBTU, nervonic acid, n-Morpholine, 60% 2 steps; i) (i) NaOMe, MeOH, quant. (ii) Bu₂SnO, MeOH, (iii) Me₃N.SO₃, THF, 95%; j-k) (i) LDA, TMSOOTMS, (ii) H+, MeOH, 30%, 2 steps; then LiOH, H₂O:MeOH:THF, 81%; 1) Novozyme 435, CH₂═CHOAc, 54% based on S isomer.

FIG. 8 demonstrates the IL-2 secretion profiles obtained with glycolipids, according to embodiments of the invention. (a) IL-2 secretion profiles obtained with 3-O-sulfo-GalCer, as compared to α-GalCer and analogues. (b) Dose dependent secretion of IL-2 by Sphingomonas GSLs and analogues.

FIG. 9 demonstrates human NKT cell responses to glycolipids, according to embodiments of the invention. Human Vα24i NKT cells response to synthetic Sphingomonas and sulfatide glycolipids, in terms of IFN-γ (a) and IL-4 (b) release after culture with 4×10⁵ autologous immature CD14+ dendritic cells pulsed with the indicated glycolipid antigens at 10 μg/ml. Negative controls included similar numbers of NKT cells and dendritic cells, cultured without added glycolipid. Data represent mean±S.D. of duplicate well; (c) in vitro INF-γ secretion by human CD161⁺ NK+NKT cells (2×10⁵/well) in the presence of CD14⁺ DCs (4×10⁵/well) and 20 μg/ml of various glycolipids; (d) in vitro IL-4 secretion by human CD161⁺ NK+NKT cells (2×10⁵/well) in the presence of CD14⁺ DCs (4×10⁵/well) and 20 μg/ml of various glycolipids.

FIG. 10 demonstrates a flow cytometric analysis of a human Vα24i human NKT cell line with human CD1d dimers that were unloaded or loaded with 10M of the indicated glycolipid antigen, according to embodiments of the invention. The cells were also stained with anti-human CD3-PerCP.

FIG. 11 depicts a computer-generated model of GSL-1 docked to the crystal structure of mCD1d, according to embodiments of the invention. The two acyl tails fit nicely into the hydrophobic pockets of the protein allowing for the sugar head group to be presented for NKT cell recognition.

FIG. 12 demonstrates IL-2 secretion profiles obtained from murine NKT cells presented with the glycolipids as indicated, according to embodiments of the invention.

FIG. 13 demonstrates IL-2 secretion profiles obtained from murine NKT cells presented with other glycolipids as indicated, according to embodiments of the invention.

FIGS. 14, 15 and 16 demonstrate IFN-γ secretion from human NKT cells presented with the glycolipids as indicated, supplied at the indicated concentration.

FIGS. 17 and 18 demonstrate IFN-γ secretion from human NKT cells presented with the glycolipids as indicated, supplied at higher concentration.

FIG. 19 demonstrates similar IFN-γ secretion from human NKT cells presented with the glycolipids, at the indicated concentration, in the context of Hela cells transfected with CD1d.

FIG. 20 demonstrates IL-4 secretion from human NKT cells presented with the glycolipids, at the indicated concentration in the context of dendritic cells (A) or transfected Hela cells (B).

FIG. 21 depicts the superimposition of docking results of compound 84 from Example 10 (yellow) with the crystal structure of α-GalCer (green)/hCD1d complex. The α2 helix is removed for clarity. The overall binding motif of the docked compound did not notably deviate from the crystallized structure. The terminal phenyl group 84 is within distance to interact with the aromatic ring of Tyr73.

FIG. 22 demonstrates intravenous delivery of dying tumor cells to spleen CD11c+ DCs in vivo. Mice were injected i.v. with 2×10⁷ dying, irradiated, CFSE-labeled A20 tumor cells and 2 hrs later, the spleens were taken for sectioning and staining for the injected A20 tumor cells and for CD11c (anti-mouse CD11c-PE), to observe selective uptake of dying A20 tumor cells by CD11c+ splenic DCs.

FIG. 23 demonstrates intravenous delivery of dying tumor cells to CD8+ CD11c+ DCs in vivo. (a) Kinetics of uptake of 20×10⁶ dying, irradiated, CFSE-labeled J558− tumor cells, injected i.v. or s.c., by CD11c+ spleen (SPLN), draining (LN) and distal (dLN) lymph node DCs, analyzed with flow cytometry as in (b). (b) Flow cytometric assays to show selective uptake of irradiated CFSE-labeled J558 tumor cells by CD8α, CD11c+ splenic DCs 2 hrs later.

FIG. 24 demonstrates that α-Gal Cer injection rapidly matures CD1d-rich, splenic DCs capturing dying cells. Balb/c mice were injected with PBS or 20×10⁶ irradiated CFSE-labeled J558-tumor cells i.v. in the presence or absence of 2 μg α-Gal Cer. 4.5 hrs later, bulk spleen cells were prepared from the mice and stained with CD11c-APC and PE conjugated mAbs CD40, 80, 86, B7-H1, B7-DC, Ld, I-Ad and CD1d. The data are shown for the CD11c+total DC population (top 3 rows) and CD11c+ CFSE+ phagocytic population (bottom two rows).

FIG. 25 demonstrates the acquired resistance to tumor cells after vaccination with the tumor in conjunction with α-Gal Cer. (a) Mice were vaccinated with PBS, 2 μg α-Gal Cer, 20×10⁶ irradiated MHC I-ve, J558 Ld⁻ cells with or without 2 μg α-Gal Cer i.v. One week later, mice were challenged with a lethal tumorogenic dose of 5×10⁶ live MHC I⁺ J558 s.c. (b) Mice were vaccinated with PBS, 2 μg α-Gal Cer, 5×10⁶ irradiated A20 cells with or without 2 μg α-Gal Cer i.v. Two weeks later, mice were challenged with a lethal tumorogenic dose of 5×10⁶ live A20 s.c. The mice were monitored every other day for tumor growth and were scored positive when the tumors were palpable (n=5). (c) Mice were vaccinated with 20×10⁶ irradiated J558− cells plus 2 μg α-Gal Cer i.v., and one week later, challenged with 5×10⁶ J558 or 5×10⁶ Meth A s.c. (d) Mice were vaccinated with 5×10⁶ irradiated J558− cells and either 2 μg α-Gal Cer i.v., 50 μg αCD40 i.p, or 50 μg PolyI:C i.p., and 3 wks later, challenged with 5×10⁶ J558 s.c. (e) 8 wks after vaccination, mice were challenged with a lethal dose of 5×10⁶ live J558 tumor cells. (f) Mice were vaccinated with PBS, 20×10⁶ irradiated J558− cells alone, or 20×10⁶ irradiated J558− cells with 2 μg αGal Cer i.v. or s.c., and one week later, challenged with 5×10⁶ live MHC class I positive J558 s.c. (g) To detect a therapeutic effect, mice were injected with 1 or 5×10⁶ live J558 tumor cells s.c., and 3 days later treated with PBS, or 20×10⁶ irradiated J558− cells with 2 μg α-Gal Cer i.v. The mice were monitored every other day for tumor growth and were scored positive when the tumors were palpable. Each group included at least 5 mice, and one representative experiment of three is shown.

FIG. 26 demonstrates CD4⁺ or CD8⁺ T cell depletion abrogates tumor immunity at the time of challenge. (a) Mice were vaccinated with 20×10⁶ irradiated J558⁻ tumor cells plus α-Gal Cer. 7 days after immunization, mice were challenged with 5×10⁶ MHC Class I⁺ or MHC Class I⁻ J558 cells s.c. (b) Wild type Balb/c mice or J□18−/− mice were vaccinated with 20×10⁶ irradiated J558− tumor cells plus α-Gal Cer, 3 wks after vaccination, the mice were challenged with 5×10⁶ J558 cells s.c. (c) 8 wks after vaccination with 20×10⁶ irradiated J558− tumor cells and α-Gal Cer, mice received 1 mg of anti-CD4, anti-CD8 or control rat IgG. One day later, all mice were challenged with 5×10⁶ J558 and monitored every other day for tumor growth. Mice were scored positive when the tumors were palpable. Each group included 5 mice; one representative experiment of two is shown.

FIG. 27 demonstrates the proliferation and differentiation of tumor antigen-specific T cells in response to dying tumor cell delivery in vivo. (a) 3×10⁶ CFSE-labeled, naïve CD8⁺ P1CTL T cells were injected i.v. into Balb/c mice. 1 d later 20×10⁶ J558− cells were injected i.v. with or without 2 μg α-Gal Cer coinjection. 3 d later, mice were sacrificed and spleen cells were stained with CD8-APC and CD25 or CD62L-PE. (b) Spleen cells obtained as in (a) were cultured at 10⁷/ml with 1 μg/ml P1A 35-43 peptide (LPYLGWLVF) for 4 h at 37° C. in the presence of 5 μg/ml BFA. The cells were stained for CD8, and intracellular IFN-γ and IL-2 were identified with PE-conjugated mAb after fixation and permeabilization. Black lines are from mice immunized with J558− cells alone, and grey line from mice immunized with J558⁻ cells plus α-Gal Cer.

FIG. 28 demonstrates that maturing DCs mediate P1A antigen presentation and elicit tumor immunity in vivo. Mice were given α-Gal Cer or PBS together with irradiated J558⁻ cells. 4 h later, CD11c⁺ and CD11c⁻ cells were isolated and used to stimulate CD8⁺ TCR transgenic T cells from P1CTL mice in vitro (a) or in vivo (b). In (a), in vitro T cell proliferation was measured by a ³H thymidine pulse at 40-50 h. In (b), in vivo proliferation of CFSE-labeled P1CTL T cells was measured 3 days later. (c) Splenic CD11c⁺ and CD11c⁻ cells were isolated from mice injected with irradiated J558⁻ cells and α-Gal Cer 4 hrs earlier. 1×10⁶ CD11c⁺ or 5×10⁶ CD11c− cells were then transferred i.v. into naïve Balb/c mice. 1 wk later the mice were challenged with live 5×10⁶ J558 cells. Mice were monitored every 3 days for tumor growth. Each group includes 12-17 mice pooled from three experiments.

FIG. 29 shows maturation of DC surface markers with αGalCer, 24 and 27, as well as other stimuli in vivo. (A) Structures of the synthetic CD1d binding glycolipid αGalCer, and analogues 24 (3-O-sulfo-alpha-galactosylceramide) and 27 (sphingosine-truncated (C9)). (B) 15 h after i.v. administration of αGalCer (2 μg), 24 (2 μg or 0.2 μg), 27 (2 μg or 0.2 μg) or PBS to BALB/C mice, spleen DCs were isolated using anti-CD11c-coated magnetic beads (purity >95%±2%) and stained with FITC-CD11c and APC-CD8α (to identify DCs and their CD8⁺ and CD8⁻ subsets) and with PE-labeled mAbs to CD40, CD80, CD86, MHC II or I-A^{d}, CD119, B7-DC or PD-L2, anti-B7-H1 or PD-L1 (black tracings) and their respective PE-labeled isotype controls (grey tracing). (C) As in (B) but αCD40 (25 μg) and PolyIC (50 μg) were compared to αGalCer and 24 as DC maturation stimuli. (D) As in (B) and (C), but different doses of 24 and αGalCer were examined (2, 0.2, 0.02 and 0.002 μg/mouse). The degree of DC maturation induced, correlated with the dose of glycolipid administered (data shown are for CD11c⁺CD8⁺ DCs only). Results are representative of three independent experiments.

FIG. 30 shows DC maturation by αGalCer and 24 in vivo requires NKT cells. (A) Maturation was assessed by increased CD80 and CD86 expression in Jα18^{-/-} mice (lacking NKT cells) exposed to αGalCer or 97A, or with αCD40 as a positive control. (B) Serum concentrations of IFN-γ, IL-12p70, IL-10, IL-5 and TNF-α in C57B1/6 and Jα18^{-/-} mice given 2 μg of αGalCer, 2 μg of 97A or PBS i.v. Data are means obtained from two mice in two experiments. (C) 15 h after i.v. administration of either αGalCer (2 μg), 24 (2 μg), irradiated J558Ld− tumor cells (7.5×10⁶), or PBS, spleen DCs were isolated using anti-CD11c-coated magnetic beads (purity >95%±2%). Graded numbers of DCs from BALB/C mice were added to 2.5×10⁵ syngeneic BALB/C (left panel) or allogeneic C57B1/6 (right panel) T cells, for 3 days in flat bottomed 96-well plates. Proliferative responses were measured by ³[H]-thymidine incorporation.

FIG. 31 shows Responses to αGalCer vs 24. (A) Cytokine release into the serum induced by 24 and αGalCer (2 μg) i.v. Serum concentrations of IFN-γ, IL-12p70, IL-4, IL-2 and TNF-α were evaluated at 2, 6, 12 and 24 h using the Luminex assay. (B) Mice were immunized with different doses of αGalCer or 24 (2 μg to 0.002 μg) i.v., and 15 h later, serum concentrations of IFN-γ and IL-12p70 were evaluated. Data are means obtained from three experiments.

FIG. 32 shows cytokine production by splenic DCs and NKT cells after i.v. injection of αGalCer or 24. Mice were stimulated 2 h, 6 h and 12 h in vivo with either αGalCer or 24 (2 μg or 0.2 μg). Spleen CD11c+positive cells were isolated and cultured for 4 hours in the presence of BFA (1 μg/ml). We then measured IFN-γ and IL-12 production by CD11c+DX5− DCs as well as CD11c+DX5+ NKT cells by intracellular staining. Production of IFN-γ by CD11c+DX5+ NKT cells and production of IL-12 by CD11c+DX5− DCs were significantly higher in mice treated with 97A vs. αGalCer. Representative results of one of three experiments are shown.

FIG. 33 shows 24 induces long-lived, prophylactic tumor immunity, when co-administered with irradiated J558Ld− tumor cells. (A) Mice were vaccinated with 2 or 0.2 μg of 97A or αGalCer, with or without 5×10⁶ irradiated MHC Class I⁻ J558 tumor cells i.v. Then 2 wks later, mice were challenged with a tumorgenic dose of 5×10⁶ live MHC Class I⁺ J558 cells s.c. (B) Mice were vaccinated with 5×10⁶ irradiated J558⁻ cells and either 2 μg αGalCer or 97A i.v., 50 μg αCD40 i.p, 50 μg polyIC i.p., or both αCD40 and polyIC and, 3 wk later, each group was challenged with 5×10⁶ J558 s.c.

FIG. 34 shows 24 binds CD1d molecule and efficiently expands human NKT cells. Binding of CD1d dimers loaded with synthetic αGalCer analogs to invariant T cell receptor NKT cells. NKT cells were gated based on the expression of Vα24/Vβ11 and evaluated for binding of CD1d dimer. Human mature monocyte derived DC were loaded with α-GalCer or 24 and cultured with CD14 negative responder cells at a DC to responder ratio of 1:10. NKT expansion was monitored by flow cytometry based on the expression of invariant T cell receptor (Vα24/Vβ11).

### DETAILED DESCRIPTION OF THE PRESENT INVENTION

This invention provides, in one embodiment, a method for treating, delaying onset of, reducing incidence of, suppressing or reducing the severity of neoplasia in a subject, comprising the steps of:

a. culturing immature dendritic cells with a neoplastic cell;

b. contacting the culture in (a) with a compound characterized by the structure of the formula I:

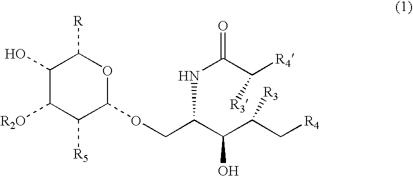

wherein,
R═COOR₁ or CH₂OR₁;
R₁═H or an alkyl group;
R₂═H or SO₃⁻;
R₃═H or OH;
R₃′═H or OH;
R₄═H, unsaturated or saturated, alkyl group;
R₄′═H, unsaturated or saturated, alkyl group; and
R₅═OH, acetamido or a halogen atom;
or a pharmaceutically acceptable salt thereof,
   wherein if R═CH₂OR₁, R₂═H, R₃ is OH and R₃′ is H, then R₅=acetamido, halogen atom or OH in an axial position or R₄═H, unsaturated or saturated, alkyl chain having 9 carbon atoms or fewer, or R₄′═H, unsaturated or saturated, alkyl chain having 20 carbon atoms or fewer; and

d. administering the culture in (b) to said subject.

In one embodiment, the compound is represented by the structure of formula 2:

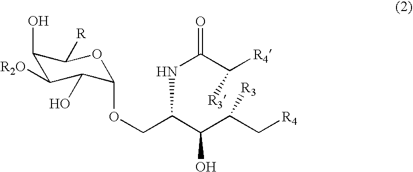

wherein
   R═COOR₁ or CH₂OR₁;
   R₁═H or an alkyl group;
   R₂═H or SO₃⁻;
   R₃═H or OH;
   R₃′═H or OH; and
   R₄═H, unsaturated or saturated, alkyl group; and
   R₄′═H, unsaturated or saturated, alkyl group;
   or a pharmaceutically acceptable salt thereof,
      wherein if R═CH₂OR₁, R₂═H, R₃ is OH and R₃′ is H, then R₄═H, unsaturated or saturated, alkyl chain having 9 carbon atoms or fewer, or R₄′═H, unsaturated or saturated, alkyl chain having 20 carbon atoms or fewer.

In another embodiment, the compound is represented by the structure of formula 3:

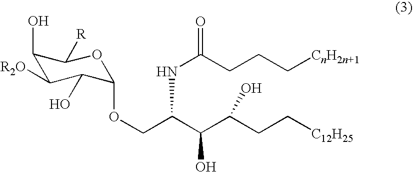

wherein,
R═COOR₁ or CH₂OR₁;
R₁═H or an alkyl group;
R₂═SO₃⁻; and
n=integer;
or a pharmaceutically acceptable salt thereof.

In another embodiment, the compound is represented by the structure of formula 4:

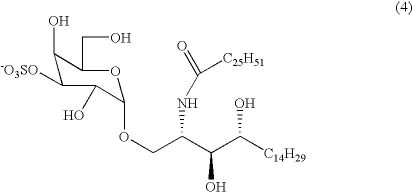

or a pharmaceutically acceptable salt thereof.

In another embodiment, the salt may be, inter alia, a sodium salt.

In another embodiment, the compound is represented by the structure of formula 5:

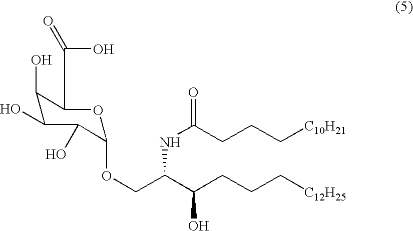

In another embodiment, the compound is represented by the structure of formula 6:

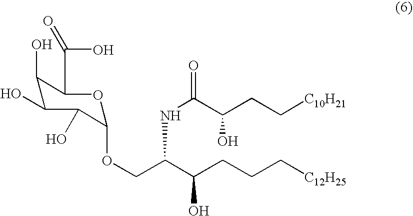

In another embodiment, the compound is represented by the structure of formula 7:

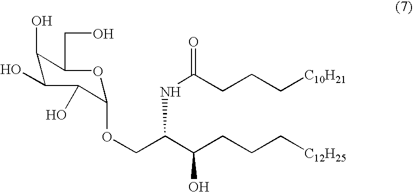

In another embodiment, the compound is represented by the structure of formula 8:

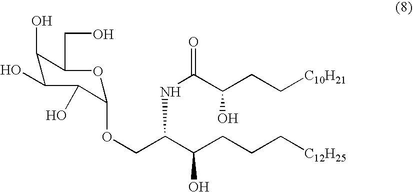

In one embodiment, the compound is represented by the structure of formula 9:

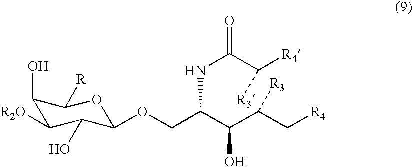

wherein,
R═COOR₁ or CH₂OR₁
R₁═H or an alkyl group;
R₂═H or SO₃⁻;
R₃═OH;
R₃′═H or OH; and
R₄═H, unsaturated or saturated, alkyl group; and
R₄′═H, unsaturated or saturated, alkyl group;
   or a pharmaceutically acceptable salt thereof,
wherein if R═CH₂OR₁, R₂═H, R₃ is OH and R₃′ is H, then R₄═H, unsaturated or saturated, alkyl chain having 9 carbon atoms or fewer, or R₄′═H, unsaturated or saturated, alkyl chain having 20 carbon atoms or fewer.

In another embodiment, the compound is represented by the structure of formula 10:

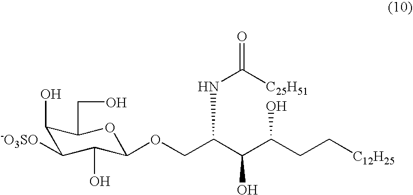

or a pharmaceutically acceptable salt thereof. In another embodiment, the salt may be, inter alia, a sodium salt.

In another embodiment the compound is characterized by the following structure:

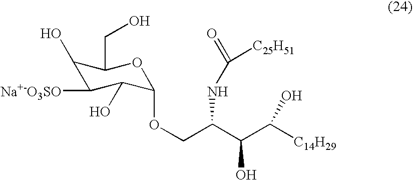

This invention provides, in one embodiment, a method for treating, delaying onset of, reducing incidence of, suppressing or reducing the severity of neoplasia in a subject, comprising the steps of:

a. culturing immature dendritic cells with a neoplastic cell;

b. contacting the culture in (a) with a compound characterized by the structure of the formula 11:

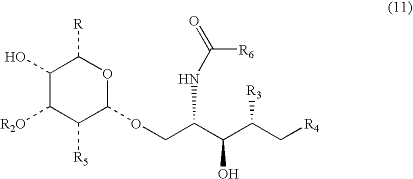

wherein,
   R═COOR₁ or CH₂OR₁;
   R₁═H or an alkyl group;
   R₂═H or SO₃⁻;
   R₃═H or OH;
   R₄═H, unsaturated or saturated, alkyl group;
   R₅═OH, acetamido or a halogen atom; and
   R₆═X-A
   A=
   dialkyl phenyl;

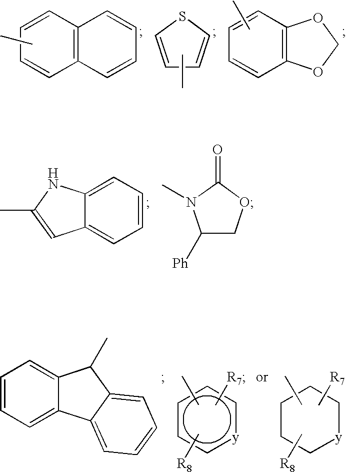

X=alkyl, alkenyl, alkoxy, thioalkoxy, substituted furan, or unsubstituted furan;
Y═N or C
R7=halogen, H, phenyl, alkyl, alkoxy, nitro or CF3; and
R8=methyl or H;
   or a pharmaceutically acceptable salt thereof; and

d. administering the culture in (b) to said subject.

In one embodiment, the compound is represented by the structure of formula 12:

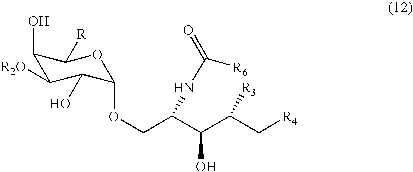

or a pharmaceutically acceptable salt thereof.

In another embodiment, the compound is represented by the structure of formula 3:

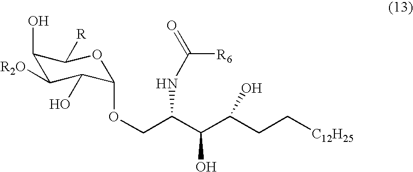

or a pharmaceutically acceptable salt thereof.

In another embodiment, the compound is represented by the structure of formula 4:

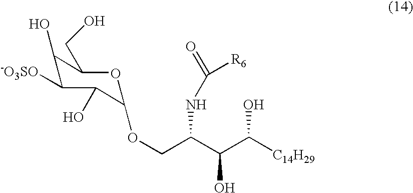

or a pharmaceutically acceptable salt thereof.

In another embodiment, the salt may be, inter alia, a sodium salt.

In another embodiment, the compound is represented by the structure of formula 15:

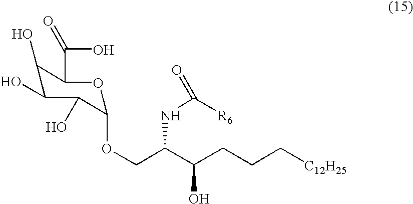

In another embodiment, the compound is represented by the structure of formula 16:

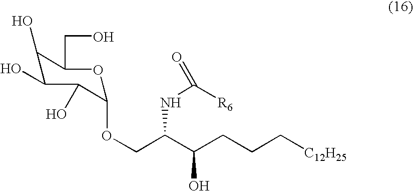

In one embodiment, the alkyl chain of R₄ has 1 carbon atom, in another embodiment, the alkyl chain of R₄ has between 1-5, or in another embodiment, 2-6, or in another embodiment, 3-7, or in another embodiment, 4-8, or in another embodiment 5-9 carbon atoms. In one embodiment, the alkyl chain of R₄ has 10-25 carbon atom, in another embodiment, the alkyl chain of R₄ has between 10-15 carbon atoms.

In another embodiment, the alkyl chain of R₄′ has 1 carbon atom, in another embodiment, the alkyl chain of R₄′ has between 1-10, or in another embodiment, 10-15, or in another embodiment, 5-13, or in another embodiment, 8-15, or in another embodiment 10-25 carbon atoms or, in another embodiment, between 20-30 carbon atoms.

In another embodiment, n is an integer ranging from 1-5, or, in another embodiment, between 5-10, or in another embodiment, 10-15, or in another embodiment, 10-20, or in another embodiment, 1-15, or in another embodiment 15-25 carbon atoms or, in another embodiment, between 10-30.

The compounds for use in the methods and compositions of this invention may be prepared by any process known in the art. For example, and in one embodiment, the process for the preparation of a compound represented by the structure of formula (4)

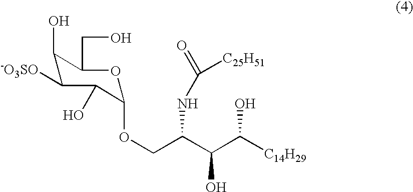

or a pharmaceutically salt thereof, the process including, inter alia, the step of:
removing the benzylidene protecting group and hydrogenating of the compound represented by the structure of formula (4a),

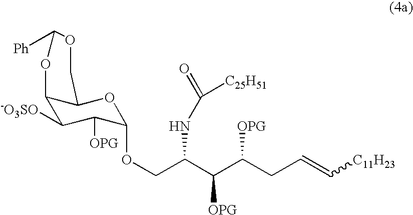

or a salt thereof, wherein PG is a hydroxy protecting group. In another embodiment, the hydroxy protecting group may be benzyl.

In one embodiment of the invention, the compound of formula (4a) may be obtained by a process including, inter alia, the step of:
conducting a selective sulfation of the 3″ OH of the galactose moiety of the compound represented by the structure of formula (4b):

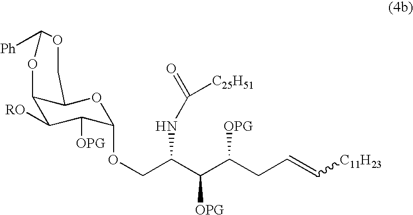

wherein PG is a hydroxy protecting group and R is H. In another embodiment, the hydroxy protecting group may be benzyl.

In one embodiment of the invention, the compound of formula (4b) wherein R is H, may be obtained by a process including, inter alia, the step of removing the levulinyl protecting group of a compound of formula (4b) wherein R is levulinyl, thereby obtaining a compound of formula (4b) wherein R is H.

In one embodiment of the invention, the compound of formula (4b) wherein R is levulinyl may be obtained by a process including, inter alia, the step of:
reacting a compound represented by the structure of formula (4c):

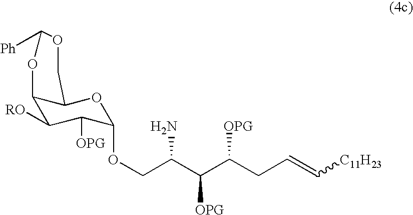

wherein R is H or levulinyl with hexacosanoic acid, thereby obtaining the compound of formula (4b) wherein R is levulinyl.

In one embodiment of the invention, the compound of formula (4c), wherein R is H or levulinyl, may be obtained by a process including, inter alia, the step of:
reducing the azide group of a compound represented by the structure of formula (4d):

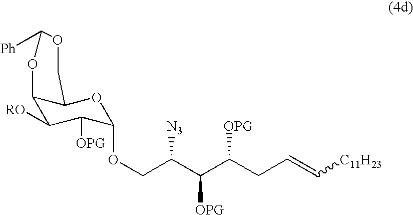

wherein R is levulinyl, thereby obtaining a compound of formula (4c) wherein R is H or levulinyl.

In one embodiment of the invention, the compound of formula (4d) wherein R is levulinyl, may be obtained by a process including, inter alia, the step of:
reacting a compound represented by the structure of formula (4e)

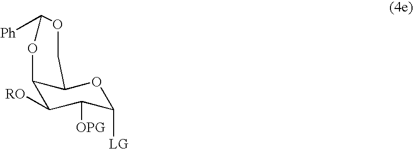

wherein PG is a hydroxy protecting group, LG is a leaving group and R is levulinyl, with a compound represented by the structure of formula (4f)

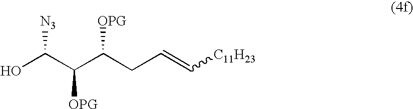

wherein PG is a hydroxy protecting group,
to form an alpha glycosidic bond, thereby obtaining the compound of formula (4d) wherein R is levulinyl. In another embodiment, the leaving group may be, inter alia,

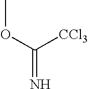

In one embodiment, the invention provides a process for the preparation of a compound represented by the structure of formula (10)

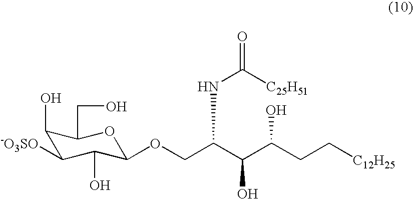

or a pharmaceutically salt thereof, including, inter alia, the step of:
conducting a selective sulfation of the 3″ OH of the galactose moiety of the compound represented by the structure of formula (10a):

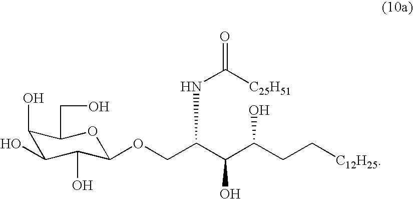

In another embodiment, the sulfation may be conducted in the presence of Bu₂SnO.

In one embodiment of the invention, the compound of formula (10a) may be obtained by the process including, inter alia, the step of:
removing the hydroxy protecting groups and hydrogenating the compound represented by the structure of formula (10b):

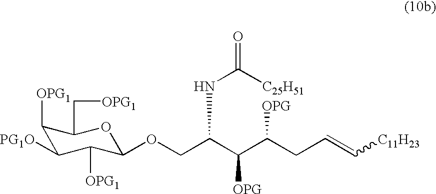

wherein PG and PG₁ are hydroxy protecting groups, thereby obtaining the compound of formula (10a). In another embodiment, the PG may be, inter alia, benzyl. In another embodiment, the PG1 may be, inter alia, benzoyl. In one embodiment of the invention, the compound of formula (10b) may be obtained by a process including, inter alia, the step of:
reacting a compound represented by the structure of formula (10c):

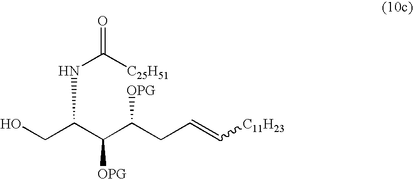

wherein PG is a hydroxy protecting group,
with a compound represented by the structure of formula (10d):

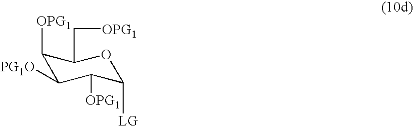

wherein PG₁ is a hydroxy protecting group and LG is a leaving group, thereby obtaining the compound of formula (10b). In another embodiment, the leaving group may be, inter alia,

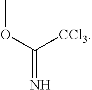

In one embodiment of the invention, the compound of formula (10c) may be obtained by a process comprising the steps of:
reducing the azide of a compound represented by the structure of formula (10e):

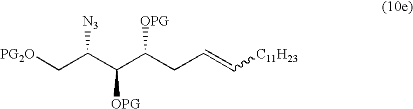

wherein PG and PG₂ are hydroxy protecting groups;
reacting the resulting amine with hexacosanoic acid; and
   removing the hydroxy protecting group PG₂, thereby obtaining the compound of formula (10c). In another embodiment, the PG₂ may be, inter alia, TIPS.

In one embodiment, the invention provides a process for the preparation of a compound represented by the structure of formula (II):

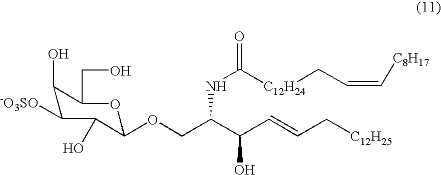

or a pharmaceutically salt thereof, including, inter alia, the step of:
conducting a selective sulfation of the 3″ OH of the galactose moiety of the compound represented by the structure of formula (11a):

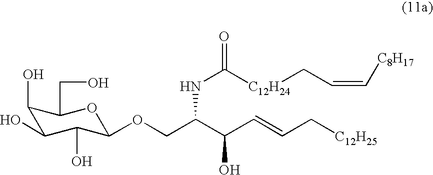

thereby obtaining the a compound represented by the structure of formula (10). In another embodiment, the sulfation may be conducted in the presence of Bu₂SnO.

In one embodiment of the invention, the compound of formula (11a) may be obtained by the process including, inter alia, the step of:
removing the hydroxy protecting groups of the compound represented by the structure of formula (11b):

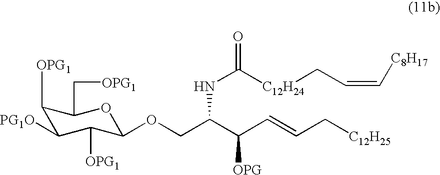

wherein PG and PG₁ are hydroxy protecting groups, thereby obtaining the compound of formula (11a). In another embodiment, PG may be, inter alia, benzoyl. In another embodiment, PG₁ may be, inter alia, benzoyl.

In one embodiment of the invention, the compound of formula (10b) may be obtained by a process including, inter alia, the step of:
deprotecting the amine of a compound represented by the structure of formula (11c):

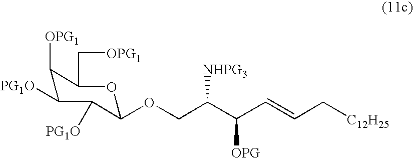

wherein PG and PG₁ are hydroxy protecting groups, and
PG₃ is an amino protecting group,
and reacting with nervonic acid, thereby obtaining the compound of formula (11b). In another embodiment, the amino protecting group may be, inter alia, tBoc.

In one embodiment, the term “a compound as herein described” refers to any compound which may be characterized by the structures of the formulas 1-16. In one embodiment, the term “a compound as herein described” refers to any compound which is described in the Examples section below. In another embodiment, the term “a compound as herein described” refers to any compound which may be produced by the process as described herein. In another embodiment, the term “a compound as herein described” specifically encompasses compound 24.

While theoretically, irradiated tumor cells represent an attractive target for treating and/or preventing neoplastic disease, in practice they have not proven to be sufficiently immunogenic.

As is exemplified herein, however, irradiated, MHC class I negative, mouse plasmacytoma cells were taken up by dendritic cells (DCs), which matured and then participated in an immune response, which was specific, with regard to the plasmacytoma cells.

In one embodiment, this invention provides a method for treating, delaying onset of, reducing incidence of, suppressing or reducing the severity of neoplasia in a subject, comprising the steps of culturing immature dendritic cells with a neoplastic cell, contacting the culture with a compound as described herein, and administering the culture to the subject.

In one embodiment, the dendritic cells are contacted with a compound as described herein in vitro, or in another embodiment, ex vivo.

In one embodiment, the term “contacting a cell” refers herein to both direct and indirect exposure of cell to the indicated item. In one embodiment, contact of any cell with a compound of this invention, a cytokine, growth factor, dendritic cell, or combination thereof, is direct or indirect. In one embodiment, contacting a cell may comprise direct injection of the cell through any means well known in the art, such as microinjection. It is also envisaged, in another embodiment, that supply to the cell is indirect, such as via provision in a culture medium that surrounds the cell, or administration to a subject, via any route well known in the art, and as described hereinbelow.

In one embodiment, the DCs are autologous, or in another embodiment, syngeneic, or in another embodiment, allogeneic, with respect to the subject to which the culture is administered. In one embodiment, the DCs are isolated from a subject having or predisposed to having neoplasia

In one embodiment, the term “dendritic cell” (DC) refers to antigen-presenting cells, which are capable of presenting antigen to T cells, in the context of CD1. In one embodiment, the dendritic cells utilized in the methods of this invention may be of any of several DC subsets, which differentiate from, in one embodiment, lymphoid or, in another embodiment, myeloid bone marrow progenitors.

In one embodiment, the methods of this invention may further employ the addition of cytokines or growth factors to the cultures as described herein, or in another embodiment, may comprise the compositions of this invention. In one embodiment, the cytokines and/or growth factors may serve to enhance, activate, or direct the developing immune response stimulated in the subject, by the administration of the compositions or cultures as herein described. In one embodiment, the cytokines and/or growth factors further promote maturation of the DCs, which, in another embodiment, present antigen to T cells in the subject. In another embodiment, NKT cells activated by the glycolipid, or other like molecules stimulate DC maturation. In one embodiment, CD4+ and/or CD8+ T cells then undergo expansion. In one embodiment, the cytokines may comprise IL-11, IL-6, TNF-α, PGE2, granulocyte-macrophage colony-stimulating-factor (GM-CSF), interleukin (IL)-3, or a combination thereof. In another embodiment, the cytokine and/or growth factor may serve to activate the immune system, or in another embodiment, promote a more robust response, or in another embodiment, promote the development of T cell memory.

In another embodiment, the DCs for use in the methods and/or compositions of this invention may be generated from proliferating progenitors isolated from bone marrow. In another embodiment, DCs may be isolated from CD34+ progenitors as described by Caux and Banchereau in Nature in 1992, or from monocytes, as described by Romani et al, J. Exp. Med. 180: 83-93 '94 and Bender et al, J. Immunol. Methods, 196: 121-135, '96 1996. In another embodiment, the DCs are isolated from blood, as described for example, in O'Doherty et al, J. Exp. Med. 178: 1067-1078 1993 and Immunology 82: 487-493 1994, all methods of which are incorporated fully herewith by reference.

In one embodiment, the DCs for use in the methods and/or compositions of this invention may express myeloid markers, such as, for example, CD11c or, in another embodiment, an IL-3 receptor-α (IL-3Rα) chain (CD123). In another embodiment, the DCs may produce type I interferons (IFNs). In one embodiment, the DCs for use in the methods and/or compositions of this invention express costimulatory molecules. In another embodiment, the DCs for use in the methods and/or compositions of this invention may express additional adhesion molecules, which may, in one embodiment, serve as additional costimulatory molecules, or in another embodiment, serve to target the DCs to particular sites in vivo, when delivered via the methods of this invention, as described further hereinbelow.

In one embodiment, the DCs may be obtained from in vivo sources, such as, for example, most solid tissues in the body, peripheral blood, lymph nodes, gut associated lymphoid tissue, spleen, thymus, skin, sites of immunologic lesions, e.g. synovial fluid, pancreas, cerebrospinal fluid, tumor samples, granulomatous tissue, or any other source where such cells may be obtained. In one embodiment, the dendritic cells are obtained from human sources, which may be, in another embodiment, from human fetal, neonatal, child, or adult sources. In another embodiment, the dendritic cells used in the methods and/or compositions of this invention may be obtained from animal sources, such as, for example, porcine or simian, or any other animal of interest. In another embodiment, dendritic cells used in the methods and/or compositions of this invention may be obtained from subjects that are normal, or in another embodiment, diseased, or in another embodiment, susceptible to a disease of interest, or in another embodiment, of a particular genetic profile, such as, for example, from an individual which is known to overexpress a particular gene, or in another embodiment, underexpress a particular gene, or in another embodiment, be from a population typically susceptible to a given neoplasia.

Dendritic cell separation may be accomplished, in another embodiment, via any of the separation methods as is known in the art. In one embodiment, positive and/or negative affinity based selections are conducted. In one embodiment, positive selection is based on CD86 expression, and negative selection is based on GR1 expression.

In another embodiment, the dendritic cells used in the methods and/or compositions of this invention may be generated in vitro by culturing monocytes in presence of GM-CSF and IL-4.

In one embodiment, the dendritic cells used in the methods and/or compositions of this invention may express CD83, an endocytic receptor to increase uptake of the autoantigen such as DEC-205/CD205 in one embodiment, or DC-LAMP (CD208) cell surface markers, or, in another embodiment, varying levels of the antigen presenting MHC class I and II products, or in another embodiment, accessory (adhesion and co-stimulatory) molecules including CD40, CD54, CD58 or CD86, or any combination thereof. In another embodiment, the dendritic cells may express varying levels of CD115, CD14, CD68 or CD32.

In one embodiment, the DCs are matured for effecting the methods of this invention. In one embodiment, maturation of the DC's occurs as a function of NKT cell activation by a compound of this invention, or a composition comprising the same, as exemplified herein.

In one embodiment, the term “mature dendritic cells” refers to a population of dendritic cells with diminished CD115, CD14, CD68 or CD32 expression, or in another embodiment, a population of cells with enhanced CD86 expression, or a combination thereof. In another embodiment, mature dendritic cells will exhibit increased expression of one or more of p55, CD83, CD40 or CD86 or a combination thereof. In another embodiment, the dendritic cells used in the methods and/or compositions of this invention will express the DEC-205 receptor on their surface. In another embodiment, CD40 ligation of, CpG oligodeoxyribonucleotide addition to, ligation of the IL-1, TNFα or TOLL like receptor ligand, bacterial lipoglycan or polysaccharide addition or activation of an intracellular pathway such as TRAF-6 or NF-κB may also be used to effect the methods of this invention and/or for preparing the compositions of this invention. It is to be understood that DC maturation via these and other means, known in the art and/or in combination with the use of cytokines and/or growth factors, may be utilized for effecting the methods and/or preparing the compositions of this invention and represent embodiments thereof.

In one embodiment, the maturation status of the dendritic may be confirmed, for example, by detecting either one or more of 1) an increase expression of one or more of p55, CD83, CD40 or CD86 antigens; 2) loss of CD115, CD14, CD32 or CD68 antigen; or 3) reversion to a macrophage phenotype characterized by increased adhesion and loss of veils following the removal of cytokines which promote maturation of PBMCs to the immature dendritic cells, by methods well known in the art, such as, for example, immunohistochemistry, FACS analysis, and others. In another embodiment, the maturation status of the DC is evidenced via NKT cell expansion, as described and exemplified herein.

In one embodiment, the dendritic cells used for the methods and/or in the compositions of this invention may express, or in another embodiment, may be engineered to express a costimulatory molecule.

In one embodiment, dendritic cells used for the methods of this invention are enriched for CD86high or CD80high expression.

In another embodiment, the dendritic cells used in the methods and/or in the compositions of this invention are selected for their capacity to expand NK T cells. In one embodiment, the DCs are isolated from progenitors or from blood for this purpose. In another embodiment, dendritic cells expressing high amounts of DEC-205/CD205 are used for this purpose.

In one embodiment, the compounds described herein are used at a concentration of between about 1 to about 1,000 ng/ml. In one embodiment, the compounds described herein are used at a concentration of between about 0.05 to about 200 μg/ml. In one embodiment, 10-50 ng/ml is used. The dendritic cells are, in one embodiment, cultured in the presence of the antigen for a sufficient time to allow for uptake of the neoplastic, hyperplastic or preneoplastic cells, and in another embodiment, presentation of antigens thereby derived.

In one embodiment, the compounds are delivered to dendritic cells in vivo in the steady state, which, in another embodiment, leads to expansion of disease specific T cells, for example, disease specific NKT cells. Antigen delivery in the steady state can be accomplished, in one embodiment, as described (Bonifaz, et al. (2002) Journal of Experimental Medicine 196: 1627-1638; Manavalan et al. (2003) Transpl Immunol. 11: 245-58).

In another embodiment, the dendritic cells for use in the methods and/or compositions of this invention are isolated from a subject having or predisposed to having neoplasia.

In one embodiment, the term “neoplasia” encompasses the process whereby one or more cells of an individual exhibiting abnormal growth characteristic. In one embodiment, such a process may comprise progression to the presence of a mass of proliferating cells in the individual. In another embodiment, neoplasia may refer to a very early stage in that only relatively few abnormal cell divisions have occurred. In one embodiment, the invention relates to an individual's predisposition to the development of a neoplasm. Without limiting the present invention in any way, increased levels of or expression profiles of biomarkers in an individual who has not undergone the onset of neoplasia, may be indicative of that individual's predisposition to developing neoplasia.

In one embodiment, the term “predisposed to having neoplasia” refers to an individual with a higher risk factor or likelihood for developing neoplasia, such as, for example, an individual with a family history of neoplasia, or in another embodiment, an individual expressing genes associated with particular cancers, such as, for example, the so-called breast cancer genes, as described, for example, in U.S. Patent Application Publication Number 2004001852.

Cancer is a disease that involves the uncontrolled growth (i.e., division) of cells. Some of the known mechanisms which contribute to the uncontrolled proliferation of cancer cells include growth factor independence, failure to detect genomic mutation, and inappropriate cell signaling. The ability of cancer cells to ignore normal growth controls may result in an increased rate of proliferation. Although the causes of cancer have not been firmly established, there are some factors known to contribute, or at least predispose a subject, to cancer. Such factors include particular genetic mutations (e.g., BRCA gene mutation for breast cancer, APC for colon cancer), exposure to suspected cancer-causing agents, or carcinogens (e.g., asbestos, UV radiation) and familial disposition for particular cancers such as breast cancer. In some embodiments, neoplastic, hyperplastic or preneoplastic cells for use in the methods and/or compositions of this invention may be obtained from individuals, or cell lines, exhibiting these phenomenon.

The cancer may be a malignant, in one embodiment or, in another embodiment, a non-malignant cancer. Cancers or tumors may include, but are not limited to biliary tract cancer; brain cancer; breast cancer; cervical cancer; choriocarcinoma; colon cancer; endometrial cancer; esophageal cancer; gastric cancer; intraepithelial neoplasms; lymphomas; liver cancer; lung cancer (e.g. small cell and non-small cell); melanoma; neuroblastomas; oral cancer; ovarian cancer; pancreas cancer; prostate cancer; rectal cancer; sarcomas; skin cancer; testicular cancer; thyroid cancer; and renal cancer, as well as other carcinomas and sarcomas. In one embodiment the cancer is hairy cell leukemia, chronic myelogenous leukemia, cutaneous T-cell leukemia, multiple myeloma, follicular lymphoma, malignant melanoma, squamous cell carcinoma, renal cell carcinoma, prostate carcinoma, bladder cell carcinoma, or colon carcinoma.

In another embodiment, the neoplasia which may be treated in accordance with the present invention and/or with compositions of this invention may include tumor cells occurring in the adrenal glands; bladder; bone; breast; cervix; endocrine glands (including thyroid glands, the pituitary gland, and the pancreas); colon; rectum; heart; hematopoietic tissue; kidney; liver; lung; muscle; nervous system; brain; eye; oral cavity; pharynx; larynx; ovaries; penis; prostate; skin (including melanoma); testicles; thymus; and uterus. Examples of such tumors include apudoma, choristoma, branchioma, malignant carcinoid syndrome, carcinoid heart disease, carcinoma (e.g., Walker, basal cell, basosquamous, Brown-Pearce, ductal, Ehrlich tumor, in situ, Krebs 2, Merkel cell, mucinous, non-small cell lung, oat cell, papillary, scirrhous, bronchiolar, bronchogenic, squamous cell, and transitional cell), plasmacytoma, melanoma, chondroblastoma, chondroma, chondrosarcoma, fibroma, fibrosarcoma, giant cell tumors, histiocytoma, lipoma, liposarcoma, mesothelioma, myxoma, myxosarcoma, osteoma, osteosarcoma, Ewing's sarcoma, synovioma, adenofibroma, adenolymphoma, carcinosarcoma, chordoma, mesenchymoma, mesonephroma, myosarcoma, ameloblastoma, cementoma, odontoma, teratoma, thymoma, trophoblastic tumor, adenocarcinoma, adenoma, cholangioma, cholesteatoma, cylindroma, cystadenocarcinoma, cystadenoma, granulosa cell tumor, gynandroblastoma, hepatoma, hidradenoma, islet cell tumor, Leydig cell tumor, papilloma, Sertoli cell tumor, theca cell tumor, leiomyoma, leiomyosarcoma, myoblastoma, myoma, myosarcoma, rhabdomyoma, rhabdomyosarcoma, ependymoma, ganglioneuroma, glioma, medulloblastoma, meningioma, neurilemmoma, neuroblastoma, neuroepithelioma, neurofibroma, neuroma, paraganglioma, paraganglioma nonchromaffin, angiokeratoma, angiolymphoid hyperplasia with eosinophilia, angioma sclerosing, angiomatosis, glomangioma, hemangioendothelioma, hemangioma, hemangiopericytoma, hemangiosarcoma, lymphangioma, lymphangiomyoma, lymphangiosarcoma, pinealoma, carcinosarcoma, chondrosarcoma, cystosarcoma phyllodes, fibrosarcoma, hemangiosarcoma, leiomyosarcoma, leukosarcoma, liposarcoma, lymphangiosarcoma, myosarcoma, myxosarcoma, ovarian carcinoma, rhabdomyosarcoma, sarcoma (e.g., Ewing's experimental, Kaposi's, and mast-cell), neoplasms and for other such cells.

A subject having a cancer, in one embodiment, is a subject that has detectable cancerous cells.

A subject at risk of developing a cancer is one who has a higher than normal probability of developing cancer. These subjects include, for instance, subjects having a genetic abnormality that has been demonstrated to be associated with a higher likelihood of developing a cancer, subjects having a familial disposition to cancer, subjects exposed to cancer causing agents (i.e., carcinogens) such as tobacco, asbestos, or other chemical toxins, and subjects previously treated for cancer and in apparent remission.

In one embodiment, the method employs contacting the culture of dendritic cells with a compound as herein described and administering the culture to the subject.

In one embodiment, the dendritic cell contacted with the compound as herein described is further contacted with a neoplastic, preneoplastic or hyperplastic cell. In one embodiment, a neoplastic cell is a tumor cell, and may be obtained from tumors, or tissue or body fluids containing tumor cells, surgically resected or retrieved in the course of a treatment for a cancer. In one embodiment, the tumor cell is non-viable, for example, is an ethanol-treated tumor cell and may be obtained from, in some embodiments, metastatic or primary cancers.

In one embodiment, the methods and/or compositions of this invention make use of two or more compounds as described herein.

In some embodiments, the neoplastic, preneoplastic or hyperplastic cells for use in the methods and/or compositions of this invention, will express a cancer-associated antigen, in one embodiment, preferentially, or in another embodiment, at a greater concentration, or in another embodiment, in a particular form.

A unique finding of this invention is that the cancer cell, when administered to a subject with compound 24, for example, did not require addition of any other antigen. In one embodiment, the methods of this invention may make use of a cancer cell, in the absence of exogenous antigen, wherein the cancer cell expresses a cancer antigen, or in another embodiment, wherein the cancer cell does not express a known cancer antigen.

In one embodiment, the cancer-associated antigen may be referred to as a tumor antigen, which in one embodiment, is a compound, such as a peptide or protein, associated with a tumor or cancer cell surface and which is capable of provoking an immune response when expressed on the surface of an antigen presenting cell in the context of an MHC molecule. Cancer antigens may represent an immunogenic portion of a tumor or cancer.

Cancer antigens are antigens that can potentially stimulate apparently tumor-specific immune responses. Some of these antigens are encoded, although not necessarily expressed, by normal cells. These antigens can be characterized as those that are normally silent (i.e., not expressed) in normal cells, those that are expressed only at certain stages of differentiation, and those that are temporally expressed such as embryonic and fetal antigens. Other cancer antigens are encoded by mutant cellular genes, such as oncogenes (e.g., activated ras oncogene), suppressor genes (e.g., mutant p53), fusion proteins resulting from internal deletions or chromosomal translocations. Still other cancer antigens can be encoded by viral genes such as, for example, those carried on RNA and DNA tumor viruses. Examples of tumor antigens include MAGE, MART-1/Melan-A, gp100, Dipeptidyl peptidase IV (DPPIV), adenosine deaminase-binding protein (ADAbp), cyclophilin b, Colorectal associated antigen (CRC)-C017-1A/GA733, Carcinoembryonic Antigen (CEA) and its immunogenic epitopes CAP-1 and CAP-2, etv6, aml1, Prostate Specific Antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gp100 Pmel117, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, Imp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, and c-erbB-2.

It is to be understood that any cell expressing a cancer antigen as herein described may be used in the methods and/or compositions of this invention, and represents an embodiment thereof.

Cancers or tumors and tumor-antigens associated with such tumors (but not exclusively), which may be treated, etc., by the methods and via the compositions of this invention may include acute lymphoblastic leukemia (etv6; aml1; cyclophilin b), B cell lymphoma (Ig-idiotype), glioma (E-cadherin; α-catenin; α-catenin; γ-catenin; p120ctn), bladder cancer (p21ras), biliary cancer (p21ras), breast cancer (MUC family; HER2/neu; c-erbB-2), cervical carcinoma (p53; p21ras), colon carcinoma (p21ras; HER2/neu; c-erbB-2; MUC family), colorectal cancer (Colorectal associated antigen (CRC)-C017-1A/GA733; APC), choriocarcinoma (CEA), epithelial cell-cancer (cyclophilin b), gastric cancer (HER2/neu; c-erbB-2; ga733 glycoprotein), hepatocellular cancer (α-fetoprotein), Hodgkins lymphoma (Imp-1; EBNA-1), lung cancer (CEA; MAGE-3; NY-ESO-1), lymphoid cell-derived leukemia (cyclophilin b), melanoma (p15 protein, gp75, oncofetal antigen, GM2 and GD2 gangliosides), myeloma (MUC family; p21ras), non-small cell lung carcinoma (HER2/neu; c-erbB-2), nasopharyngeal cancer (Imp-1; EBNA-1), ovarian cancer (MUC family; HER2/neu; c-erbB-2), prostate cancer (Prostate Specific Antigen (PSA) and its immunogenic epitopes PSA-1, PSA-2, and PSA-3; PSMA; HER2/neu; c-erbB-2), pancreatic cancer (p21ras; MUC family; HER2/neu; c-erbB-2; ga733 glycoprotein), renal (HER2/neu; c-erbB-2), squamous cell cancers of cervix and esophagus (viral products such as human papilloma virus proteins), testicular cancer (NY-ESO-1), T cell leukemia (HTLV-1 epitopes), and melanoma (Melan-A/MART-1; cdc27; MAGE-3; p21ras; gp100 Pmel117). Hyperplastic, preneoplastic or neoplastic cells expressing these tumor antigens may be used in the methods and/or compositions of this invention.

In one embodiment, the tumor cell should be so handled as to be incapable of growing and dividing after administration into the subject, such that they are dead or substantially in a state of no growth. It is to be understood that “dead cells” means a cell which do not have an intact cell or plasma membrane, or in another embodiment, one that will not divide in vivo.

In one embodiment, the neoplastic cells are suspended in a state of no growth as are known to skilled artisans and may be useful in the present invention. For example, cells may be irradiated prior to use such that they do not multiply. Tumor cells may be irradiated to receive a dose of 2500 cGy to prevent the cells from multiplying after administration. Alternatively, ethanol treatment, or use of any other fixative, as will be known to one skilled in the art, may result in dead cells.

The tumor cells for use in the methods and compositions of this invention can be prepared from virtually any type of tumor. The present invention contemplates the use of tumor cells from solid tumors, including carcinomas; and non-solid tumors, including hematologic malignancies. Examples of solid tumors from which tumor cells can be derived include sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma. Hematologic malignancies include leukemias, lymphomas, and multiple myelomas. The following are non-limiting preferred examples of tumor cells to be preserved according to the present invention: melanoma, including stage-4 melanoma; ovarian, including advanced ovarian; small cell lung cancer; leukemia, including and not limited to acute myelogenous leukemia; colon, including colon metastasized to liver; rectal, colorectal, breast, lung, kidney, and prostate cancer cells.

In one embodiment, the neoplastic, preneoplastic or hyperplastic cells are autologous, derived from the patient for whom treatment is intended. Vaccines comprising tumor cells prepared using the method of the invention can used for treatment of both solid and non-solid tumors, as exemplified above.

In another embodiment, the tumor cells are preferably of the same type as, most preferably syngeneic (e.g., autologous or tissue-type matched) to, the cancer which is to be treated. For purposes of the present invention, syngeneic refers to tumor cells that are closely enough related genetically that the immune system of the intended recipient will recognize the cells as “self”, e.g., the cells express the same or almost the same complement of HLA molecules. Another term for this is “tissue-type matched”. For example, genetic identity may be determined with respect to antigens or immunological reactions, and any other methods known in the art. Preferably the cells originate from the type of cancer which is to be treated, and more preferably, from the same patient who is to be treated. The tumor cells can be, although not limited to, autologous cells dissociated from biopsy or surgical resection specimens, or from tissue culture of such cells. In another embodiment, allogeneic cells and stem cells are also within the scope of the present invention.

Tumor cells for use in the present invention may be prepared, in some embodiments, as follows: Tumors are processed as described by Berd et al. (Cancer Res. 1986; 46:2572; see also U.S. Pat. No. 5,290,551; U.S. patent application Ser. No. 08/203,004, U.S. patent application Ser. No. 08/475,016, and U.S. patent application Ser. No. 08/899,905). The cells are extracted by dissociation, such as by enzymatic dissociation with collagenase, or, alternatively, DNase, or by mechanical dissociation such as with a blender, teasing with tweezers, mortar and pestle, cutting into small pieces using a scalpel blade, and the like. Mechanically dissociated cells can be further treated with enzymes as will be understood by the skilled artisan, to prepare a single cell suspension.

Tumor cells may be prepared, in another embodiment, by a method according to Hanna et al., U.S. Pat. No. 5,484,596. Briefly, tumor tissue is obtained from patients suffering from the particular solid cancer from which the vaccine is to be prepared. The tumor tissue is surgically removed from the patient, separated from any non-tumor tissue, and cut into small pieces, e.g., fragments 2-3 mm in diameter. The tumor fragments are then digested to free individual tumor cells by incubation in an enzyme solution.

In another embodiment, tumor cells can be prepared according to the following procedure (see Hanna et al., U.S. Pat. No. 5,484,596). The tissue dissociation procedure of Peters et al. (Cancer Research 1979; 39:1353-1360) can be employed using sterile techniques throughout under a laminar flow hood. Tumor tissue can be rinsed three times in the centrifuge tube with HBSS and gentamicin and transferred to a petri dish on ice. Scalpel dissection removal of extraneous tissue can be followed by tumor mincing into pieces approximately 2 to 3 mm in diameter. Tissue fragments are placed in a 75 ml flask with 20-40 ml of 0.14% (200 units/mil) Collagenase Type 1 (Sigma C-0130) and 0.1% (500 Kunitz units/ml) deoxylibonuclease type 1 (Sigma D-0876) (DNAase 1, Sigma D-0876) prewarmed to 37° C. Flasks are placed in a 37° C. water bath with submersible magnetic stirrers at a speed which cause tumbling, but not foaming. After a 30-minute incubation, free cells are decanted through three layers of sterile medium-wet nylon mesh (166t: Martin Supply Co., Baltimore, Md.) into a 50 ml centrifuge tube. The cells are centrifuged at 1200 rpm (250×g) in a refrigerated centrifuge for 10 minutes. The supernatant is poured off and the cells are resuspended in 5-10 ml of DNAase (0.1% in HBSS) and held at 37° C. for 5-10 minutes. The tube is filled with HBSS, washed by centrifugation, resuspended to 15 ml in HBSS and held on ice. The procedure is repeated until sufficient cells are obtained, usually three times for tumor cells. Cells from the different digests are then pooled, counted, and in one embodiment, incubated with the dendritic cells.

Tumor cells can be frozen, in another embodiment, if stored for extended periods of time. The cells may be frozen or cryopreserved according to any method known in the art, either before or after any modification to the cells (e.g., haptenization, lysis, etc.) has been made. For example, the dissociated cells may be stored frozen in a freezing medium (e.g., prepared from a sterile-filtered solution of 50 ml Human Serum Albumin [American Red Cross] added to 450 ml of RPMI 1640 (Mediatech) supplemented with L-glutamine and brought to an appropriate pH with NaOH), such as in a controlled rate freezer or in liquid nitrogen until needed. The cells are ready for use upon thawing. In some embodiments, the cells are thawed shortly before use, or stored for no more than a couple of days before use. In other embodiments, the cells may be washed once or twice, and then suspended in HBSS without phenol red.

In some embodiments, the concentration of dissociated tumor cells can be adjusted to about 5-10×10⁷/ml, or to about 10×10⁷ cells per ml, in HBSS and/or a freezing medium. The freezing medium can be a plain cell growth medium such as HBSS, or a medium or buffer complemented with HSA, sucrose, dextran, or mixtures thereof.

In some embodiments the concentration of neoplastic, preneoplastic or hyperplastic cells may be from about 10×10⁴ to 1×10⁸, or in another embodiment 1×10⁶ to about 25×10⁶, or in another embodiment, from about 2.5×10⁶ to about 7.5×10⁶, tumor cells suspended in a pharmaceutically acceptable carrier or diluent, such as, but not limited to, Hank's solution (HBSS), saline, phosphate-buffered saline, and water.

In another embodiment, the tumor cells are at a concentration of from about 5×10⁴ to about 5×10⁶ cells, for example; 5×10⁴, 5×10⁵, or 5×10⁶ tumor cells.

In some embodiments, the neoplastic, preneoplastic or hyperplastic cells or cell lines for use in the methods and/or compositions of this invention may be inactivated via any physical, chemical, or biological means of inactivation, including but not limited to irradiation (preferably with at least about 5,000 cGy, more preferably at least about 10,000 cGy, even more preferably at least about 20,000 cGy); or treatment with mitomycin-C (preferably at least 10 μg/mL; more preferably at least about 50 μg/mL).

In some embodiments, the neoplastic, preneoplastic or hyperplastic cells or cell lines for use in the methods and/or compositions of this invention may be inactivated via fixation with such agents as glutaraldehyde, paraformaldehyde, or formalin. They may also be in an ionic or non-ionic detergent, such as deoxycholate or octyl glucoside, or treated, for example, using Vaccinia Virus or Newcastle Disease Virus. If desired, solubilized cell suspensions may be clarified or subject to any of a number of standard biochemical separation procedures to enrich or isolate particular tumor-associated antigens or plurality of antigens. Preferably, tumor antigen associated with the outer membrane of tumor cells, or a plurality of tumor associated antigens is enriched. The degree of enrichment may be, in some embodiments, 10-fold or in other embodiments, 100-fold over that of a whole-cell lysate. Isolated antigens, recombinant antigens, or mixtures thereof may also be used, in some embodiments, in conjunction with the cells.

While earlier attempts to increase the immunogenicity of whole tumor cells and stimulate T cell-mediated tumor immunity using bacterial components (e.g. BCG or C. parvum) achieved only limited success, they implied a need for coordination between innate immunity and adaptive immunity against the tumors. These approaches, however, failed to provide for tumor cell access to maturing DCs to allow cross presentation of antigens by these potent and specialized antigen presenting cells.

Moreovoer, in the present invention, a single dose of irradiated nonmodified tumor cells, when directed to maturing DCs in vivo, exemplified here by i.v. administration, led to long-lived protective and combined CD4+ and CD8+ T cell immunity. The compound 24 maturation stimulus was shown to be superior relative to other DC stimuli such as ligation of CD40, TLR4, and TLR3, as exemplified herein, and the glycolipid was presented on CD1d molecules, which, in turn activate NKT lymphocytes, maturing the DCs, which participate in the antitumor response.

Thus, in one embodiment of the invention, the methods provide for the maturing of DC in response to innate NKT lymphocytes, as a result of the exposure to a compound as herein described. Tumor cells, when injected i.v., as exemplified herein, are captured by splenic DCs. Long lasting protection and therapeutic efficacy were induced with a single dose of tumor cells coadministered with the NKT mobilizing glycolipid, a compound as herein described, thus, in another embodiment, other glycolipids, in addition to compounds as herein described may be used to effect the methods of this invention. In one embodiment, the glycolipid will have structural, or in another embodiment, functional homology thereof, in that it will promote internalization of the tumor cell within the DC, cytokine elaboration, NKT cell stimulation and/or a combination thereof, to facilitate anti-neoplastic responses.

The resistance from one immunization lasted more than 2 months, as exemplified herein, and depended upon CD4+ and CD8+ T cells. Maturing DCs stimulated differentiation of P1A tumor antigen-specific, T cells and uniquely transferred resistance to naïve mice. Therefore, access of dying tumor cells to DCs maturing in response to innate NKT cells efficiently induces long lived adaptive resistance, and such access represents the methods of this invention, in embodiments thereof.

In one embodiment, the method provides for the expansion of NK T cells, and as such, in one embodiment, the method may further comprise the administration of NKT cells for promoting anti-neoplastic activity. In one embodiment, the NKT cells will be autologous, syngeneic or allogeneic, with respect to the dendritic cells, and in another embodiment, either the dendritic cells and/or the NKT cells will be autologous, syngeneic or allogeneic with respect to the subject.

In one embodiment, any one of the compounds of the invention may be a ligand for an NKT (natural killer T) cell. In another embodiment, the ligand may be in a complex with a CD1 molecule. In another embodiment, the CD1 molecule is a CD1d molecule. In another embodiment, the ligand stimulates NKT cells, which express a CD161+ NK marker as well as an invariant T cell antigen receptor (TCR) on the surface thereof.

In another embodiment, the invention provides a composition or vaccine including, inter alia, any one of the compounds of the invention. In another embodiment, the composition or vaccine may include, inter alia, at least one cell population. In another embodiment, the cell population may include, inter alia, NKT cells, antigen presenting cells, or a combination thereof.

In another embodiment, the invention provides a method for stimulating NKT cells, the method including, inter alia, contacting an NKT cell with any one of the compounds of the invention. In one embodiment, the NKT cells participate in the immune response to neoplastic or preneoplastic cells.

In one embodiment, a neoplastic or preneoplastic cell derived antigen is presented in the context of a CD1 molecule, which in one embodiment is CD1d. Activated NK T cells may display an NK-like perforin-dependent cytotoxicity against various cells, including tumor cells or cell lines and inhibit tumor metastasis, among other applications, as is described further hereinbelow, and representing embodiments of the methods of this invention.

The T cells used in, or stimulated by the methods of this invention may express CD161 and V 24i TCR on their cell surface. In one embodiment, the T cells may be classified as CD161high expressors, or in another embodiment, the T cells may be classified as CD161low expressors, or in another embodiment, a combination thereof.

In one embodiment, the T cell subpopulation, are “invariant NK T cells,” which may represent a major fraction of the mature T cells in thymus, the major T cell subpopulation in murine liver, and/or up to 5% of splenic T cells.

In another embodiment, the T cell subpopulation may be “non-invariant NK T cells”, which may comprise human and mouse bone marrow and human liver T cell populations that are, for example, CD1d-reactive noninvariant T cells which express diverse TCRs, and which can also produce a large amount of IL-4 and IFN-γ. In another embodiment, the NKT cells will bind CD1d-glycolipid multimers, as described and exemplified herein.

In one embodiment, NKT cells are contacted with DCs in vitro, and undergo expansion in culture, and are then administered to a subject, according to the methods of this invention.

In another embodiment, the invention provides a method for stimulating, inhibiting, suppressing or modulating an immune response in a subject, the method includes, inter alia, the step of contacting an NKT cell in the subject with any one of the compounds of the invention.

In one embodiment, the NK T or dendritic cells may be obtained from in vivo sources, such as, for example, peripheral blood, leukopheresis blood product, apheresis blood product, peripheral lymph nodes, gut associated lymphoid tissue, spleen, thymus, cord blood, mesenteric lymph nodes, liver, sites of immunologic lesions, e.g. synovial fluid, pancreas, cerebrospinal fluid, tumor samples, granulomatous tissue, or any other source where such cells may be obtained. In one embodiment, the cells are obtained from human sources, which may be, in another embodiment, from human fetal, neonatal, child, or adult sources. In another embodiment, the cells may be obtained from animal sources, such as, for example, porcine or simian, or any other animal of interest. In another embodiment, the cells may be obtained from subjects that are normal, or in another embodiment, diseased, or in another embodiment, susceptible to a disease, which, according to this invention is a neoplastic disease.

In one embodiment, sustained expansion of T cells, including, inter-alia, NKT cells, within a subject, may be accomplished via the methods of this invention, following contact with compound-pulsed dendritic cells.

Dendritic cells stimulated NK T cell cytokine production, when presenting a compound of this invention, in the context of CD1. In another embodiment of this invention, the stimulated NK T cells may induce maturation of the dendritic cells, which may be mediated via TCR and CD1d/glycolipid interactions, and engagement of the CD40/CD40L interaction. This in turn, in another embodiment, may promote IL-12 secretion by the dendritic cells, and/or upregulation of, inter-alia, MHC molecules, DEC-205, or costimulatory molecules such as the B7 family. Dendritic cell maturation as a result of this interaction, may, in another embodiment, lead to enhanced adaptive immune responses, which in another embodiment, includes adjuvant activity of the compounds of this invention.

Methods for priming dendritic cells with antigen are well known to one skilled in the art, and may be effected, as described for example Hsu et al., Nature Med. 2:52-58 (1996); or Steinman et al. International application PCT/US93/03141.

In one embodiment, a compound of this invention is added to a culture of dendritic cells prior to contact of the dendritic cells with NK T cells. In one embodiment, a compound of this invention is used at a concentration of between about 0.1 to about 200 μg/ml. In one embodiment, 10-50 μg/ml is used. The dendritic cells are, in one embodiment, cultured in the presence of the antigen for a sufficient time to allow for uptake and presentation, prior to, or in another embodiment, concurrent with culture with NK T cells.

In another embodiment, the compound is administered to the subject, and, in another embodiment, is targeted to the dendritic cell, wherein uptake occurs in vivo, for methods as described hereinbelow. Antigenic uptake and processing, in one embodiment, can occur within 24 hours, or in another embodiment, longer periods of time may be necessary, such as, for example, up to and including 4 days or, in another embodiment, shorter periods of time may be necessary, such as, for example, about 1-2 hour periods.

In one embodiment, NK T cells may be cultured with dendritic cells with a dendritic cell to T cell ratio of 10:1 to 1:1 to 1:10, which ratio, in some embodiments is dependent upon the purity of the NKT cell population used. In one embodiment, about 20,000-100,000 cells/well (96-well flat bottom plate) of a NKT cell line, or 5 million per ml T cells, or in another embodiment, 200,000-400,000 cells/well of enriched NKT are administered to a subject, for some of the methods of this invention.

In one embodiment, about 5 million T cells are administered to a subject, for some of the methods of this invention.

In another embodiment, the NK T cells expanded by the dendritic cells in the methods of this invention are autologous, syngeneic or allogeneic, with respect to the dendritic cells.

In one embodiment, the cells, as described herein, may be isolated from tissue, and, in another embodiment, an appropriate solution may be used for dispersion or suspension, toward this end. In another embodiment, the cells may be cultured in solution.

Such a solution may be, in another embodiment, a balanced salt solution, such as normal saline, PBS, or Hank's balanced salt solution, or others, each of which represents another embodiment of this invention. The solution may be supplemented, in other embodiment, with fetal calf serum, bovine serum albumin (BSA), normal goat serum, or other naturally occurring factors, and, in another embodiment, may be supplied in conjunction with an acceptable buffer. The buffer may be, in other embodiments, HEPES, phosphate buffers, lactate buffers, or the like, as will be known to one skilled in the art.

In another embodiment, the solution in which the cells may be placed is in medium is which is serum-free, which may be, in another embodiment, commercially available, such as, for example, animal protein-free base media such as X-VIVO 10™ or X-VIVO 15™ (BioWhittaker, Walkersville, Md.), Hematopoietic Stem Cell-SFM media (GibcoBRL, Grand Island, N.Y.) or any formulation which promotes or sustains cell viability. Serum-free media used, may, in another embodiment, be as those described in the following patent documents: WO 95/00632; U.S. Pat. No. 5,405,772; PCT US94/09622. The serum-free base medium may, in another embodiment, contain clinical grade bovine serum albumin, which may be, in another embodiment, at a concentration of about 0.5-5%, or, in another embodiment, about 1.0% (w/v). Clinical grade albumin derived from human serum, such as Buminate® (Baxter Hyland, Glendale, Calif.), may be used, in another embodiment.

In another embodiment, the cells may be separated via affinity-based separation methods. Techniques for affinity separation may include, in other embodiments, magnetic separation, using antibody-coated magnetic beads, affinity chromatography, cytotoxic agents joined to a monoclonal antibody or use in conjunction with a monoclonal antibody, for example, complement and cytotoxins, and “panning” with an antibody attached to a solid matrix, such as a plate, or any other convenient technique. In other embodiment, separation techniques may also include the use of fluorescence activated cell sorters, which can have varying degrees of sophistication, such as multiple color channels, low angle and obtuse light scattering detecting channels, impedance channels, etc. It is to be understood that any technique, which enables separation of the cells of or for use in this invention may be employed, and is to be considered as part of this invention.

In another embodiment, the affinity reagents employed in the separation methods may be specific receptors or ligands for the cell surface molecules indicated hereinabove.

In another embodiment, the antibodies utilized herein may be conjugated to a label, which may, in another embodiment, be used for separation. Labels may include, in other embodiments, magnetic beads, which allow for direct separation, biotin, which may be removed with avidin or streptavidin bound to, for example, a support, fluorochromes, which may be used with a fluorescence activated cell sorter, or the like, to allow for ease of separation, and others, as is well known in the art. Fluorochromes may include, in one embodiment, phycobiliproteins, such as, for example, phycoerythrin, allophycocyanins, fluorescein, Texas red, or combinations thereof.

In one embodiment, cell separations utilizing antibodies will entail the addition of an antibody to a suspension of cells, for a period of time sufficient to bind the available cell surface antigens. The incubation may be for a varied period of time, such as in one embodiment, for 5 minutes, or in another embodiment, 15 minutes, or in another embodiment, 30 minutes. Any length of time which results in specific labeling with the antibody, with minimal non-specific binding is to be considered envisioned for this aspect of the invention.

In another embodiment, the staining intensity of the cells can be monitored by flow cytometry, where lasers detect the quantitative levels of fluorochrome (which is proportional to the amount of cell surface antigen bound by the antibodies). Flow cytometry, or FACS, can also be used, in another embodiment, to separate cell populations based on the intensity of antibody staining, as well as other parameters such as cell size and light scatter.

The separated cells may be collected in any appropriate medium that maintains cell viability, and may, in another embodiment, comprise a cushion of serum at the bottom of the collection tube.

In another embodiment, the culture containing the cells for use in this invention may contain other cytokines or growth factors to which the cells are responsive. In one embodiment, the cytokines or growth factors promote survival, growth, function, or a combination thereof. In another embodiment, the culture containing the cells of or for use in this invention may contain polypeptides and non-polypeptide factors.

In another embodiment, the methods of this invention employ the use of immature dendritic cells, which are matured via the addition of a compound, as described and exemplified herein. In one embodiment, the immature dendritic cells are isolated from a subject with neoplasia, preneoplasia, hyperplasia, or a predisposition to neoplasia, or in another embodiment, from a subject having, or at enhanced risk of having neoplasia, such as, for example, a carcinoma or myeloma.

In one embodiment, the invention provides for the intravenous injection of compound pulsed DCs to a subject in need. As exemplified hereinbelow, such injection led to the uptake of tumor cells by DCs, as well as the capacity of the maturing DCs to present tumor antigen and induce protective immunity. Transfer of maturing DCs provided ˜60% mice with tumor protection while direct vaccination of dying tumor cells with compound 24 protected 80-100% of those mice.

The harnessing of DCs as exemplified herein, provided some advantages not previously observed with genetic modification of tumor cells to improve immunogenicity. First, maturing DCs expressed a plethora of cytokines, chemokines and accessory molecules as illustrated herein, rather than a single transduced costimulator, for example.

Second, because of the capacity of DCs to cross present cell associated antigens in vivo, the tumor cells become an effective source of antigen even if the tumor cells have dampened their own antigen presenting activities, as is often the case.

Third, by delivering tumor cells to the DCs, antigen presentation was enhanced beyond the presenting capacities of the tumor cells themselves, because DCs express such efficient processing pathways for MHC class I, class II, and CD1 as exemplified herein with the presentation of P1A, a classical tumor antigen, where the processing and presentation of the nonmutated P1A antigen from the tumor cells was found.

Fourth, the method provided for inducing combined innate (NKT) and adaptive (CD4, CD8) responses, and strong protective tumor immunity was achieved. The approach as provided in the methods of this invention takes full advantage of the positive feedback between NKT cells and DCs, which provide effector NKT cells as well as the extensive functional maturation of the antigen capturing DCs.

In another embodiment, this invention provides a method for treating, delaying onset of, reducing incidence of, suppressing or reducing the severity of neoplasia in a subject, comprising the step of administering to said subject a composition comprising a hyperplastic, preneoplastic or neoplastic cell and a compound as herein described.

In some embodiments of the invention, according to this aspect of the invention, the combined administration of the compound or compounds and the hyperplastic, preneoplastic or neoplastic cell results in DC uptake of the hyperplastic, preneoplastic or neoplastic cell, in vivo, which in turn promotes specific anti-tumor responses, as exemplified herein, and as described herein.

In other embodiments, the methods and/or compositions of this invention may comprise known cancer medicaments, such as those known to prime the immune system to attack the neoplastic, preneoplastic or hyperplastic cells. In other embodiments, methods and/or compositions of this invention may comprise known cancer medicaments such as angiogenesis inhibitors, which function by attacking the blood supply of solid tumors. Since the most malignant cancers are able to metastasize (i.e., exist the primary tumor site and seed a distal tissue, thereby forming a secondary tumor), medicaments that impede this metastasis are also useful in the treatment of cancer. Angiogenic mediators may include basic FGF, VEGF, angiopoietins, angiostatin, endostatin, TNF-α, TNP-470, thrombospondin-1, platelet factor 4, CAI, and certain members of the integrin family of proteins, and thus, in some embodiments, angiogenesis inhibitors may specifically targeted to prevent the activity or proper functioning of such molecules. In one embodiment, the inhibitor may comprise a metalloproteinase inhibitor, which inhibits the enzymes used by the cancer cells to exist the primary tumor site and extravasate into another tissue.

In other embodiments, the methods of this invention are for use in preventing neoplasia, or in another embodiment, preventing metastasis in a subject. Tumor metastasis involves the spread of tumor cells primarily via the vasculature to remote sites in the body. In one embodiment, the term “metastases” shall mean tumor cells located at sites discontinuous with the original tumor, usually through lymphatic and/or hematogenous spread of tumor cells. In one embodiment, the term metastasis refers to the invasion and migration of tumor cells away from the primary tumor site. A metastasis is, in some embodiments, a region of cancer cells, distinct from the primary tumor location resulting from the dissemination of cancer cells from the primary tumor to other parts of the body. At the time of diagnosis of the primary tumor mass, the subject may be monitored for the presence of metastases. Metastases are most often detected through the sole or combined use of magnetic resonance imaging (MRI) scans, computed tomography (CT) scans, blood and platelet counts, liver function studies, chest X-rays and bone scans in addition to the monitoring of specific symptoms.

The terms “prevent” and “preventing” as used herein with respect to metastasis refer to inhibiting completely or partially the metastasis of a cancer or tumor cell, as well as inhibiting any increase in the metastatic ability of a cancer or tumor cell.

The invasion and metastasis of cancer is a complex process which involves changes in cell adhesion properties which allow a transformed cell to invade and migrate through the extracellular matrix (ECM) and acquire anchorage-independent growth properties. Liotta, L. A., et al., Cell 64:327-336 (1991). Some of these changes occur at focal adhesions, which are cell/ECM contact points containing membrane-associated, cytoskeletal, and intracellular signaling molecules. Metastatic disease occurs when the disseminated foci of tumor cells seed a tissue which supports their growth and propagation, and this secondary spread of tumor cells is responsible for the morbidity and mortality associated with the majority of cancers.

In some embodiments, the methods of this invention and/or compositions of this invention specifically make use of cells at the initiation of, or during metastasis, as a means of treating, or in another embodiment, preventing, or in another embodiment, delaying the onset of, or in another embodiment, halting the progression of metastasis.

In some embodiments, the methods and/or compositions of this invention provide for a long-lived systemic immune response, and may therefore be effective not only against the primary tumor, but also against metastatic cells sharing tumor antigen with the primary tumor. In some embodiments, the methods and/or compositions of this invention may be useful in combating multiple types of tumors, which may be somewhat related in terms of, for example, the antigens expressed or downregulated in such tumors and represent embodiments of this invention.

In one embodiment, the methods and/or compositions of this invention stimulate an immune or immunological response, which in some embodiments, refers to the ability to initiate, boost, or maintain the capacity for the host's immune system to react to a target substance, such as a foreign molecule, an allogeneic cell, or a tumor cell, at a level higher than would otherwise occur. Stimulating a “primary” immune response refers herein to eliciting specific immune reactivity in a subject in which previous reactivity was not detected; for example, due to lack of exposure to the target antigen, refractoriness to the target, or immune suppression. Stimulating a “secondary” response refers to the reinitiation, boosting, or maintenance of reactivity in a subject in whom previous reactivity was detected; for example, due to natural immunity, spontaneous immunization, or treatment using one or several compositions or procedures. It is to be understood that the initiation of primary and/or secondary responses via the methods and/or compositions of this invention represent embodiments of the same.

In one embodiment, this invention provides a method of stimulating or enhancing an immune response in a subject, comprising contacting immune cells in the subject with a compound as described herein. In one embodiment, the method may further comprise contacting an immature DC with a compound as described herein.

In one embodiment of the invention, the immune response is biased toward Th1 or Th2. In another embodiment, the subject suffers from, or is at an elevated risk for an autoimmune disease. In another embodiment, the biasing of the immune response results in the suppression, inhibition or abrogation of the autoimmune disease. In another embodiment, the subject has an inappropriate or undesirable immune response. In another embodiment, the response is inflammatory. In another embodiment, the inappropriate or undesirable response exacerbates an infection, disease or symptom in the subject.

In one embodiment of the invention, the subject may be immunocompromised. In another embodiment, the subject is infected. In another embodiment, the subject is infected with HIV. In another embodiment, the subject is infected with mycobacteria. In another embodiment, the subject is infected with malaria. In another embodiment, the subject is infected with HIV, inycobacteria, or malaria.

In one embodiment of the invention, the subject is afflicted with cancer. In one embodiment of the invention, the subject is at an elevated risk for cancer. In one embodiment of the invention, the subject has precancerous precursors.

In another embodiment, the invention provides an adjuvant including, inter alia, any one of the compounds according to the invention.

In another embodiment, the invention provides a method of enhancing immunogenicity of a compound, composition, or vaccine in a subject, the method includes, inter alia, administering to the subject a compound, composition or vaccine further comprising an adjuvant of according to the invention, wherein the adjuvant enhances the immunogenicity of the compound, composition or vaccine.

In one embodiment, the invention provides a method of stimulating or enhancing cytokine production in a subject, the method includes, inter alia, administering to the subject any one of the compounds of the invention, whereby an NKT cell in the subject secretes a cytokine following contact with the compound, or in another embodiment, DC uptake of the compound. In another embodiment, the cytokine may be interferon-γ or Interleukin-4. In another embodiment, the method comprises stimulating additional or other T cell subsets.

In one embodiment, the T cells will be of one or more specificities, and may include, in another embodiment, those that recognize a mixture of antigens derived from an antigenic source. In one embodiment, a mixture of the compounds of this invention may be used to simulate a NK T cells and/or other T cells, such as T helper, T regulatory or cytotoxic T cells of varying specificity.

In one embodiment, the T cell population suppresses an autoimmune response. In one embodiment, the term “autoimmune response” refers to an immune response directed against an auto- or self-antigen. In one embodiment, the autoimmune response is rheumatoid arthritis, multiple sclerosis, diabetes mellitus, myasthenia gravis, pernicious anemia, Addison's disease, lupus erythematosus, Reiter's syndrome, atopic dermatitis or Graves disease. In one embodiment, the autoimmune disease caused in the subject is a result of self-reactive T cells, which recognize multiple self-antigens.

In another embodiment, the T cell population suppresses an inflammatory response. In one embodiment, the term “inflammatory response” refers to any response that is, in one embodiment, caused by inflammation or, in another embodiment, whose symptoms include inflammation. By way of example, an inflammatory response may be a result of septic shock, or, in another embodiment, a function of rheumatoid arthritis. The inflammatory response may be a part of an overall inflammatory disorder in a subject, and may comprise, in another embodiment, cardiovascular disease, rheumatoid arthritis, multiple sclerosis, Crohn's disease, inflammatory bowel disease, systemic lupus erythematosis, polymyositis, septic shock, graft versus host disease, host versus graft disease, asthma, rhinitis, psoriasis, cachexia associated with cancer, or eczema. In one embodiment, as described hereinabove, the inflammation in the subject may be a result of T cells, which recognize multiple antigens in the subject. In one embodiment, NK T cells of this invention may be specific for a single antigen where multiple antigens are recognized, yet the NK T cell population effectively suppresses inflammation in the subject. In one embodiment, suppression of inflammation is via modulating an immune response as a result of production of a particular cytokine profile. In one embodiment, NK T cells or T regulatory cells produce cytokines which serve to downmodulate the inflammatory response.

In another embodiment, the T cell populations of this invention suppresses an allergic response. In one embodiment, the term “allergic response” refers to an immune system attack against a generally harmless, innocuous antigen or allergen. Allergies may in one embodiment include, but are not limited to, hay fever, asthma, atopic eczema as well as allergies to poison oak and ivy, house dust mites, bee pollen, nuts, shellfish, penicillin or other medications, or any other compound or compounds which induce an allergic response. In one embodiment, multiple allergens elicit an allergic response, and the antigen recognized by the NK T cells of this invention may be any antigen thereof. In one embodiment, suppression of allergic responses is via modulating an immune response as a result of production of a particular cytokine profile. In one embodiment, the NK T cells produce cytokines which serve to downmodulate the allergic response.

In another embodiment, the T cells of the present invention are utilized, in circumstances wherein eliciting a “Th1” response is beneficial in a subject, wherein the subject has a disease where a so-called “Th2” type response has developed. Introduction of the T cells, in one embodiment, results in a shift toward a Th1 type response, in response to the cytokine profile produced from the T cells.

In one embodiment, the term “Th2 type response” refers to a pattern of cytokine expression, elicited by T Helper cells as part of the adaptive immune response, which support the development of a robust antibody response. Typically Th2 type responses are beneficial in helminth infections in a subject, for example. Typically Th2 type responses are recognized by the production of interleukin-4 or interleukin 10, for example.

In one embodiment, the term “Th1 type response” refers to a pattern of cytokine expression, elicited by T Helper cells as part of the adaptive immune response, which support the development of robust cell-mediated immunity. Typically Th1 type responses are beneficial in intracellular infections in a subject, for example. Typically Th1 type responses are recognized by the production of interleukin-2 or interferon γ, for example.

In another embodiment, the reverse occurs, where a Th1 type response has developed, when Th2 type responses provide a more beneficial outcome to a subject, where introduction of the NK T cells, vaccines or compositions of the present invention provides a shift to the more beneficial cytokine profile. One example would be in leprosy, where the NK T cells, vaccines or compositions of the present invention stimulates a Th1 cytokine shift, resulting in tuberculoid leprosy, as opposed to lepromatous leprosy, a much more severe form of the disease, associated with Th2 type responses.

In another embodiment, the T cells of this invention, and obtained via the methods of this invention, may be a part of a vaccine or composition. Such vaccines and/or compositions may be used in any applicable method of this invention, and represents an embodiment thereof.

For example, in one embodiment, the methods of this invention for stimulating, inhibiting, suppressing or modulating an immune response in a subject, which comprise contacting a T cell in a subject with a compound of the invention, may also comprise contacting the T cell with a compound in a composition, or in another embodiment, contacting the T cell with a vaccine comprising at least one compound of the invention.

It is to be understood that any use of the DC, T cells, vaccines or compositions of the present invention for methods of enhancing immunogenicity, such as, for example, for purposes of immunizing a subject to prevent disease, and/or ameliorate disease, and/or alter disease progression are to be considered as part of this invention.

Examples of infectious virus to which stimulation of a protective immune response is desirable, which may be accomplished via the methods of this invention, or utilizing the DC, T cells, vaccines or compositions of the present invention include: Retroviridae (e.g., human immunodeficiency viruses, such as HIV-1 (also referred to as HTLV-III, LAV or HTLV-III/LAV, or HIV-III; and other isolates, such as HIV-LP; Picornaviridae (e.g., polio viruses, hepatitis A virus; enteroviruses, human coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g. influenza viruses); Bungaviridae (e.g., Hantaan viruses, bunga viruses, phleboviruses and Nairo viruses); Arena viridae (hemorrhagic fever viruses); Reoviridae (erg., reoviruses, orbiviurses and rotaviruses); Birnaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes viruses′); Poxyiridae (variola viruses, vaccinia viruses, pox viruses); and Iridoviridae (e.g. African swine fever virus); and unclassified viruses (e.g., the etiological agents of Spongiform encephalopathies, the agent of delta hepatities (thought to be a defective satellite of hepatitis B virus), the agents of non-A, non-B hepatitis (class 1=internally transmitted; class 2=parenterally transmitted (i.e., Hepatitis C); Norwalk and related viruses, and astroviruses).

Examples of infectious bacteria to which stimulation of a protective immune response is desirable, which may be accomplished via the methods of this invention, or utilizing the DC, T cells, vaccines or compositions of the present invention include: Helicobacter pylori, Borellia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Chlamidia sp., Haemophilus influenzae, Bacillus antracis, corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Actinomyces israelli and Francisella tularensis.

Examples of infectious fungi to which stimulation of a protective immune response is desirable, which may be accomplished via the methods of this invention, or utilizing the DC, T cells, vaccines or compositions of the present invention include: Cryptococcus neoformans, Histoplasma capsulatum, Coccidioides immitis, Blastomyces dermatitidis, Chlamydia trachomatis, Candida albicans. Other infectious organisms (i.e., protists) include: Plasmodium sp., Leishmania sp., Schistosoma sp. and Toxoplasma sp.

In one embodiment, this invention provides a method for modulating an immune response, which is an inappropriate or undesirable response. In one embodiment, the immune response is marked by a cytokine profile which is deleterious to the host.

In one embodiment, the NK T cells of this invention may be administered to a recipient contemporaneously with treatment for a particular disease, such as, for example, contemporaneous with standard anti-0r therapy, to serve as adjunct treatment for a given cancer. In another embodiment, the NK T cells of this invention may be administered prior to the administration of the other treatment.

In another embodiment, this invention provides a method for modulating an immune response, which is directed to infection with a pathogen, and the immune response is not protective to the subject.

In one embodiment, the pathogen may mimic the subject, and initiate an autoimmune response.

In another embodiment, infection with the pathogen results in inflammation, which damages the host. In one embodiment, the response results in inflammatory bowel disease, or in another embodiment, gastritis, which may be a result, in another embodiment, of H. pylori infection.

In another embodiment, the immune response results in a cytokine profile, which is not beneficial to the host. In one embodiment, the cytokine profile exacerbates disease. In one embodiment, a Th2 response is initiated when a Th1 response is beneficial to the host, such as for example, in lepromatous leprosy. In another embodiment, a Th1 response is initiated, and persists in the subject, such as for example, responses to the egg antigen is schistosomiasis.

According to this aspect, and in one embodiment, administration of the NK T cells alters the immune response initiated in the subject, was not beneficial to the subject. In another embodiment, the method may further comprise the step of administering an agent to the subject, which if further associated with protection from the pathogen.

In one embodiment, the term “modulating” refers to initiation, augmentation, prolongation, inhibition, suppression or prevention of a particular immune response, as is desired in a particular situation. In one embodiment, modulating results in diminished cytokine expression, which provides for diminished immune responses, or their prevention. In another embodiment, modulation results in the production of specific cytokines which have a suppressive activity on immune responses, or, in another embodiment, inflammatory responses in particular. In another embodiment, modulating results in enhanced cytokine expression, which provides for enhanced immune responses, or their stimulation. In another embodiment, modulation results in the production of specific cytokines which have a stimulatory activity on immune responses, or, in another embodiment, responses to infection, or neoplasia, in particular.

In one embodiment, this invention provides a method for modulating an immune response in a subject, comprising the steps of contacting a dendritic cell population in vivo with compound of this invention, whereby the dendritic cell population contacts NK T cells in the subject, wherein NK T cell contact promotes cytokine production from the NK T cell population, thereby modulating an immune response in a subject.

In one embodiment, the term “modulating” refers to stimulating, enhancing or altering the immune response. In one embodiment, the term “enhancing an immune response” refers to any improvement in an immune response that has already been mounted by a mammal. In another embodiment, the term “stimulating an immune response” refers to the initiation of an immune response against an antigen of interest in a mammal in which an immune response against the antigen of interest has not already been initiated. It is to be understood that reference to modulation of the immune response may, in another embodiment, involve both the humoral and cell-mediated arms of the immune system, which is accompanied by the presence of Th2 and Th1 T helper cells, respectively, or in another embodiment, each arm individually. For further discussion of immune responses, see, e.g., Abbas et al. Cellular and Molecular Immunology, 3rd Ed., W. B. Saunders Co., Philadelphia, Pa. (1997).

Modulation of an immune response can be determined, in one embodiment, by measuring changes or enhancements in production of specific cytokines and/or chemokines for either or both arms of the immune system. In one embodiment, modulation of the immune response resulting in the stimulation or enhancement of the humoral immune response, may be reflected by an increase in IL-6, which can be determined by any number of means well known in the art, such as, for example, by ELISA or RIA. In another embodiment, modulation of the immune response resulting in the stimulation or enhancement of the cell-mediated immune response, may be reflected by an increase in IFN-γ or IL-12, or both, which may be similarly determined.

In one embodiment, stimulating, enhancing or altering the immune response is associated with a change in cytokine profile. In another embodiment stimulating, enhancing or altering the immune response is associated with a change in cytokine expression. Such changes may be readily measured by any number of means well known in the art, including as described herein, ELISA, RIA, Western Blot analysis, Northern blot analysis, PCR analysis, RNase protection assays, and others.

In another embodiment, this invention provides a method for producing an isolated, culture-expanded T cell population, comprising contacting T cells with dendritic cells and a compound of this invention, for a period of time resulting in antigen-specific T cell expansion and isolating the expanded T cells thus obtained, thereby producing an isolated, culture-expanded T cell population. In some embodiments, the method comprises contacting Vα14i, or Vα24i T cells with dendritic cells and a compound of this invention, and isolating the expanded T cells thus obtained, thereby producing an isolated, culture-expanded NK T cell population.

In one embodiment, the method for producing an isolated culture-expanded T cell population, further comprises the step of adding a cytokine or growth factor to the dendritic cell, T cell culture. In one embodiment, T cells secretion of interleukin-2, interferon-γ or interleukin-4 is detected, at which time the T cells may be used in the methods of this invention.

In another embodiment, this invention provides a method for delaying onset, reducing incidence or suppressing a disease in a subject, comprising the steps of contacting in a culture T cells with dendritic cells and a compound of this invention, for a period of time resulting in T cell expansion, cytokine production or a combination thereof, and administering the DC and/or T cells thus obtained to the subject, resulting in a delayed onset, reduced incidence or suppression of a disease in the subject.

It is to be understood that the modulation of any immune response, via the use of the DC, T cell populations, vaccines or compositions of this invention are to be considered as part of this invention, and an embodiment thereof.

In one embodiment, the methods and/or compositions of this invention are for the treatment of cancer. In one embodiment, the term “treatment” refers to intervention in an attempt to alter the natural course of the individual or cell being treated, and may be performed either for prophylaxis or during the course of clinical pathology. Desirable effects include preventing occurrence or recurrence of disease, alleviation of symptoms, diminishment of any direct or indirect pathological consequences of the disease, preventing metastasis, lowering the rate of disease progression, amelioration or palliation of the disease state, and remission or improved prognosis.

The “pathology” associated with a disease condition is anything that compromises the well-being, normal physiology, or quality of life of the affected individual. This may involve (but is not limited to) destructive invasion of affected tissues into previously unaffected areas, growth at the expense of normal tissue function, irregular or suppressed biological activity, aggravation or suppression of an inflammatory or immunological response, increased susceptibility to other pathogenic organisms or agents, and undesirable clinical symptoms such as pain, fever, nausea, fatigue, mood alterations, and such other features as may be determined by an attending physician.

An “effective amount” is an amount sufficient to effect a beneficial or desired clinical result, particularly the generation of an immune response, or noticeable improvement in clinical condition. An immunogenic amount is an amount sufficient in the subject group being treated (either diseased or not) to elicit an immunological response, which may comprise either a humoral response, a cellular response, or both. In terms of clinical response for subjects bearing a neoplastic disease, an effective amount is amount sufficient to palliate, ameliorate, stabilize, reverse or slow progression of the disease, or otherwise reduce pathological consequences of the disease. An effective amount may be given in single or divided doses. It is to be understood that the methods and/or compositions of this invention may provide an immunogenic or therapeutically effective amount, both of which are to be considered embodiments of this invention.

In some embodiments, the treatment can be ascertained via standard protocols for monitoring the tumor such as, for example, via the use of magnetic resonance imaging (MRI), radioscintigraphy with a suitable imaging agent, monitoring of circulating tumor marker antigens, the subject's clinical response, or a combination thereof. For example, and in one embodiment, an appropriate clinical marker is serum CA-125 for the monitoring of advanced ovarian cancer. Hempling et al. (1993) J. Surg. Oncol. 54:38-44.

The administration of the compositions and/or cells according to the methods of this invention may be conducted as appropriate, for example on a monthly, semimonthly, or in another embodiment, on a weekly basis, until the desired effect is achieved. Thereafter, and particularly when the immunological or clinical benefit appears to subside, additional booster or maintenance regimens may be undertaken, and designed as appropriate, as will be appreciated by one skilled in the art.

When multiple doses of a cellular vaccine are given to the same patient, some attention should be paid to the possibility that the allogeneic lymphocytes in the vaccine may generate an anti-allotype response. The use of a mixture of allogeneic cells from a plurality of donors, and the use of different allogeneic cell populations in each dose, are both strategies that can help minimize the occurrence of an anti-allotype response.

During the course of therapy, the subject is evaluated on a regular basis for side effects at the injection site, or general side effects such as a febrile response. Side effects are managed with appropriate supportive clinical care.

In another embodiment, this invention provides a composition comprising an immature dendritic cell, a hyperplastic, preneoplastic or neoplastic cell and a compound as herein described at an amount sufficient to stimulate dendritic cell phagocytosis of said hyperplastic, preneoplastic or neoplastic cell and maturation of said dendritic cell.

In another embodiment, this invention provides a composition comprising a hyperplastic, preneoplastic or neoplastic cell and a compound as herein described.

According to these aspects of the invention, and in one embodiment, the compositions of this invention and/or for use in the methods of this invention may be at a dose and schedule, which will vary depending on the age, health, sex, size and weight of the subject to which it will be administered. These parameters can be determined for each system by well-established procedures and analysis e.g., in phase I, II and III clinical trials, or other means, as will be appreciated by one skilled in the art.

For administration, the cells and compounds as herein described can be combined with a pharmaceutically acceptable carrier such as a suitable liquid vehicle or excipient and an optional auxiliary additive or additives. The liquid vehicles and excipients are conventional and are commercially available. Illustrative thereof are distilled water, physiological saline, aqueous solutions of dextrose and the like.

Suitable formulations for parenteral, topical, mucosal, for example, oral, intranasal, etc., or intraperitoneal administration, include aqueous solutions of active compounds in water-soluble or water-dispersible form. In addition, suspensions of the active compounds as appropriate oily injection suspensions may be administered. Suitable lipophilic solvents or vehicles include fatty oils for example, sesame oil, or synthetic fatty acid esters, for example, ethyl oleate or triglycerides. Aqueous injection suspensions may contain substances which increase the viscosity of the suspension, include for example, sodium carboxymethyl cellulose, sorbitol and/or dextran, optionally the suspension may also contain stabilizers. In other embodiments, the cells can be mixed with immune adjuvants well known in the art such as Freund's complete adjuvant, inorganic salts such as zinc chloride, calcium phosphate, aluminum hydroxide, aluminum phosphate, saponins, polymers, lipids or lipid fractions (Lipid A, monophosphoryl lipid A), modified oligonucleotides, etc.

General procedures for the preparation and administration of pharmaceutical compositions are outlined in Remington's Pharmaceutical Sciences 18th Edition (1990), E. W. Martin ed., Mack Publishing Co., PA, and represent embodiments of this invention.

In addition to administration with conventional carriers, the cells and other active ingredients may be administered by a variety of specialized delivery drug techniques which are known to those of skill in the art. The following examples are given for illustrative purposes only and are in no way intended to limit the invention.

It is to be understood that any disease, disorder or condition, whereby such disease, disorder or condition may be positively affected by the production of a given cytokine profile, or in another embodiment, is positively affected by the presence of NK T cells, and may be so positively affected via a method of this invention, is to be considered as part of this invention.

The following non-limiting examples may help to illustrate some embodiments of the invention.

### EXAMPLES

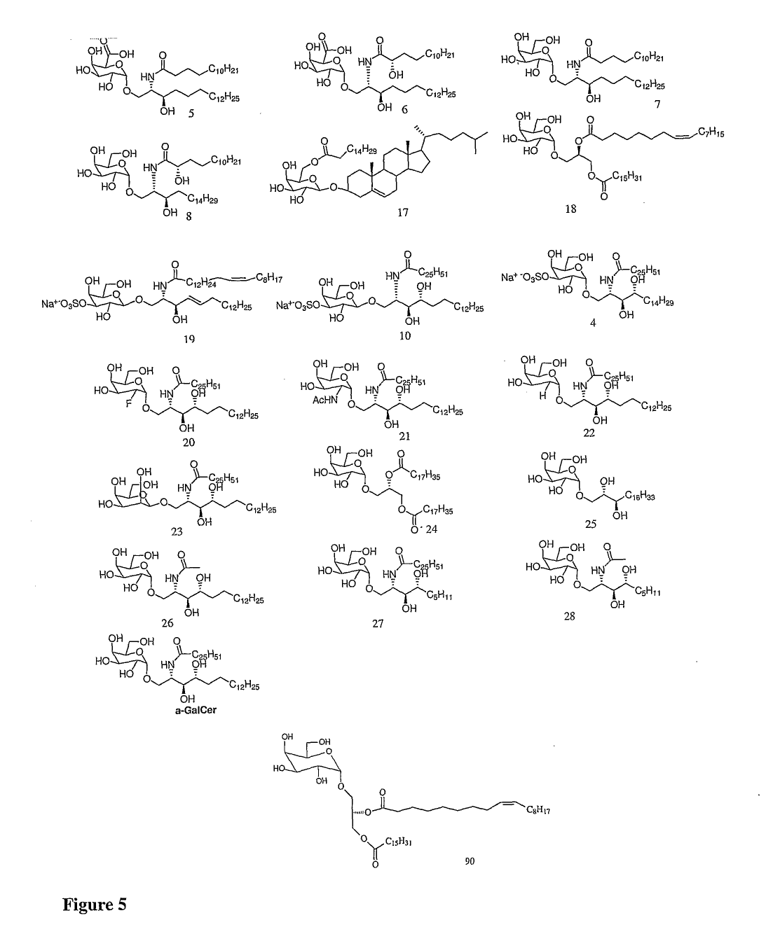

A number of glycolipids were synthesized (FIG. 5) and tested them for NKT cell activation. These included glycolipids of bacterial origin (compounds 5, 6, 17, and 18), α-GalCer analogues modified on the galactose moiety and acyl group, and variations of sulfatide, the only known promiscuous ligand for CD1. The bacterial glycolipids include those isolated from the outer membrane of Sphingomonas wittichii [Kawahara, K., Kubota, M., Sato, N., Tsuge, K. & Seto, Y. (2002) FEMS Microbiol Lett. 214, 289-294] and glycolipids from Borrelia burgdorferi, the Lyme disease spirochete. CD1d-deficient (CD1d^{-/-}) mice were shown to have impaired host resistance to infection by B. burgdorferi making its glycolipids attractive compounds for further study as possible natural CD1d antigens [Kumar, H., Belperron, A., Barthold, S. W. & Bockenstedt, L. K. (2000) J. Immunol. 165, 4797-4801]. The structures of its two major glycolipids were recently elucidated as cholesteryl 6-O-acyl-β-D-galactopyranoside 5 (B. burgdorferi glycolipid 1, BbGL-I) and 1,2-di-O-acyl-3-O-α-D-galactopyranosyl-sn-glycerol 6 (BbGL-II). The Sphingomonas glycolipids, two new α-linked glycosphingolipids 5 and 6, (GSL-1 and GSL-2 respectively) differ most significantly from α-GalCer in the carbohydrate moiety as they contain galactosyluronic acids as the polar head group [Ben-Menachem, G., Kubler-Kielb, J., Coxon, B., Yergey, A. & Schneerson, R. (2003) Proc. Natl. Acad. Sci. USA 100, 7913-7918]. However, they are more physiologically relevant as natural ligands for CD1d-mediated NKT-cell activation since they originate from bacteria. Biological experiments further show that galactouronic sphingolipids stimulate IL-2 secretion in 1.2 (Vα14 Vβ8.2 DN3A4) NKT cell hybridomas. An α-GalCer analogue 4,3-O-sulfo-galactosylceramide (3-O-sulfo-GalCer) also caused significant IL-2 secretion demonstrating that Vα14i NKT cell response is less sensitive to modification at the 3-OH position of galactose. By contrast, any modification made at the 2-OH position of galactose abolished all biological activity. Most other synthetic analogs, however, were active. In addition, reactivity of human Vα24i NKT cells to GSL-1 and GSL-2 and sulfatides were conserved.

### Example 1

### Synthesis of Analogs of Glycolipid α-Galactosyl Ceramide: 3-O-Sulfo-α-Galactosylceramide

### Preparation of Reagents

### Reagents

All chemicals were purchased as reagent grade and used without further purification. Dichloromethane (CH₂Cl₂, DCM) was distilled over calcium hydride and tetrahydrofuran (THF) over sodium/benzophenone. Anhydrous methanol (MeOH) and pyridine (Py) were purchased from a commercial source.

### General Assay Information:

Reactions were monitored with analytical thin-layer chromatography (TLC) on silica gel 60 F₂₅₄ glass plates and visualized under UV (254 nm) and/or by staining with acidic ceric ammonium molybdate. Flash column chromatography was performed on silica gel 60 Geduran (35-75 μm EM Science). ¹H NMR spectra were recorded on a 400- 500- or 600-Hz NMR spectrometer at 20° C. Chemical shift (in ppm) was determined relative to tetramethylsilane (δ0 ppm) in deuterated solvents. Coupling constant(s) in hertz (Hz) were measured from one-dimensional spectra. ¹³C Attached Proton Test (C-Apt) spectra were obtained with the NMR-400, 500 or 600 spectrometer (100, 125 or 150 Hz) and were calibrated with either CDCl₃ (δ77.23 ppm) or Py-d₅ (δ123.87 ppm).

### p-Methylphenyl 2-O-benzyl-4,6-O-benzylidene-3-O-levulinyl-1-thio-D-galactopyranoside (II)

3 grams of I (6.45 mmol) was dissolved in DCM. LevOH (0.9 ml, 1.35 eq), EDC (1.6 g, 1.3 eq) and DMAP (197 mg, 0.25 eq) were added. The reaction was allowed to proceed overnight while covered in foil. The reaction was then diluted with DCM, washed with water, saturated sodium bicarbonate solution, brine and dried over sodium sulfate. After removal of the solvent the mixture was purified by column chromatography (Hexanes:EtOAc:DCM 3:1:1) to give 2.83 g of II in 78% yield.

¹H(CDCl₃ 500 MHz) δ=7.61-7.03 (m, 14H), 5.48 (s, 1H), 4.98 (dd, J=3.7 Hz, J=9.6 Hz, 1H), 4.77 (d, J=11.0 Hz, 1H), 4.63 (d, J=9.5 Hz, 1H), 4.51 (d, J=11 Hz, 1H), 4.36-4.32 (m, 2H), 3.99-3.97 (m, 1H), 3.90-3.86 (m, 1H), 3.51 (s, 1H), 2.56-2.50 (m, 2H), 2.46-2.40 (m, 2H), 2.31 (s, 3H), 2.09, (s, 3H); ¹³C NMR (125 MHz, CDCl₃) δ=206.05, 172.09, 138.18, 137.76, 137.64, 133.11, 129.61, 128.98, 128.57, 128.22, 128.01, 127.68, 127.57, 126.45, 100.83, 86.53, 75.41, 75.05, 73.77, 73.71, 69.09, 37.70, 29.60, 27.99; HRMS (MALDI-FTMS) calcd. for C₃₂H₃₄O₇SNa [M+Na]⁺ 585.1923, found 585.1900.

### 2-O-benzyl-4,6-O-benzylidene-3-O-levulinyl-D-galactopyranoside (III)

II (600 mg, 1.07 mmol) was dissolved in 50 mL of acetone. The reaction mixture was cooled to 0° C., and NBS (228 mg, 1.28 mmol, 1.2 equiv) was added. The reaction mixture turned orange immediately. After 10 min the reaction was quenched by addition of solid NH₄Cl. The mixture was diluted with water and ethyl acetate, and the aqueous layer was extracted with ethyl acetate (3×). The combined organic layer was extracted with brine, dried over sodium sulfate, and evaporated. The residue was subjected to column chromatography (hexanes:ethyl EtOAc:DCM 1:1:1) to give 442 mg (91%) of 7.

¹H(CDCl₃ 500 MHz) δ=7.50-7.25 (m, 10H), 5.48 (d, J=4.8, 1H), 5.38 (s, 1H), 5.32 (dd, J=3.7 Hz, J=10.3 Hz, 1H), 4.94-4.90 (m, 1H), 4.73-4.62 (m, 3H), 4.36 (d, J=3.3 Hz, 1H), 4.05 (dd, J=3.3 Hz, 10.3 Hz, 1H), 4.00-3.98 (m, 2H), 3.93, (s, 1H), 3.52-3.51 (m, 1H), 2.71-2.53 (m, 4H), 2.08 (s, 3H); ¹³C NMR (125 MHz, CDCl₃) δ=206.43, 177.73, 172.35, 172.24, 138.41, 137.78, 137.63, 137.57, 128.89, 128.85, 128.38, 128.21, 128.03, 127.75, 127.67, 127.51, 126.15, 126.12, 100.61, 97.50, 91.98, 77.57, 74.68, 74.10, 73.82, 73.56, 73.38, 73.28, 70.55, 69.17, 68.93, 66.24, 62.18, 37.82, 37.79, 29.67, 289.38, 28.11, 28.04; HRMS (MALDI-FTMS) calcd. for C₂₅H₂₉O₈ [M+H]⁺ 457.1862 found 457.1856.

### O-(2-O-benzyl-4,6-O-benzylidene-3-O-levulinyl-D-galactopyranosyl)Trichloroacetimidate (IV)

To a solution of III (188.5 mg, 0.46 mmol) dissolved in 4 ml of DCM was added CCl₃CN (0.46 ml, 4.62 mmol) and DBU (31 μl, 0.21 mmol). After 2 hours at room temperature, the dark solution was concentrated and then purified by flash chromatography Hexanes:EtOAc (2:1) and 1% triethylamine to yield 8 (211 mg, 77%).

¹H(CDCl₃ 500 MHz) δ=7.59-7.34 (m, 10H), 5.61 (s, 1H), 5.45 (dd, J=3.2 Hz, 10.7 Hz, 1H), 4.80-4.72 (m, 2H), 4.60 (d, J=3.3 Hz, 2H), 4.38-4.33 (m, 2H), 4.13-4.10 (dd, J=1.8 Hz, 12.5 Hz, 1H), 4.05 (s, 1H), 2.79-2.72 (m, 2H), 2.65 (m, 2H), 2.16 (s, 3H); ¹³C NMR (125 MHz, CDCl₃) δ=206.43, 177.73, 172.35, 172.27, 138.41, 137.78, 137.63, 137.57, 128.89, 128.85, 128.38, 128.21, 128.03, 127.86, 127.75, 127.67, 127.51, 126.15, 126.12, 100.61, 97.50, 91.98, 77.57, 74.68, 74.10, 73.56, 73.38, 73.28, 70.55, 69.17, 68.93, 66.24, 6218, 37.82 37.79, 29.67, 29.38, 28.11, 28.04.

### 2-Azido-3,4-di-O-benzyl-1-O-(2-O-benzyl-4,6-O-benzylidene-3-O-levulinyl-α-D-galactopyranosyl)-D-ribo-octadeca-6-ene-1-ol (VI)

A solution of trichloroacetimidate IV (150 mg, 0.25 mmol, 1.5 equiv)) and sphingosine derivative V (86 mg, 0.16 mmol) in 2.5 mL of anhydrous THF was added over freshly dried powdered AW-300 molecular sieves and cooled to −20° C. TMSOTf (23 μL, 0.8 equiv) was slowly added to the solution, and the mixture was warmed up to 0° C. in 2.5 hours. The reaction was quenched by addition of Et₃N (0.1 mL), and the mixture was diluted with EtOAc and filtered through Celite. The organic layer was washed with saturated aqueous NaHCO₃ and brine, dried (Na₂SO₄), and concentrated. The residue was purified by column chromatography on silica gel (hexanes:EtOAc 6:1) to furnish VI (57 mg, 46% based on consumed acceptor V) as a syrup, and recover V (18 mg).

¹H NMR (CDCl₃, 400 MHz): δ=7.49-7.23 (m 20H), 5.56-5.45 (m 3H), 5.32 (dd, 1H, J=3.5 Hz, 10.5 Hz), 4.98 (d, 1H, J=3.1 Hz), 4.70-4.51 (m, 6H), 4.38 (m, 1H), 4.13-3.82 (m, 5H), 3.71-3.62 (m, 4H), 2.75-2.40 (m, 6H), 2.08 (s, 3H), 2.06-1.97 (m, 2H), 1.25 (bs, 18H), 0.88 (t, 3H, J=7.0 Hz); ¹³C NMR (125 MHz, CDCl₃) δ=206.30, 172.25, 138.21, 137.93, 137.67, 132.60, 128.89, 128.37, 128.35, 128.33, 129.29, 128.08, 127.27, 128.08, 127.78, 127.73, 127.69, 127.63, 127.60, 127.17, 124.69, 100.65, 98.61, 79.41, 78.95, 74.06, 73.65, 73.41, 73.10, 71.94, 70.79, 69.02, 68.21, 62.41, 61.97, 37.93, 31.89, 29.71-29.32, 28.19, 27.58, 22.66, 14.10; ESI-MS (positive-ion mode): m/z 982.4 [M+Na]⁺.

### 3,4-Di-O-benzyl-1-O-(2-O-benzyl-4,6-O-benzylidene-α-D-galactopyranosyl)-2-hexacosylamino-D-ribo-octadeca-6-ene-1-ol (X)

The azide VI (57 mg, 0.059 mmol) was dissolved in 2.0 mL of anhydrous THF and cooled to 0° C. PMe₃ (0.4 mL of 1.0 M in toluene, 0.4 mmol) was added to the solution, and the reaction was warmed up to room temperature and stirred over night. After almost disappearance of the starting material, 0.8 mL of aq 1 M NaOH was added to the mixture and stirred for 5 hours. CH₂Cl₂ was then added to the solution, and the mixture was washed with brine, dried over Na₂SO₄, and concentrated. The residue was used for the next step without further purification. Hexacosanoic acid (35 mg, 0.088 mmol, 1.5 eq) was suspended in CH₂Cl₂ (2.0 ml), and then DEPBT (26 mg 0.087 mmol, 1.5 eq) and DIEA (15 μL, 1.5 eq) were added. The mixture was vigorously shaken for 1 h to give a clear light yellow solution in which above crude amine mixture VIIIa and VIIIb was added subsequently. The solution was stirred over night at room temperature and then diluted with EtOAc and washed with saturated NaHCO₃ and brine. The organic phase was dried over Na₂SO₄ and concentrated to afford a solid (IXa and IXb, 57 mg), which was dissolved in 2 mL Py-HOAc solution (3:1 v/v, contains 0.30M NH₂NH₂.HOAc) and stirred for 1.5 h at room temperature. After the usual workup similarly as above, the residue was purified by column chromatography on silica gel (hexanes:EtOAc 2:1) to furnish X (40 mg, 56% over 3 steps) as a solid.

¹H NMR (CDCl₃, 400 MHz) δ=7.47-7.23 (m 20H), 5.67 (d, 1H, J=8.6 Hz), 5.51-5.44 (m, 3H), 4.95 (d, 1H, J=2.7 Hz), 4.77-4.49 (m, 6H), 4.40 (m, 1H), 4.21 (d, 1H, J=2.7 Hz), 4.12-4.07 (m, 2H), 3.94-3.58 (m, 8H), 2.45 (m, 2H), 2.08-1.88 (m, 4H), 1.49 (m, 2H), 1.25 (bs, 62H), 0.88 (t, 6H, J=7.0 Hz); ¹³C NMR (CDCl₃, 100 MHz): δ=173.14, 138.50, 138.33, 137.74, 132.57, 129.32, 128.62, 128.40, 128.08, 127.95, 127.85, 126.45, 125.20, 101.37, 99.04, 79.97, 79.22, 76.29, 73.48, 73.41, 71.79, 69.50, 68.86, 68.19, 62.94, 50.26, 36.96, 32.13, 29.91-29.56, 28.14, 27.78, 25.93, 22.90, 14.34; HRMS (MALDI-FTMS) calcd for C₇₈H₁₁₉NO₉Na [M+Na]⁺ 1236.8777, found 1236.8741.

### 3,4-Di-O-benzyl-1-O-(2-O-benzyl-4,6-O-benzylidene-3-O-sulfo-α-D-galactopyranosyl)-2-hexacosylamino-D-ribo-octadeca-6-ene-1-ol, sodium salt (XI)

To a solution of X (40 mg, 0.033 mmol) in Py (2.5 mL) was added SO₃.Py complex (79 mg, 0.5 mmol, 15 eq). The mixture was stirred at room temperature for 2.5 hours. Water solution (2.5 mL) of NaHCO₃ (62 mg) was added to quench the reaction. The reaction mixture was diluted with CH₂Cl₂, and washed with brine, dried (Na₂SO₄), and concentrated. The residue was purified by column chromatography on silica gel (CH₂Cl₂:MeOH 15:1) to give XI (39 mg, 90%) as a solid.

¹H NMR (CDCl₃/CD₃OD 1:1, 400 MHz) δ=7.87 (d, 1H, J=8.9 Hz), 7.58-7.17 (m, 20H), 5.59 (s, 1H), 5.43 (m, 2H), 4.96 (m, 3H), 4.82 (m, 1H), 4.73 (d, 1H, J=2.3 Hz), 4.62-4.58 (m, 2H), 4.52-4.44 (m, 2H), 4.19-3.99 (m, 5H), 3.78 (bs, 2H), 3.66 (bs, 1H), 3.56 (m, 1H), 2.47 (m, 1H), 2.34 (m, 1H), 2.13 (t, 2H, J=7.0 Hz), 2.01 (m, 2H), 1.54 (bs, 2H), 1.27 (bs, 62H), 0.89 (t, 6H, J=7.0 Hz); ¹³C NMR (CDCl₃/CD₃OD 1:1, 100 MHz): δ=173.92, 138.30, 137.66, 137.57, 131.42, 128.43, 128.01-127.03, 125.95, 125.66, 100.59, 98.99, 80.16, 79.75, 75.04, 74.84, 73.97, 73.60, 73.49, 71.09, 68.74, 67.00, 62.70, 49.71, 49.62, 31.56, 29.29-29.02, 27.01, 25.59, 22.27, 13.44; HRMS (MALDI-FTMS) calcd for C₇₈H₁₁₈NO₁₂SNaK [M+K]⁺ 1354.7909, found 1354.7933.

### 2-Hexacosylamino-1-O-(3-O-sulfo-α-D-galactopyranosyl)-D-ribo-1,3,4-octadecantriol, Sodium Salt (4)

XI (39 mg, 0.030 mmol) was dissolved in HOAc-MeOH (1:1 v/v, 6 mL). 80 mg of palladium black was added and the reaction solution was saturated with hydrogen by a balloon. After stirring at room temperature for 20 hours, the catalyst was removed by filtration over Celite and washed with CH₂Cl₂/MeOH (1:1) thoroughly. Evaporation of the solvent gave a residue which was dissolved in CH₂Cl₂/MeOH (1:1) mixed solvent again and then saturated NaHCO₃ (3 mL) was added to stir at room temperature for half an hour. After removal of the solvent, the residue was purified by column chromatography on silica gel (CH₂Cl₂:MeOH 6:1) to give 4 (24 mg, 83%) as a light yellow solid.

¹H NMR (CDCl₃/CD₃OD 1:1, 400 MHz) δ=4.95 (d, 1H, J=3.5 Hz), 4.49 (dd, 1H, J=2.7 Hz, 10.2 Hz), 4.35 (m, 1H), 4.17 (m, 1H), 4.02 (dd, 1H, J=2.7 Hz, 9.8 Hz), 3.88-3.85 (m, 2H), 3.80-3.72 (m, 4H), 3.69-3.65 (m, 2H), 3.61-3.57 (m, 1H), 2.24 (t, 2H, J=7.4 Hz), 1.59 (m, 4H), 1.27 (bs, 68H), 0.89 (t, 6H, J=7.0 Hz); ¹³C NMR (CDCl₃/CD₃OD 1:1, 100 MHz): δ=174.31, 99.08, 77.57, 73.42, 71.64, 70.44, 67.81, 67.19, 66.48, 61.28, 49.90, 35.89, 31.51, 31.32, 29.29-28.94, 25.53, 22.22, 13.34; HRMS (MALDI-FTMS) calcd for C₅₀H₉₈NO₁₂SNa₂ [M+Na]⁺ 982.6599, found 982.6585.

### Synthesis Scheme

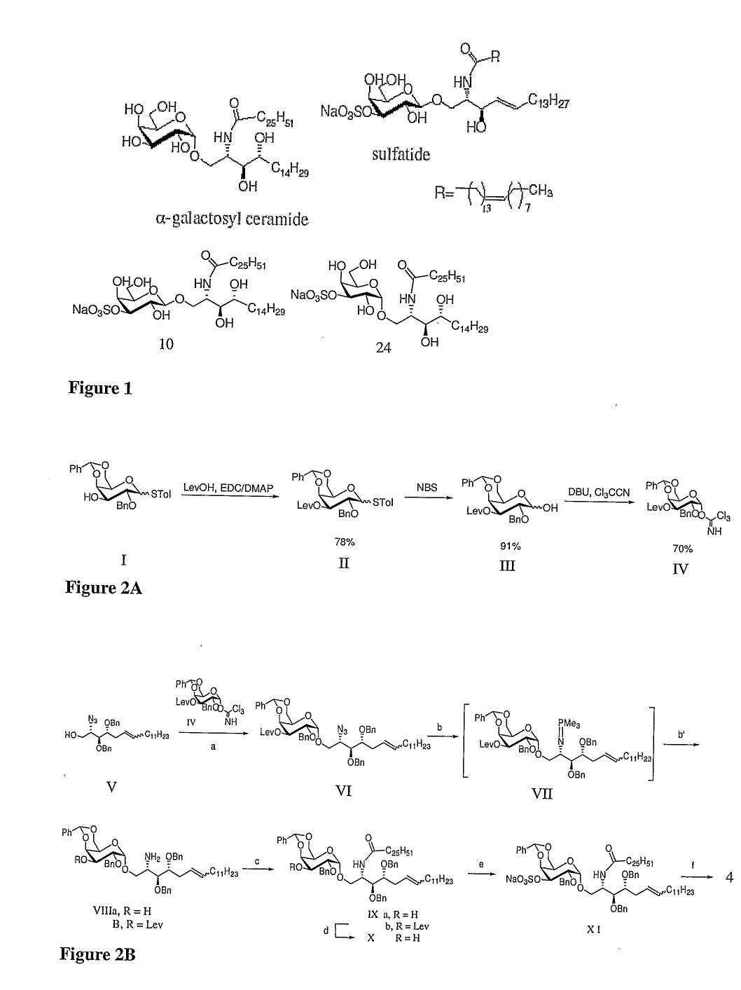

Sulfatide and α-galactosyl ceramide have similar structures and possess immunostimulatory and immunomodulatory activity, when presented to T cells via CD1. In order to determine whether hybrid molecules of sulfatide and α-galactosyl ceramide, which are sulfate derivatives 3-O-sulfo-α/β-galactosylceramides 10 and 4 (FIG. 1), have comparable activity, the molecules were synthesized and evaluated for immunostimulatory activity.

For the synthesis of 3-O-sulfo-a-galactosylceramides 4, selective sulfation at 3″ OH of the galactose moiety is a key step. Typically, regioselective sulfation of the 3-hydroxyl of the sugar ring utilizes dibutylstannylene acetals as activated intermediates, however, this method can only be applied to β-galactosides; for α-galactosides, the dibutylstannylene acetal can form a complex between the 2-hydroxyl and the anomeric oxygen to give the 2″-O-derivative by reaction with an electrophile.

In order to address this, a 3″-lev and 2″-benzyl-4″,6″-benzylidene protected trichloroacetimidate donor IV (FIG. 2). The temporary protecting group Lev, can be selectively removed after glycosylation in the presence of hydrazine. The benzyl and benzylidene groups at 2, 4, 6 positions direct the next α-glycosidic bond formation (Figueroa-Perez, S. et al Carbohydrate Res. 2000, 328, 95; Plettenburg, O. et al. J. Org. Chem. 2002, 67, 4559).

As shown in FIG. 2A, the preparation of IV started with the known thioglycoside I in 50% yield over three steps. The sphingosine building block V was employed in this synthesis, with donor IV coupled to acceptor V in the presence of TMSOTf, used as promoter to give α-glycoside VI, in a moderate yield.

A staudinger reduction of VI with PMe₃, in NaOH solution was used to hydrolyze the imino-phosphorane intermediate VII, however, the Lev group cannot survive under this conditions and approximate 50% of the Lev group was cleaved to give an amine mixture of VIIIa and VIIIb (1:1) determined by ¹H NMR. Since VIIIa possesses a free C-3 hydroxyl, it is crucial to choose a selective coupling reagent in the condensation between amine VIIIa and the fatty acid.

Since DEPBT [3-(diethoxyphosphoryloxy)-(1,2,3)-benzotriazin-4(3H)-one] can selectively form an amide bond in the presence of unprotected hydroxyl groups, it was used in the reaction mixture with VIIIa, VIIIb, and hexacosanoic acid to give IXa and IXb, followed by deprotection of the remaining Lev groups using hydrazine to provide the desired galactosyl ceramide X in 56% yield over 3 steps. Treating the 3″-OH free glycolipid X with Py-SO₃ led to the sulfate derivative XI in high yield, which gave compound 4 upon hydrogenation with palladium black and neutralization with NaHCO₃ (aqueous solution) in 78% yield (FIG. 2B).

### Example 2

### Synthesis of Analogs of Glycolipid α-Galactosyl Ceramide

### 3-O-sulfo-β-galactosylceramide

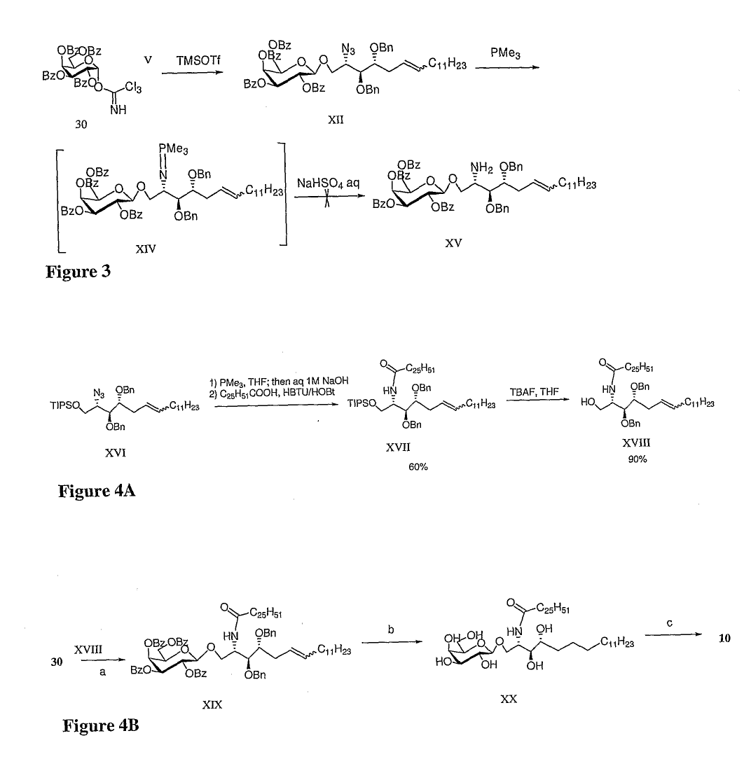

For the synthesis of 10, perbenzoylated trichloroacetimidate donor 40 is used in the glycosylation of the sphingosine acceptor V to yield a β-galactosyl ceramide derivative XII (FIG. 3). After the Staudinger reduction of XII, a complex mixture was produced, with no isolation of the amine XV. Since the perbenzoylated galactosyl ceramide is sensitive to basic conditions, a NaHSO₄ solution instead of a NaOH solution was used for the reduction work-up procedure to decompose the imino-phosphorane intermediate XIV. However, hydrolyzation of XIV into XV was very slow, and in turn, the longer reaction time led to the degradation of glycosidic bond which attributed to complicated product formation.

### Example 3

### Synthesis of Analogs of Glycolipid α-Galactosyl Ceramide

### 3-O-sulfo-β-galactosylceramide

Another synthetic strategy was used to synthesize 3-O-sulfo-β-galactosylceramide. In this strategy, the azide was first reduced, and the fatty acid coupled, prior to the glycosylation step (FIG. 4A). Compound XVIII was prepared from the sphingosine derivative XVI (Plettenburg, O. et al. J. Org. Chem. 2002, 67, 4559) in 54% yield over 2 steps.

Using TMSOTf as a promoter, the ceramide acceptor XVIII was reacted with donor 40 to give the β-glycoside XIX in 54% yield. After debenzyolation and hydrogenation of XIX, the β-galactosyl ceramide XX was obtained in quantitative yield. XX was finally sulfated by Bu₂SnO/Me₃N.SO₃ and subsequently neutralized by NaHCO₃ to give the product 10, in 80% yield (FIG. 4B) (Compostella, F et al. Tetrahedron 2002, 58, 8703).

### Example 4

### Recognition of Glycolipids by the Human NKT Cell Line Results in Cytokine Secretion

### Materials and Methods

### Glycolipids

α-GalCer was obtained as described [Plettenburg, O., et al. (2002) J. Org. Chem. 67, 4559-64]. The intermediates 29, 36 and 40 (FIGS. 3 and 4), were obtained as described [Plettenburg, O., et al. (2002) supra; Williams, L., et al. (1996) Tetrahedron 52, 11673-11694; Deng, S. Y., Bet al. (1999) J. Org. Chem. 64, 7265-7266]. The compounds 5, 6, 19, 30, 33, 37, and 41 (FIGS. 3 and 4) were obtained as described hereinbelow. The remaining compounds, except 19, 10 and 4, and their intermediates were obtained as described hereinabove.

### Sphingosine Acceptor

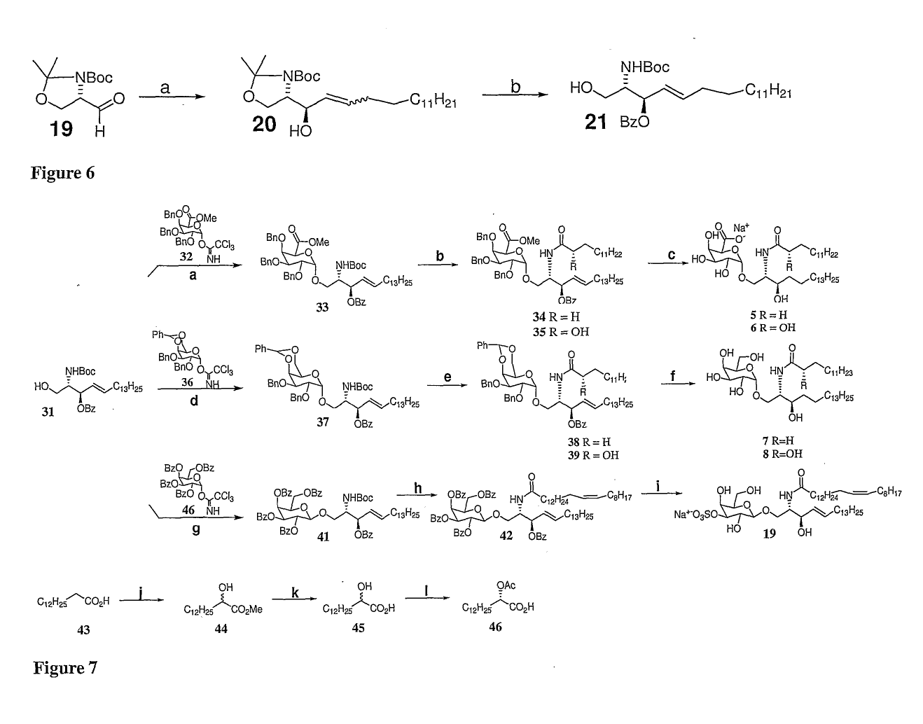

The synthesis scheme for the sphingosine acceptor (30) is shown in FIG. 6. Compound 29 (3.31 g, 13.5 mmol) (Williams, L., et al. supra) was dissolved in 70 ml of dry THF. The solution was cooled to −40° C. and vinyl grignard solution (31 ml of a 1 M solution in THF) was added via a dropping funnel over a period of 1 hr. The temperature was kept between −20° C. and −40° C. The reaction mixture was allowed to warm to room temperature and stirred for another hr. The reaction was quenched by addition of 60 ml of saturated (NH₄)₂SO₄ solution and evaporated to dryness. The residue was diluted with water and extracted with ethyl acetate (3×). The combined organic layer was extracted with brine, dried over MgSO₄ and evaporated to give a yellow oil. Column chromatography (Hex:EtOAc 3:1) yielded the syn diastereomer (2.11 g, 8.2 mmol, anti/syn=3.5:1) in 61% yield. Then the syn diastereomer (300 mg, 1.16 mmol) was dissolved in 1 ml of dry dichloromethane in a two-necked flask equipped with a reflux condenser under argon. 486 mg (3.48 mmol) of pentadecene was added via a syringe. A solution of 20 mg (2 mol %) of Grubb's second generation catalyst (purchased from Strem Chemicals) in 1 ml of dichloromethane was added and the solution was heated under rapid reflux for 40 hr. The reaction mixture was evaporated and then directly chromatographed (Hex:EtOAc 6:1) which yielded (0.82 mmol, 71%) of the desired product.

### Synthesis of Glycolipids

The synthesis scheme is shown in FIG. 4. A solution of trichloroacetimidate 32 (160.4 mg, 0.258 mmol) and sphingosine acceptor 31 (100 mg, 0.198 mmol) in 4 ml of anhydrous Et₂O and 2 ml of anhydrous THF was added over freshly dried 4 Å molecular sieves and cooled to −50° C. Trimethylsilylmethyl trifluoromethanesulfonate (TMSOTf) (3.33 mg, 0.0198 mmol) was added and the mixture stirred at −50° C. for 1 hour. The mixture was allowed to warm to −20° C. and another 3.33 mg of TMSOTf was added. The mixture was then slowly allowed to warm to room temperature and stirred for 3 hour. The solution was then diluted with EtOAc and filtered over celite. The organic layer was washed with saturated aqueous NaHCO₃ and brine, dried (MgSO₄), and concentrated. The residue was purified by column chromatography on silica gel (toluene:EtOAc 12:1) to give 128 mg (67.5%, 0.134 mmol) of 33.

Compound 34 (36 mg, 0.03 mmol), dissolved in 6 ml of EtOAc, was added to 36 mg of 20 wt % palladium hydroxide in 1 ml of EtOAc and saturated with hydrogen. The reaction vessel was purged with hydrogen, and the mixture was stirred at room temperature overnight. The reaction mixture was filtered and the solvent was evaporated. The above hydrogenated compound was dissolved in 2 ml THF, 1 ml water, and 1 ml methanol. LiOH (9 mg, 0.14 mmol) was added to the solution and the reaction was stirred at room temperature for four hours. 100 mg of Na₂CO₃ was added and the mixture stirred for 30 minutes. The solvent was evaporated and the remaining residue was purified on silica gel by column chromatography (CH₂Cl₂:MeOH 4:1) to give 7.8 mg of 1 (38%, 0.0114 mmol, 2 steps).

After deprotection of compound 42 (14 mg, 0.017 mmol), Bu₂SnO (6.5 mg, 0.0259 mmol) dissolved in 1 ml of MeOH was added. The mixture was refluxed under argon for 2 h. After evaporation of the solvent, Me₃N.SO₃ (5 mg, 0.035 mmol) dissolved in 1 ml THF was added and the reaction was allowed to proceed at room temperature for 6 hours. The solvent was then removed under reduce pressure and the residue dissolved in CHCl₃/MeOH 1:1 (1 mL) and loaded onto an ion exchange column (Dowex 50×8 Na+ form). After elution with CHCl₃/MeOH 1:1, the mixture was concentrated and purified by column chromatography (CH₂Cl₂:MeOH 5:1) to give 18 (14.4 mg, 95%).

### 1.2 Hybridoma Assay

CD1d reactive T cell hybridomas with an invariant Vα14i T cell antigen receptor a: chain were used, as described (Sidobre, S., et al. (2004) Proc. Natl. Acad. Sci. USA 101, 12254-12259). T cell hybridomas were stimulated with the indicated glycolipids that were added either to plates coated with soluble CD1d, or with CD1d transfected A20 B lymphoma cells, as described (Elewaut, D., et al. (2003) J. Exp. Med. 198, 1133-1146). As a measure of T cell activation, IL-2 release into the tissue culture medium was measured after 16 hours culture by an ELISA assay.

### Generation of Vα42i Human NKT Cell Line

Human NKT cell lines, expressing the Vα24i T cell receptor as well as CD161, were generated as follows: Anti-CD161 monoclonal antibodies, and anti-CD14 monoclonal antibodies, each coupled to magnetic beads (Miltenyi biotec, Auburn, Calif.), were used sequentially to isolate CD161⁺ cells and CD14⁺ cells from leukopaks. Immature dendritic cells were generated from the CD14⁺ cells after a two-day incubation in the presence of 300 U/ml GM-CSF (R&D systems, Minneapolis, Minn.) and 100 U/ml IL-4 (R&D systems, Minneapolis, Minn.). Following irradiation with 2000 rads, the immature dendritic cells were co-cultured with syngeneic CD161⁺ cells in the presence of 100 ng/ml of alpha-galactosylceramide and 10 IU/ml of IL-2 (Invitrogen, Carlsbad Calif.) for 10 to 14 days. After stimulating the CD161⁺ cells a second time with alpha-galactosylceramide-pulsed, irradiated immature dendritic cells, NKT cell lines were shown by flow cytometry to express both CD161⁺ and V 24i TCR (99% purity).

### In Vitro Cytokine Secretion Assay Using Human NKT Cell Lines

IFN-γ and IL-4 secretion by the Vα 24i human NKT cell line was determined by ELISA (BD Pharmingen, San Diego, Calif.) after culture for 16 hours. For these assays, 1×10⁵ Vα 24i human NKT cells were co-cultured with 4×10⁵ irradiated, immature CD14⁺ dendritic cells, in the presence of the glycolipid compounds at 10 μg/ml in a 96-well flat-bottom plate.

### Results

In order to test whether glycolipids of bacterial origin (5, 6, 8, 17) (represented in FIG. 5), or analogs thereof, which comprise structures similar to α-GalCer either at the sugar or lipid moiety, activate NKT cells through CD1d, the glycolipids were synthesized and assayed. Analogs 7 and 8 (FIG. 5) were prepared, and used to probe the effect of the carboxyl group on the sugar and the α-hydroxyl group on the lipid. Compounds 19, 10 and 4 contain a 3′-sulfate group with an α or β-glycosidic linkage. 20-23 were prepared to probe the effect of the 2′-modification of α-GalCer. Analogs of α-GalCer with modification of the lipid moiety were also prepared to probe their interaction with CD1d and the subsequent effect on NKT cell activation.

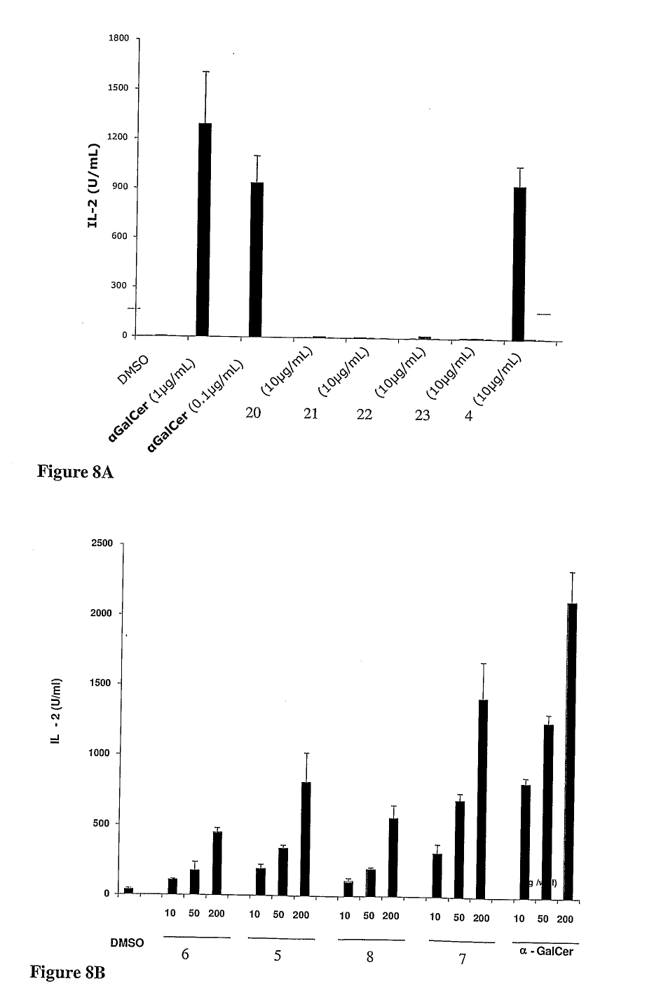

Mouse Vα14i NKT cells immortalized by cell fusion provided a convenient method for assaying the ability of the synthetic glycolipids to activate T cells. As shown in FIG. 8a, the 3-O-sulfo-α-GalCer, 4, stimulated significant IL-2 release from the hybridomas when used at 10 μg/ml. Dose response curves indicated, however, that this compound was somewhat less active than αGalCer (data not shown) in this model. By contrast, every modification of the 2 OH position of the galactose (compounds 10-13) that were tested abolished all biological activity. These data indicate that the Vα14i NKT cell response to glycolipids apparently is more sensitive to modifications of the 2 than to the 3 position.

B. burgdorferi glycolipids (17-18) and compounds 24 and 25 were moderately active in the 1.2 hybridoma assay. However IL-2 secretion could only be detected when large quantities of the glycolipids were used to stimulate the hybridoma cells (data not shown).

CD1d coated plates were used to assay response of the hybridomas to the Sphingomonas glycolipids (FIG. 8b). A substantial level of IL-2 secretion can be observed for all compounds. The structure of the sugar head group significantly affected the activation of the hybridomas. α-GalCer and the galactose analogue 7, consistently solicited greater IL-2 secretion when compared to the galacturonic acid derivatives. Also affecting activity was the (S)-2-hydroxy of the fatty amide tail. A fully saturated tail was more greatly favored, suggesting that the α-hydroxyl group is not optimal. In fact the (S)-2-hydroxy appeared to have a greater affect on activity as compound to compound 8, a galactose analogue, that was less able to activate IL-2 secretion when compared with 5, the galacturonic acid compound without the α-hydroxyl fatty amide. Though 7 and 8 are not known to be natural products, both could be precursors to compounds 5 and 6.

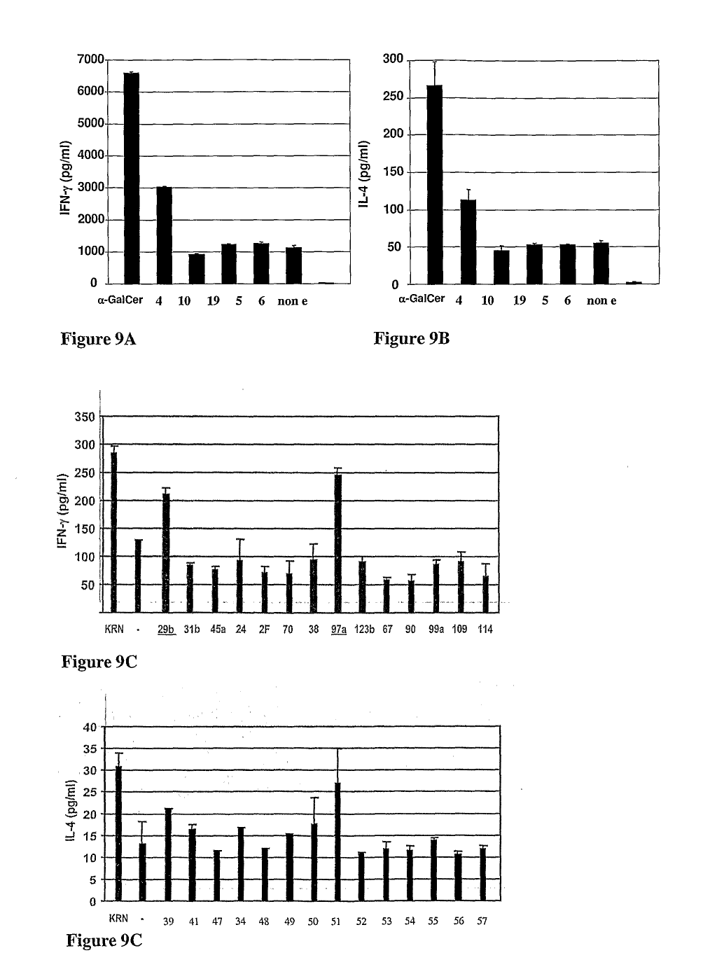

IFN-γ and IL-4 secretion from a Vα24i NKT cell line were assessed, after stimulation with irradiated, syngeneic CD14⁺ immature dendritic cells in the presence of 10 μg/ml of the glycolipids and 10 IU/ml of IL-2 (FIG. 9a). Stimulation of the NKT cell line by each glycolipid compound resulted in significant IFN-γ and IL-4 secretion, when compared to the negative control. While greater IFN-γ and IL-4 secretion was observed after stimulation by the potent NKT cell agonist, α-GalCer, secretion of IFN-γ and IL-4 by NKT cells stimulated by 1-10 μg/ml of 3-O-sulfo-α-galactosylceramide was approximately half that of α-GalCer, but twice that induced by the other glycolipids. β-linked sulfatides 19 and 10 were also observed to elicit both IFN-γ and IL-4 production. In fact, the level of cytokine secretion was comparable to the GSLs.

As illustrated in FIGS. 8C and 8D, interferon-γ secretion by human NKT cells in response to glycolipid presentation by CD14⁺ DCs, was superior when the glycolipid was 3-sulfo-α-GalCer 4, as compared to α-GalCer, at a concentration of 10-20 μg/mL. Compound 4 efficiently stimulated IL-4 and IFN-γ secretion, indicating that the modification of the 3″-OH position of the galactose moiety with sulfate was useful in NKT cells stimulation.

NKT cells activation was sensitive to the configuration of the anomeric carbon of glycolipid antigen molecules. 3-sulfo-β-GalCer 10 had minimal to no affinity for NKT lymphocytes due to the β-linkage of glycosidic bond, indicating that the α-linkage of the glycoside was essential for CD1 antigen binding.

Other α-GalCer analogs with an acetyl side chain or a shortened backbone were also tested and some activity was also observed (FIGS. 8C and 8D, and data not shown).

### Example 5

### Human NKT Cell Lines Bind to Glycolipids in the Context of CD1d

### Materials and Methods

In Vitro CD1d-Dimer Assay Using a Human NKT Cell Line

One mg of soluble divalent human CD1d-IgG1 fusion protein (human CD1d-IgG1 dimers, BD Pharmingen) were incubated overnight with 10 M of each glycolipid at 32° C. and at neutral pH according to the manufacturer's protocol. The glycolipid-loaded CD1d-IgG1 dimers were incubated with human NKT cells at 4° C. for 60 minutes, followed by incubation with PE-coupled anti-mouse IgG1 mAb (A85-1). The cells were also surface stained with a PerCP-coupled anti-CD3 mAb (BD Pharmingen, San Diego, Calif.).

### Results

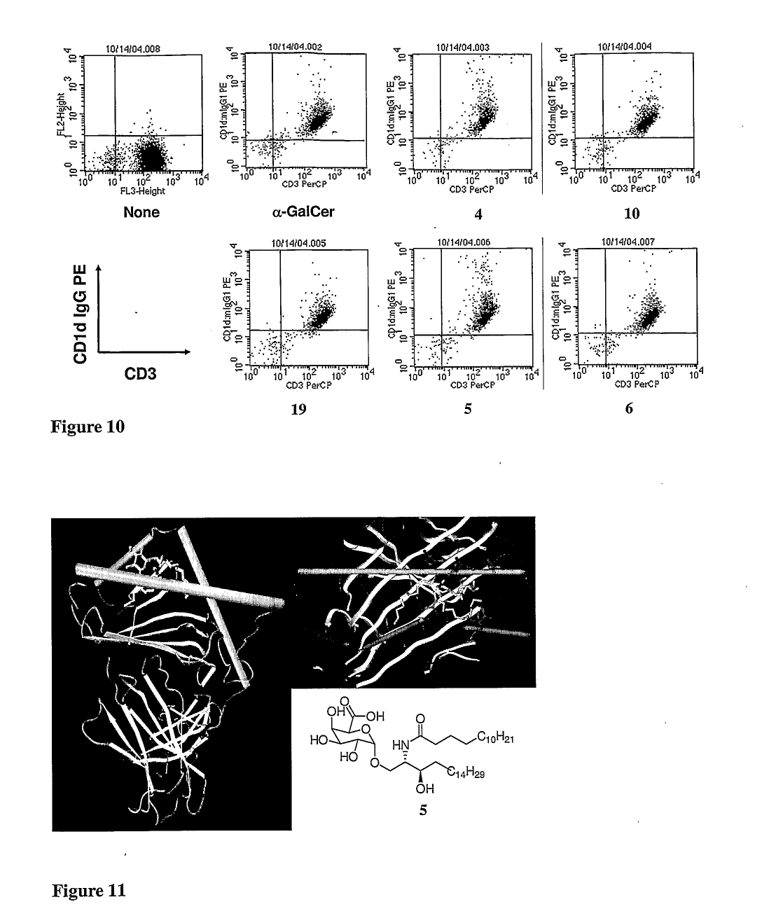

Although glycolipids stimulated the NKT cell line, it does not necessarily follow that the glycolipids were presented by CD1d molecules and were capable of triggering the Vα24i T cell receptor expressed by the NKT cells. Therefore, in order to demonstrate glycolipid antigen reactivity to the Vα24i T cell receptor at the single cell level, a human NKT cell line with human CD1d dimers loaded with different glycolipids was stained, and unloaded CD1d dimers were used as a negative control. Each glycolipid-loaded dimer nearly universally stained the human NKT cells, while the unloaded dimer did not stain these cells (FIG. 10).

### Example 6

### Computer Modeling of GSL Complexed to MCD1d

### Materials and Methods

### Model Generation

A model of GSL 1 complexed with the crystal structure of mCD1d (Zeng, Z., et al. (1997) Science 277, 339-45) was generated by Autodock 3.0 (Morris, G. M., et al. (1998) J. Comput. Chem. 19, 1639-1662).

### Results

To further understand the interaction of bacterial glycolipid 1 with CD1d, a model of GSL 1 complexed with mCD1d was generated, and is shown in FIG. 11. According to the model, the fatty acyl chain extended into the F′ pocket and the sphingosine chain toward the A′ pocket. This allowed for the polar head group to be oriented such that it was exposed for recognition by a T cell antigen receptor. Numerous favorable contacts could be observed between mCD1d and the glycosphingolipid. Among them, possible hydrogen bonding included interactions between the carboxylate of the sugar and the backbone carbonyl of Asp80, and the amide nitrogen of the fatty acid tail with the Asp80 sidechain.

While it was thought that mCD1d to be somewhat accommodating in terms of lipid tail length on NKT cell reactivity, changes in the lipid length, composition, or addition of an α-hydroxyl group on the fatty acid, as seen in FIG. 11, could cause a slight shift in orientation of the sugar and thereby affect CD1d/glycolipid complex recognition by the T-cell receptor. Substitution of galacturonic acid for galactose may produce similar results. The perturbation caused by having the 6-OH oxidized to a carboxylic acid caused only moderate changes in NKT cell reactivity, thus the model provides an effective means for designing additional ligands.

### Example 7

### Synthesis of Analogs of Glycolipid α-Galactosyl Ceramide

A number of glycolipids were synthesized and tested for NKT cell activation. A synthetic scheme is provided in scheme 1 below:

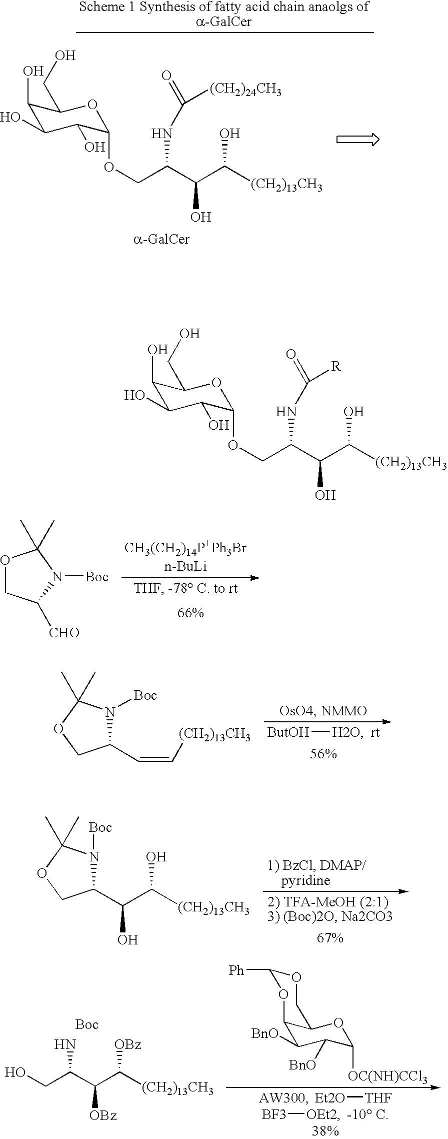

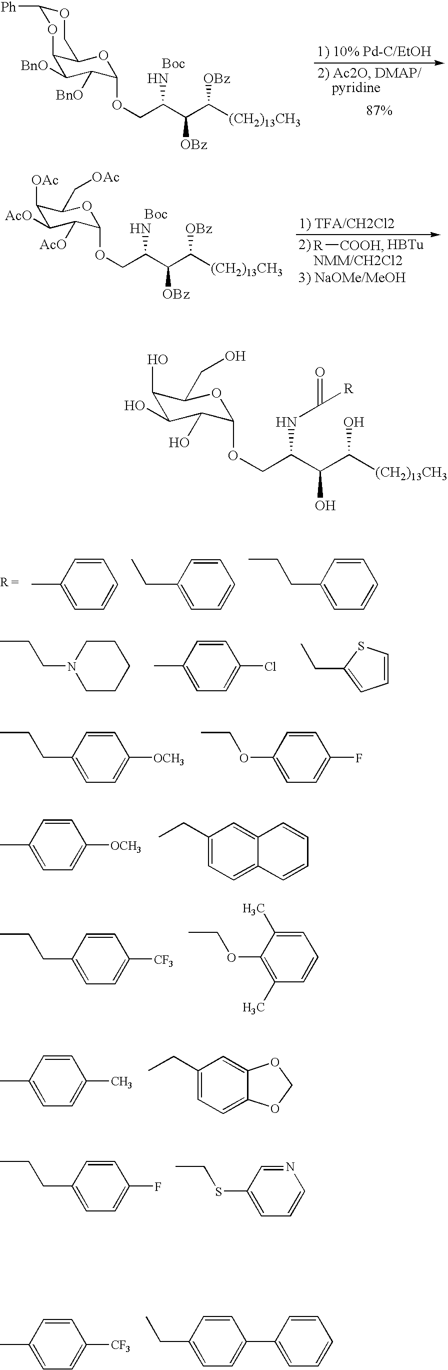

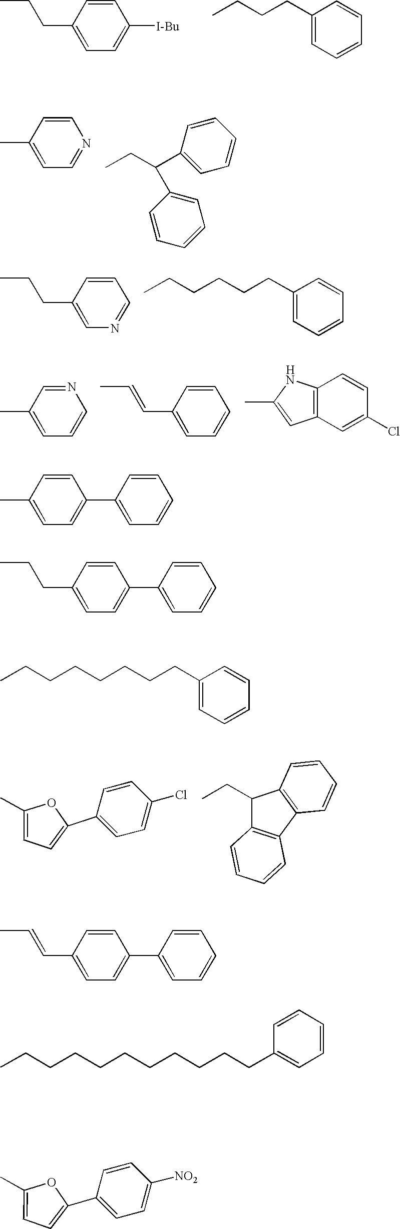

Other compounds were synthesized as described in Xing G W et al. Bioorg Med. Chem. 2005 Apr. 15; 13(8):2907-16; and Wu D. et al., Proc Natl Acad Sci USA. 2005 Feb. 1; 102(5):1351-6.

All chemicals were purchased as reagent grade and used without further purification. Dichloromethane (CH₂Cl₂) were distilled over calcium hydride. Tetrahydrofuran (THF) and ether were distilled over sodium metal/benzophenone ketyl. Anhydrous N,N-dimethylformamide (DMF) was purchased from Aldrich. Molecular sieves (MS) for glycosylation were AW300 (Aldrich) and activated by flame. Reactions were monitored with analytical thin layer chromatography (TLC) in EM silica gel 60 F254 plates and visualized under UV (254 nm) and/or staining with acidic ceric ammonium molybdate or ninhydrin. Flash column chromatography was performed on silica gel 60 Geduran (35-75 um, EM Science). ¹H NMR spectra were recorded on a Bruker DRX-500 (500 MHz) spectrometer or a Bruker DRX-600 (600 MHz) spectrometer at 20° C. Chemical shifts (δ ppm) were assigned according to the internal standard signal of tetramethylsilane in CDCl₃ (δ=0 ppm). ¹³C NMR spectra were obtained using Attached Proton Test (APT) on a Bruker DRX-500 (125 MHz) spectrometer Bruker DRX-600 (150 MHz) spectrometer and were reported in 6 ppm scale using the signal of CDCl₃ (δ=77.00 ppm) for calibration. Coupling constants (J) are reported in Hz. Splitting patterns are described by using the following abbreviations: s, singlet; brs, broad singlet; d, doublet; t, triplet; q, quartet; m, multiplet. ¹H NMR spectra are reported in this order: chemical shift; number(s) of proton; multiplicity; coupling constant(s).

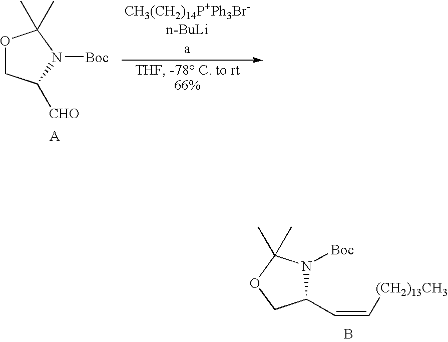

To a stirred solution of Wittig reagent a (6.1 g, 11 mmol), prepared from 1-bromopentadecane and triphenylphosphine refluxed in toluene for 5 days, in THF (30 mL) was added n-BuLi (1.6 mol/L in hexane, 6.4 mL, 10 mmol) dropwise at −78° C., then the solution was stirred for 1 h at room temperature. After 1 h the solution was cooled to −78° C. and Garner's aldehyde A (2.1 g, 9.2 mmol) in THF (20 mL) was added. After stirring for 1 h at room temperature, the solution was poured into ice-water and extracted with AcOEt. The organic layer was washed with brine, dried with MgSO₄, and evaporated to dryness. The residue was purified by flash column chromatography on silica gel (toluene 100%) to give B (2.6 g, 66%) as a pale yellow oil. ¹H NMR (600 MHz, CDCl₃) δ 5.36-5.52 (2H, m), 4.51-4.75 (1H, m), 4.05 (1H, dd, J=6.3 Hz, 8.6 Hz), 3.63 (1H, dd, J=3.3 Hz, 8.6 Hz), 1.94-2.21 (2H, m), 1.20-1.66 (39H, m), 0.88 (1H, t, J=6.9 Hz). ¹³C NMR (150 MHz, CDCl₃) δ 151.97, 131.98 (brs), 130.70 (brs), 130.28 (brs), 129.36 (brs), 93.94 (brs), 93.38 (brs), 79.69 (brs), 69.04, 54.55, 31.90, 29.72, 29.67, 29.65, 29.63, 29.59, 29.49, 29.33, 29.29, 28.46, 22.66, 14.08. HRMS (ESI-TOF) for C₂₆H₄₉NO₃Na [M+Na]⁺ calcd 446.3604, found 446.3602.

Synthesis of Compound C:

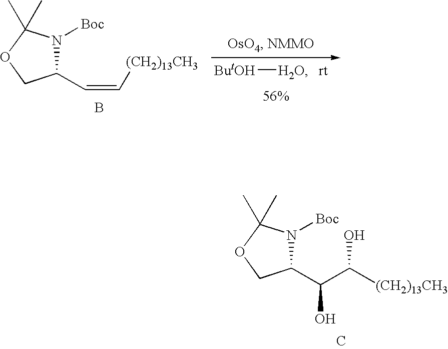

To a stirred solution of B (2.6 g, 6.0 mmol) and 1-methylmorpholine N-oxide (1.1 g, 9.0 mmol) in Bu^{t}OH and H₂O (1:1, 30 mL), OsO₄ (2.5 w/v in Bu^{t}OH, 3.1 mL) was added at room temperature. The solution was stirred overnight and quenched with Na₂SO₃ aq. The solution was extracted 2 times with AcOEt, washed with brine, dried with MgSO₄, and evaporated to dryness. The residue was purified by flash column chromatography on silica gel (CHCl₃ to CHCl₃:MeOH 20:1) to give C (1.6 g, 56%) as a white solid. ¹H NMR (600 MHz, CDCl₃) δ 4.13-4.21 (2H, m), 3.95-4.03 (1H, m), 3.55-3.65 (1H, m), 3.29-3.42 (2H, m), 1.38-1.68 (17H, m), 1.21-1.38 (24H, m), 0.88 (3H, t, J=7.1 Hz). ¹³C NMR (125 MHz, CDCl₃) δ: 153.93, 93.97, 81.35, 74.89, 73.85, 65.26, 59.41, 32.27, 31.87, 29.65, 29.63, 29.59, 29.31, 28.31, 26.80, 26.18, 23.94, 22.64, 14.07. HRMS (ESI-TOF) for C₂₆H₅₁NO₅₁Na⁺ [M+Na]⁺ calcd 480.3659, found 480.3659.

Synthesis of Compound D:

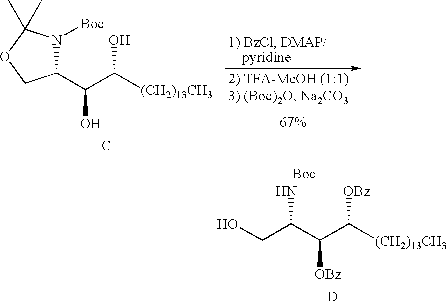

To a stirred solution of C (328 mg, 0.72 mmol) and DMAP (cat.) in pyridine (5 mL) was added BzCl (0.20 mL, 1.8 mmol) and stirred at room temperature overnight. The solution was added Sat. NaHCO₃ aq., extracted with AcOEt, washed with brine, dried with MgSO₄, and evaporated to dryness. The residue was purified by flash column chromatography on silica gel (Hex.: AcOEt 10:1) to give dibenzoylated product (471 mg) as a colorless oil. To a stirred solution of this compound in dry MeOH (5 mL) was added TFA (10 mL) dropwise at 0° C. After 2 h, the solution was evaporated to dryness and co-evaporated with toluene 3 times. The residue was dissolved in dioxane (15 mL) and Sat. NaHCO₃ aq. (15 mL). To a stirred solution, Na₂CO₃ (155 mg) and Boc₂O (320 mg, 1.5 mmol) were added and stirred overnight. This solution was extracted with AcOEt, washed brine, dried with MgSO₄ and evaporated to dryness. The residue was purified by flash column chromatography on silica gel (Hex.: AcOEt 5:1) to give D (301 mg, 67% over 3 steps) as a colorless oil. ¹H NMR (500 MHz, CDCl₃) δ: 8.05 (2H, d, J=7.2 Hz), 7.95 (2H, d, J=7.1 Hz), 7.63 (1H, t, J=7.5 Hz), 7.46-7.54 (3H, m), 7.38 (2H, t, J=7.5 Hz), 5.50 (1H, d, J=9.6 Hz), 5.40 (1H, dd, J=2.4 Hz, 9.3 Hz), 5.33 (1H, d, J=9.5 Hz), 4.00-4.07 (1H, m), 3.64-3.67 (2H, m), 2.55-2.65 (1H, m), 1.96-2.10 (2H, m), 1.48 (9H, s), 1.20-1.45 (24H, m), 0.88 (3H, t, J=7.0 Hz). ¹³C NMR (125 MHz, CDCl₃) δ: 167.03, 166.15, 155.54, 133.73, 133.04, 129.95, 129.64, 129.15, 128.75, 128.63, 128.36, 80.00, 73.81, 73.72, 60.40, 51.46, 31.90, 29.67, 29.65, 29.63, 29.59, 29.53, 29.51, 29.34, 28.30, 25.72, 22.67, 14.11. HRMS (ESI-TOF) for C₃₇H₅₅NO₇Na⁺ [M+Na]⁺ calcd 648.3871, found 648.3866.

Synthesis of Compound E:

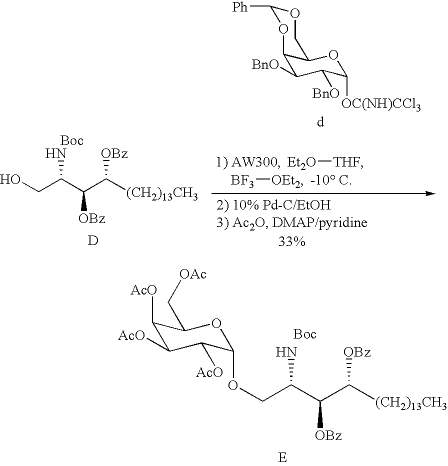

To a stirred solution of D (1.5 g, 2.5 mmol), d (1.8 g, 3.0 mmol) and AW300 (2.0 g) in Et₂O-THF (7:1, 34 mL) was cooled to −40° C. and added BF₃.OEt₂ (0.63 mL, 5 mmol). The solution was stirred for 4 h at ambient temperature, and warmed to room temperature. The solution was filtered, added sat. NaHCO₃ aq. and extracted with AcOEt. The organic layer was dried with MgSO₄ and evaporated to dryness. The residue was purified by flash column chromatography on silica gel (Hex.: AcOEt 5:1) to give a coupled product (980 mg, 38%) as a colorless oil.

To a stirred solution of this compound (980 mg, 0.95 mmol) in EtOH (30 mL) was added 10% Pd—C (490 mg) and stirred vigorously under H₂ atmosphere overnight. The solution was filtered and concentrated to dryness. The residue and DMAP (cat.) were dissolved with pyridine (10 mL) and AC₂O (10 mL), stirred at room temperature overnight. The solution was concentrated, dissolved with AcOEt, washed with brine and concentrated to dryness. The residue was purified by flash column chromatography on silica gel (Hex.: AcOEt 2:1) to give E (790 mg, 87%) as a colorless oil. ¹H NMR (600 MHz, CDCl₃) δ: 8.00 (2H, d, J=7.3 Hz), 7.93 (2H, d, J=7.5 Hz), 7.60 (1H, t, J=7.4 Hz), 7.52 (1H, t, J=7.4 Hz), 7.47 (2H, t, J=7.8 Hz), 7.37 (2H, t, J=7.7 Hz), 5.66 (1H, dd, J=2.3 Hz, 9.6 Hz), 5.41-5.48 (2H, m), 5.29-5.33 (2H, m), 5.16 (1H, dd, J=3.6 Hz, 10.9 Hz), 4.82 (1H, d, J=3.5 Hz), 4.26 (1H, t, J=9.8 Hz), 4.17 (1H, t, J=6.7 Hz), 4.06 (1H, dd, J=6.1 Hz, 11.3 Hz), 4.00 (1H, dd, J=7.1 Hz, 11.3 Hz), 3.78 (1H, dd, J=2.4 Hz, 10.7 Hz), 3.49 (1H, dd, J=2.4 Hz, 10.7 Hz), 2.10 (3H, s), 2.02 (3H, s), 1.99 (3H, s), 1.98 (3H, s), 1.90-1.99 (2H, m), 1.52 (9H, s), 1.20-1.37 (24H, m), 0.88 (3H, t, J=7.0 Hz). ¹³C NMR (150 MHz, CDCl₃): 170.58, 170.28, 170.11, 170.01, 166.11, 164.97, 155.11, 133.34, 132.90, 129.92, 129.70, 129.56, 129.37, 129.52, 128.22, 97.35, 80.28, 73.88, 71.81, 67.84, 67.70, 67.60, 66.97, 66.50, 61.67, 49.94, 31.83, 29.60, 29.58, 29.56, 29.51, 29.43, 29.27, 29.20, 28.27, 28.13, 25.63, 22.60, 20.60, 20.58, 20.52, 20.49, 14.05. HRMS (ESI-TOF) for C₅₁H₇₃NO₁₆Na⁺ [M+Na]⁺ calcd 978.4821, found 978.4814.

General procedure of synthesis of fatty acid chain analogs was as follows:

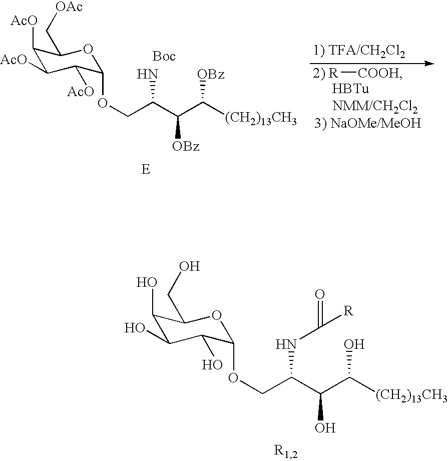

To a stirred solution of E (240 mg, 0.25 mmol) in CH₂Cl₂ (2.4 mL) was added TFA (2.4 mL) at 0° C. and stirred for 2 h at ambient temperature. The solution was evaporated to dryness and co-evaporated with toluene 3 times to give deprotected amine. This compound was dissolved in CH₂Cl₂ and used for the next reaction without further purification.

To the deprotected amine (0.021 mmol) in CH₂Cl₂ (1.0 mL) was added R—COOH (0.031 mmol), HBTu (12 mg, 0.031 mmol) and NMM (31 mg, 0.3 mmol) and stirred at room temperature overnight. The solution was purified by flash column chromatography on silica gel (Hex.: AcOEt 2:1) to give the coupled product as a amorphic solids.

These compounds were dissolved in MeOH (2.0 mL) and 0.5 mol/L NaOMe in MeOH (4 drops) was added. The solution was stirred overnight at room temperature and evaporated to dryness. The residues were purified by flash column chromatography on silica gel (CHCl₃: MeOH 10:1) to give R_{1,2}.

Compound R_{1,2} were synthesized in a manner similar to that described above.

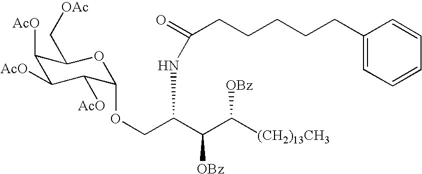

Intermediate of R₁: Yield 28 mg (65%). ¹H NMR (600 MHz, CDCl₃) □: 8.00 (2H, dd, J=1.2 Hz, 8.2 Hz), 7.91 (2H, dd, J=1.2 Hz, 8.3 Hz), 7.61 (1H, t, J=7.4 Hz), 7.53 (1H, t, J=7.4 Hz), 7.47 (2H, t, J=7.8 Hz), 7.38 (2H, t, J=7.8 Hz), 7.25-7.28 (2H, m), 7.15-7.20 (3H, m), 6.57 (1H, d, J=9.7 Hz), 5.69 (1H, dd, J=2.4 Hz, 9.9 Hz), 5.43 (1H, d, J=3.3 Hz), 5.33 (1H, dd, J=3.4 Hz, 10.9 Hz), 5.29-5.32 (1H, m), 5.15 (1H, dd, J=3.6 Hz, 10.9 Hz), 4.81 (1H, d, J=3.6 Hz), 4.62 (1H, tt, J=2.5 Hz, 9.9 Hz), 4.11 (1H, dd, J=6.6 Hz, 13.3 Hz), 4.05 (1H, dd, J=6.0 Hz, 11.0 Hz), 3.96 (1H, dd, J=7.0 Hz, 11.3 Hz), 3.73 (1H, dd, J=2.8 Hz, 10.9 Hz), 3.49 (1H, dd, J=2.4 Hz, 10.9 Hz), 2.64 (2H, t, J=7.8 Hz), 2.35 (2H, t, J=7.7 Hz), 2.10 (3H, s), 1.994 (3H, s), 1.986 (3H, s), 1.94 (3H, s), 1.89-1.93 (2H, m), 1.66-1.78 (4H, m), 1.42-1.48 (2H, m), 1.18-1.35 (24H, m), 0.87 (3H, t, J=7.1 Hz). ¹³C NMR (150 MHz, CDCl₃) □: 172.90, 170.59, 170.39, 170.21, 170.15, 166.50, 165.03, 142.53, 133.49, 133.07, 129.87, 129.77, 129.62, 129.30, 128.62, 128.38, 128.32, 128.24, 125.62, 97.29, 74.14, 71.45, 67.90, 67.53, 67.32, 67.10, 66.68, 61.74, 48.18, 36.65, 35.76, 31.90, 31.22, 29.65, 29.63, 29.60, 29.56, 29.53, 29.34, 29.32, 28.99, 27.86, 25.69, 25.57, 22.67, 20.67, 20.62, 20.58, 20.50, 14.11. HRMS (ESI-TOF) for C₃₆H₆₄NO₉⁺ [M+H]⁺ calcd 1030.5522, found 1030.5507.

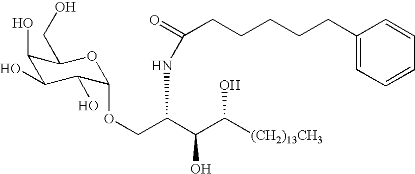

R₁: Yield 14 mg (79%). ¹H NMR (500 MHz, CDCl₃-MeOH 4:1) □: 7.25-7.29 (2H, m), 7.15-7.19 (3H, m), 4.90 (1H, d, J=3.9 Hz), 4.17-4.21 (1H, m), 3.94 (1H, d, J=3.2 Hz), 3.87 (1H, d, J=4.7 Hz), 3.67-3.81 (6H, m), 3.51-3.56 (2H, m), 2.61 (2H, t, J=7.8 Hz), 2.20 (2H, t, J=7.6 Hz), 1.44-1.70 (6H, m), 1.21-1.41 (26H, m), 0.88 (3H, t, J=7.0 Hz). ¹³C NMR (125 MHz, CDCl₃-MeOH 4:1) □: 174.08, 142.25, 128.10, 128.01, 125.42, 99.49, 74.64, 71.86, 70.49, 70.04, 69.53, 68.70, 67.27, 61.69, 50.17, 36.14, 35.46, 32.49, 31.67, 30.93, 29.54, 29.49, 29.46, 29.40, 29.11, 29.00, 28.67, 25.60, 25.40, 22.42, 13.76. HRMS (ESI-TOF) for C₃₆H₆₄NO₉⁺ [M+H]⁺ calcd 654.4575, found 654.4568.

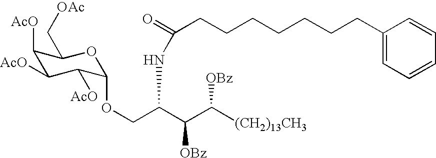

Intermediate of R₂: Yield 28 mg (65%). ¹H NMR (600 MHz, CDCl₃) δ: 7.99 (2H, d, J=7.7 Hz), 7.91 (2H, d, J=7.9 Hz), 7.61 (1H, t, J=7.4 Hz), 7.53 (1H, t, J=7.4 Hz), 7.47 (2H, t, J=7.7 Hz), 7.37 (2H, t, J=7.7 Hz), 7.24-7.28 (2H, m), 7.14-7.18 (3H, m), 6.62 (1H, d, J=9.8 Hz), 5.71 (1H, dd, J=2.3 Hz, 9.9 Hz), 5.43 (1H, d, J=3.2 Hz), 5.35 (1H, dd, J=3.3 Hz, 10.9 Hz), 5.29-5.31 (1H, m), 5.15 (1H, dd, J=3.6 Hz, 10.9 Hz), 4.81 (1H, d, J=3.6 Hz), 4.59-4.64 (1H, m), 4.09-4.12 (1H, m), 4.06 (1H, dd, J=5.9 Hz, 11.2 Hz), 3.97 (1H, dd, J=7.0 Hz, 11.3 Hz), 3.73 (1H, dd, J=2.7 Hz, 10.8 Hz), 3.48 (1H, dd, J=2.2 Hz, 10.9 Hz), 2.60 (2H, t, J=7.8 Hz), 2.35 (2H, t, J=7.7 Hz), 2.10 (3H, s), 2.005 (3H, s), 1.996 (3H, s), 1.94 (3H, s), 1.89-1.93 (2H, m), 1.59-1.76 (4H, m), 1.19-1.43 (30H, m), 0.87 (3H, t, J=7.0 Hz). ¹³C NMR (150 MHz, CDCl₃) □: 172.99, 170.60, 170.38, 170.23, 170.16, 166.52, 165.03, 142.80, 133.47, 133.07, 129.87, 129.76, 129.61, 129.30, 128.61, 128.36, 128.31, 128.18, 125.52, 97.24, 74.16, 71.35, 67.93, 67.52, 67.29, 67.12, 66.70, 61.77, 48.15, 36.71, 35.92, 31.89, 31.47, 29.66, 29.63, 29.60, 29.56, 29.53, 29.34, 29.30, 29.26, 29.23, 29.19, 27.80, 25.72, 25.67, 22.66, 20.67, 20.63, 20.58, 20.49, 14.11. HRMS (ESI-TOF) for C₆₀H₈₄NO₁₅⁺ [M+H]⁺ calcd 1058.5835, found 1058.5819.

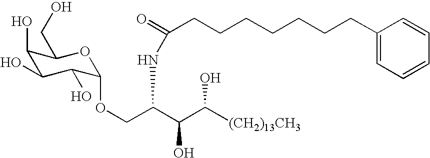

R₂: Yield 14 mg (79%). ¹H NMR (500 MHz, CDCl₃-MeOH 4:1) δ: 7.25-7.29 (2H, m), 7.15-7.18 (3H, m), 4.91 (1H, d, J=3.8 Hz), 4.17-4.22 (1H, m), 3.94 (1H, d, J=3.2 Hz), 3.87 (1H, d, J=4.7 Hz), 3.67-3.81 (6H, m), 3.51-3.56 (2H, m), 2.60 (2H, t, J=7.7 Hz), 2.19 (2H, t, J=7.7 Hz), 1.49-1.70 (6H, m), 1.20-1.41 (30H, m), 0.88 (3H, t, J=7.0 Hz). ¹³C NMR (125 MHz, CDCl₃-MeOH 4:1) □: 174.18, 142.54, 128.11, 127.97, 125.34, 99.49, 74.64, 71.86, 70.48, 70.05, 69.54, 68.71, 67.28, 61.71, 50.17, 36.28, 36.23, 35.67, 32.47, 31.67, 31.24, 29.54, 29.49, 29.47, 29.41, 29.11, 29.04, 29.01, 28.92, 25.60, 25.57, 22.42, 13.76. HRMS (ESI-TOF) for C₃₈H₆₈NO₉⁺ [M+H]⁺ calcd 682.4888, found 682.4880.

In general, the phytosphingosine skeleton was constructed by modification of a method described by Savage and co-workers [R. D. Goff, et al. J. Am. Chem. Soc., 2004, 126, 13602-13603] Garner's aldehyde A was coupled with a Wittig reagent prepared from phosphonium bromide B according to Berova's method [O. Shirota, et al., Tetrahedron, 1999, 55, 13643-13658] to give cis olefin B in 66% yield. Treatment of olefin B with osmium tetroxide gave a corresponding diol C and its undesired isomer. The two hydroxyl groups of diol C were protected with benzoyl groups, and then the isopropylidene group was removed by the successive treatment of TFA, followed by Boc anhydride protection to afford phytosphingosine acceptor D in 67% yield over 3 steps.

Glycosylation of phytosphingosine acceptor D and donor d in the presence of BF₃.OEt₂ gave a predominantly α-configured product. Hydrogenation was avoided as the final deprotection step to ensure accessibility to a more diverse set of compounds. The galactose protecting groups were removed and then protected with acetates to furnish the key intermediate E in 33% over 3 steps.

Compound E was deprotected with TFA to give the deprotected amine. A variety of fatty acyl chain analogs were then couples to the amine to form R after removal of the acetyl groups.

### Example 8

### Recognition of Glycolipids by Murine NKT Cell Lines Results in IL-2 Secretion

### Materials and Methods

### Glycolipids

The compound KRN 7000 was purchased (Kirin, Japan). The remaining compounds were synthesized as described hereinabove.

### 1.2 Hybridoma Assay

CD1d reactive T cell hybridomas with an invariant Vα14i T cell antigen receptor α chain were used, as described (Sidobre, S., et al. (2004) Proc. Natl. Acad. Sci. USA 101, 12254-12259). T cell hybridomas were stimulated with 0.0001-1 μg/ml of the indicated glycolipids added to CD1d transfected A20 B lymphoma cells, as described (Elewaut, D., et al. (2003) J. Exp. Med. 198, 1133-1146). As a measure of T cell activation, IL-2 and IFN-γ release into the tissue culture medium was measured after 16 hours culture by an ELISA assay.

### In Vitro Cytokine Secretion Assay Using Human NKT Cell Lines

IL-2 secretion by the Vα 24i human NKT cell line was determined by ELISA (BD Pharmingen, San Diego, Calif.) after culture for 16 hours. For these assays, 1×10⁵ Vα 24i human NKT cells were co-cultured with 4×10⁵ irradiated, immature CD14⁺ dendritic cells, in the presence of the glycolipid compounds at 10 μg/ml in a 96-well flat-bottom plate [Wu et al. PNAS 2005 102: 1351].

### Results

In order to test whether glycolipids with modifications the lipid moiety of α-GalCer affected the immunogenicity of the compound, a series of glycolipids with varied modifications of this region were synthesized and assayed.

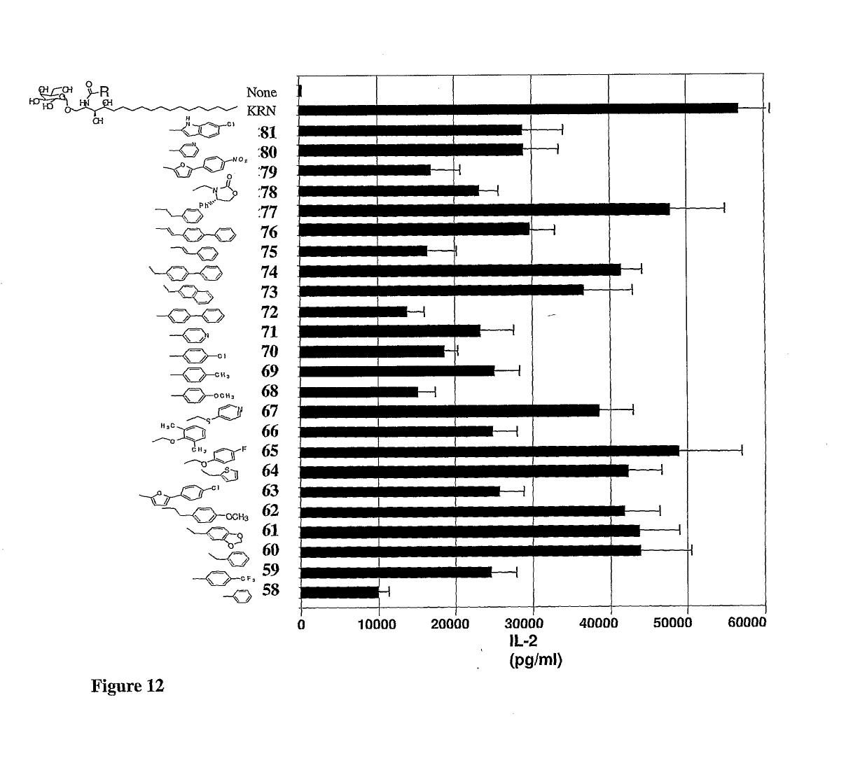

Mouse Vα14i NKT cells immortalized by cell fusion provided a convenient method for assaying the ability of the synthetic glycolipids to activate T cells. As shown in FIG. 12, a number of the compounds (60, 61, 62, 64, 65, 74, 77) stimulated significant IL-2 release from the hybridomas when used at 1 μg/ml, however KRN7000 (α-GalCer) appeared to stimulate the greatest amount of IL-2 release.

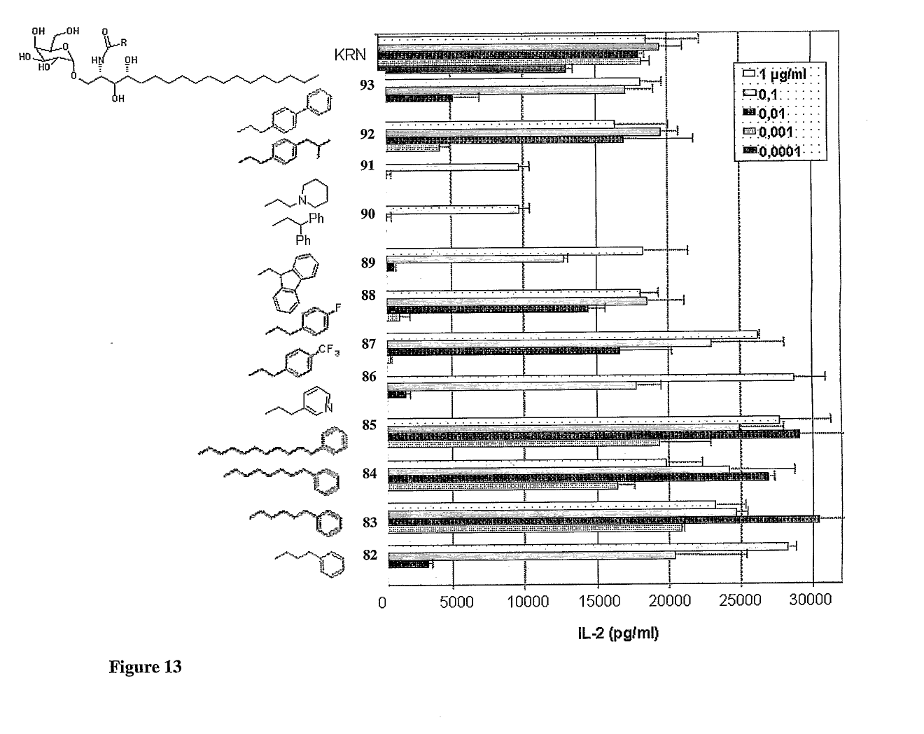

Another series of compounds were evaluated for their ability to stimulate IL-2 release, when provided at various concentrations (FIG. 13). In this case, several of these compounds stimulated greater IL-2 release, as compared to KRN7000 (α-GalCer), in particular, compounds with a terminal phenyl substituent.

The simplest benzoyl analog 58 showed only slight activity. Introduction of either electron donating groups (68; 4-OCH3, 69; 4-CH3) or withdrawing groups (70; 4-Cl, 59; 4-CF3) onto the benzene ring increased their activity. The other benzoyl analogs, 4-Pyridyl 80, 3-pyridyl 71, indole analog 81 and biphenyl analogs 72, 63 and 80 also exhibited similar trends. However, their activities still remained about half of α-GalCer.

Benzyl analogs 60, 61, 69, 73 and 74 showed improved IL-2 production compared with the benzoyl analogs. Of these compounds, smaller aromatic groups such as 60, 61 and 69 showed better activity than that of analogs 73, 74 bearing larger aromatic groups. The activities of thiophene analog 69 and benzene analog 60 were comparable.

Phenylethylene analogs 62, 77, 87 and 88 demonstrated comparable or even more potent IL-2 production compared with the benzyl analogs. The 4-CF3 analog 87 and 4-isobutyl analog 82 possessed slightly better activities compared with the 4-OCH3 analog 62 and 4-F analog 88. Substitution of the phenyl group with the 3-pyridyl group 86 diminished IL-2 production dramatically, which contradicted the trends of benzoyl analogs (58 and 71). In addition, the introduction of a trans-double bond as a spacer group significantly reduced IL-2 production compared with the saturated analog (75 and 77). The biphenyl analog 93 also showed a decreased activity. Introduction of a basic functional group, such as piperidinylethyl analog 91 demonstrated a significant reduction in cytokine production. This result may be because of repulsion between the basic amine moiety and the hydrophobic residue in the binding pocket. The 4-Fluorophenoxymethyl analog 65 gave a similar activity as the corresponding carbon analog 88. On the other hand, the 2,6-dimethyl substituted analog 66 exhibited a reduced activity, suggesting that the binding pocket was not large enough to accept bulky substituents. Similar results were observed in compounds 72, 89 and 90, bearing bulky substituent such as 4-biphenylmethyl, 2,2-diphenylmethyl and 9-fluorenyl, respectively.

Further extension of spacer chain length gave best results under these conditions. The activity of 3-phenylpropyl analog 82 was moderate. However, the 5-phenylpentyl 83, 7-phenylheptyl 84 and 10-phenyloctyl 85 all showed a significant increase of IL-2 production. Compounds 83-85 were much more potent than α-GalCer.

### Example 9

### Recognition of Glycolipids by Human NKT Cell Lines Results in NKT Cell IFN-γ and IL-4 Secretion

### Materials and Methods

### Generation of Vα24i Human NKT Cell Line

Human NKT cell lines, expressing the Vα24i T cell receptor as well as CD161, were generated as follows: Anti-CD161 monoclonal antibodies, and anti-CD14 monoclonal antibodies, each coupled to magnetic beads (Miltenyi biotec, Auburn, Calif.), were used sequentially to isolate CD161⁺ cells and CD14⁺ cells from leukopaks. Immature dendritic cells were generated from the CD14⁺ cells after a two-day incubation in the presence of 300 U/ml GM-CSF (R&D systems, Minneapolis, Minn.) and 100 U/ml IL-4 (R&D systems, Minneapolis, Minn.). Following irradiation with 2000 rads, the immature dendritic cells were co-cultured with syngeneic CD161⁺ cells in the presence of 100-0.1 ng/ml of alpha-galactosylceramide and 10 IU/ml of IL-2 (Invitrogen, Carlsbad Calif.) for 10 to 14 days. After stimulating the CD161⁺ cells a second time with alpha-galactosylceramide-pulsed, irradiated immature dendritic cells, NKT cell lines were shown by flow cytometry to express both CD161⁺ and V 24i TCR (99% purity).

In some cases, Hela cells were transfected with a human CD1d construct [Xing et al. 2005. Bioorg Med Chem 13: 2907], and were used to present the glycolipids, via pulsing with the respective compounds at the indicated concentration, to NKT lines.

IFN-γ secretion by the Vα 24i human NKT cell line was determined by ELISA (BD Pharmingen, San Diego, Calif.) after culture for 16 hours. For these assays, 1×10⁵ Vα 24i human NKT cells were co-cultured with 4×10⁵ irradiated, immature CD14⁺ dendritic cells, in the presence of the glycolipid compounds at 10 μg/ml in a 96-well flat-bottom plate.

### Results

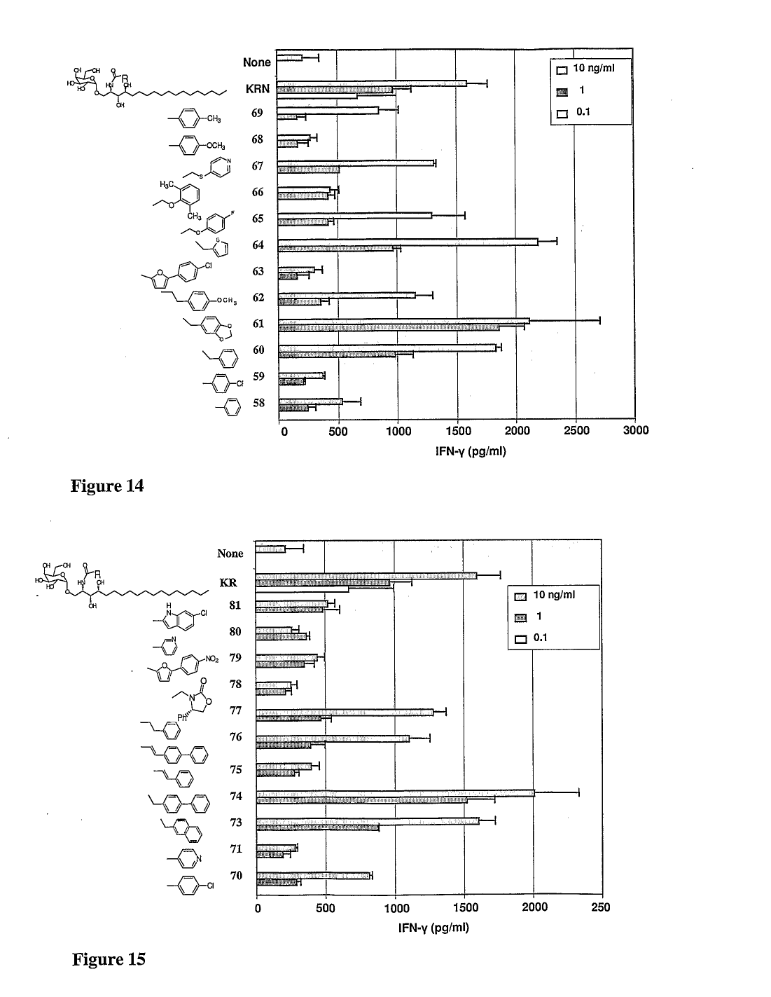

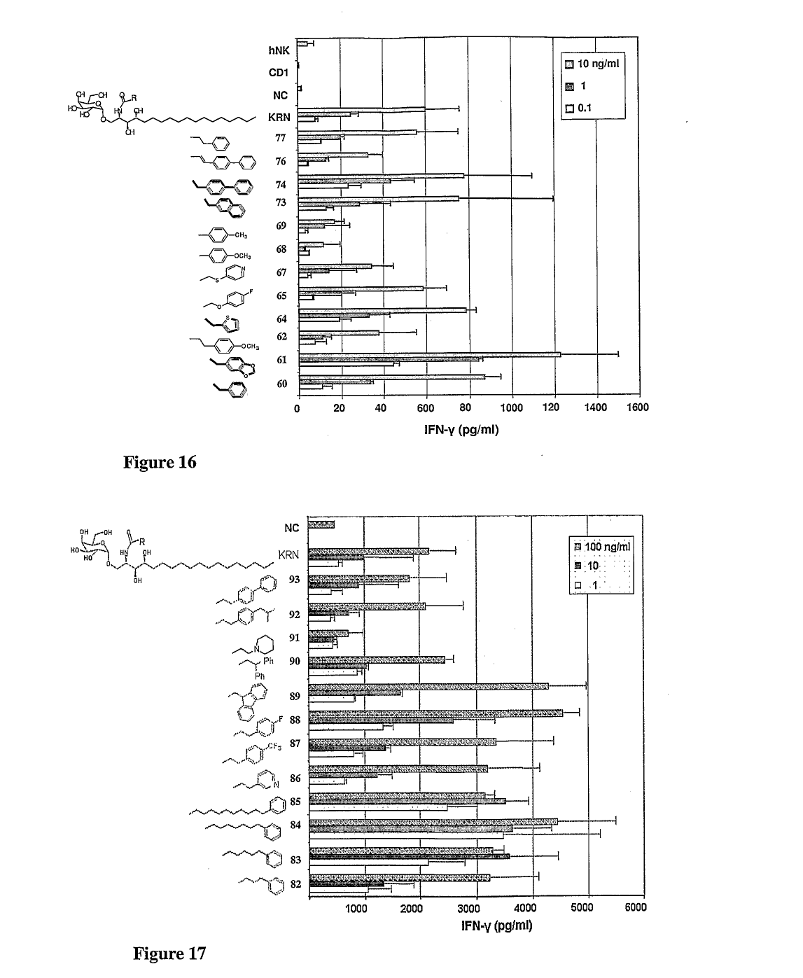

IFN-γ secretion from a Vα24i NKT cell line were assessed, after stimulation with irradiated, syngeneic CD14⁺ immature dendritic cells in the presence of 10, 1 and 0.1 μg/ml of the respective glycolipids and 10 IU/ml of IL-2 (FIGS. 14, 15 and 16).

Stimulation of the NKT cell line by many of the glycolipid compounds resulted in significant IFN-γ secretion, when compared to the negative control, with some specific compounds providing greater greater IFN-γ secretion as compared to α-GalCer.

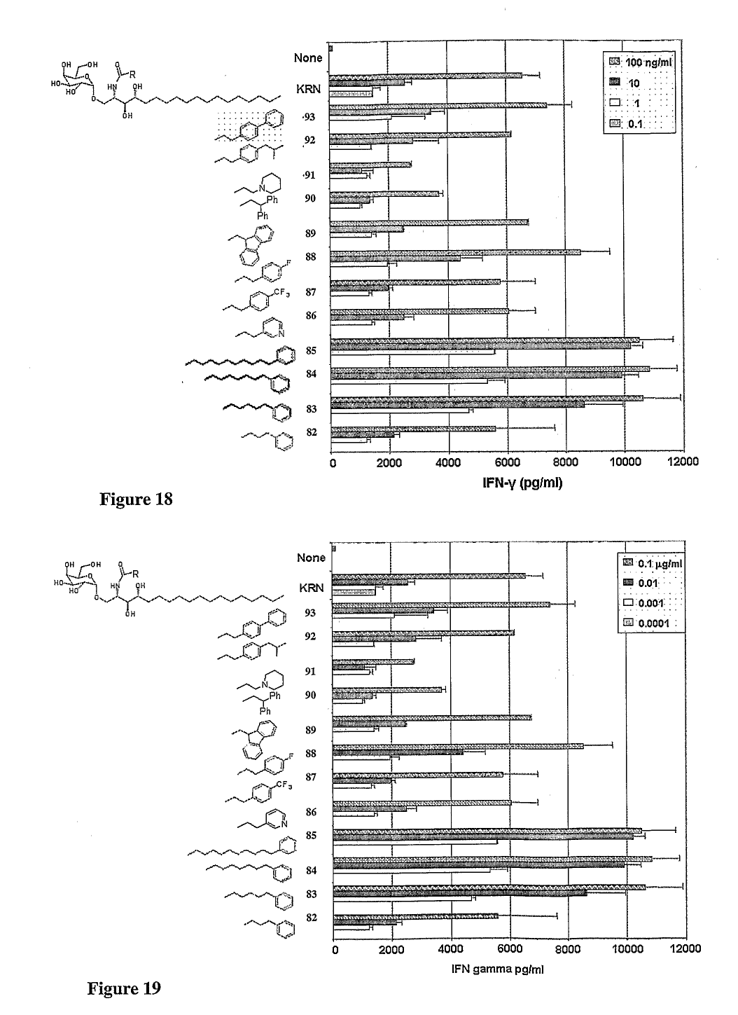

As illustrated in FIGS. 17 and 18, additional glycolipid compounds were prepared and evaluated for interferon-γ secretion by human NKT cells in response to glycolipid presentation by CD14⁺ DCs, as compared to α-GalCer, at a concentration of 100-0.1 ng/mL. Compounds 83, 84 and 85 in these figures consistently stimulating greater IFN-γ secretion, at all doses evaluated, as compared to KRN, and other compounds.

Hela cells expressing human CD1d were also effective in presenting the glycolipids to human NKT cells, with similar profiles in terms of stimulating NKT cell IFN-γ secretion (FIG. 19).

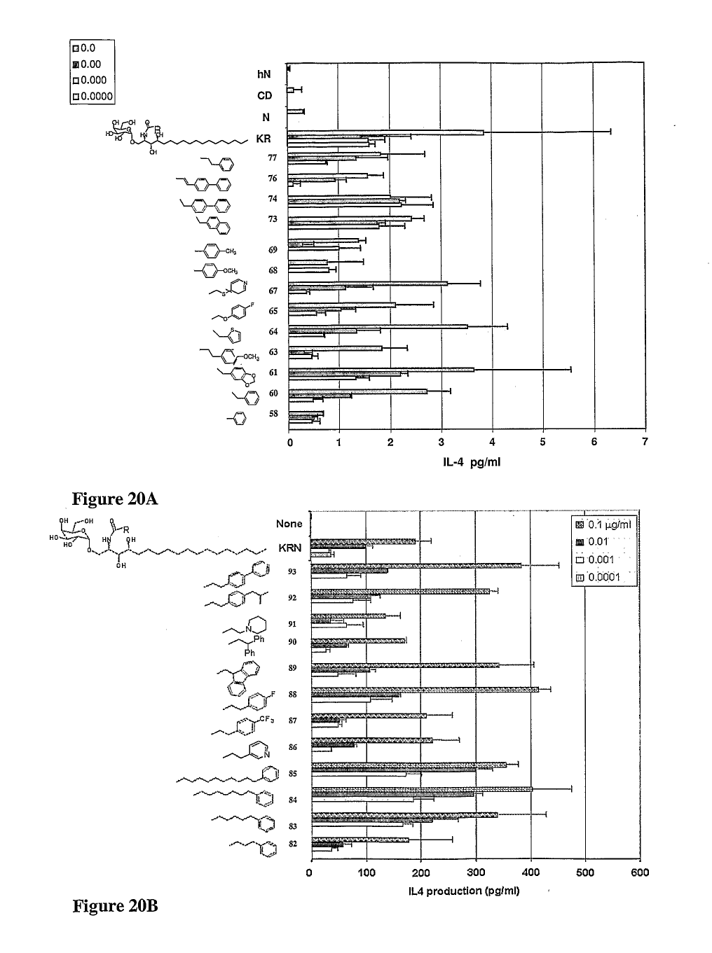

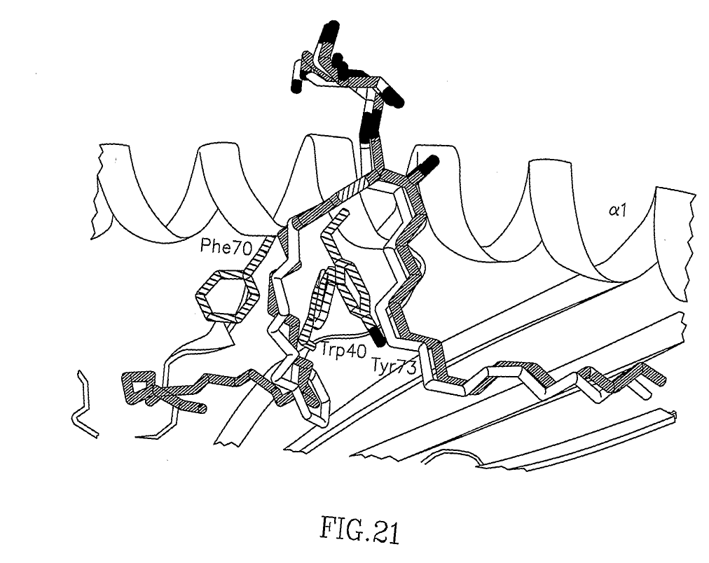

Compounds effective in stimulating IFN-γ secretion were also found to stimulate IL-4 secretion (FIG. 20).

The longer alkyl chain analogs 83-85 were more potent toward both IFN-γ and IL-4 production than the shorter alkyl chain analogs. The 7-phenylheptyl analog 83 exhibited a high ratio of IFN-γ/IL-4 activity and was the best among these compounds. However, compounds 73 and 77 are more selective for IL-4 production while 82 is specific for IFN-γ production.

### Example 10

### Possible Structural Basis for Glycolipid Recognition

Recent glycolipid-CD1d protein crystal structures revealed the existence of various aromatic residues, Tyr73, Phe114, Phe70 and Trp114, which might be able to interact with the phenyl group of the present compounds comprising fatty acyl chain analogs. According to these crystal structures, the benzoyl analogs 8-14, which have no spacer chain, seemed to be too short to interact with these aromatic residues. To further investigate the interactions between the phenyl analogs and human CD1d, Autodock 3.0 [G. M. Morris, et al., J. Comp. Chem., 1998, 19, 1639-1662] was utilized to model the binding of these compounds in the hCD1d hydrophobic groove (FIG. 21). Compounds 40-42 were individually docked and their results did not vary significantly from the crystal structure of α-GalCer bound to hCD1d [M. Koch, et al., Nat. Immunol., 2005, 6,819-826]. In each case, the phytosphingosine tail extended into the F′ pocket and the A′ pocket was occupied by the fatty acyl chain with the galactose headgroup presented in nearly all the same configuration. However, introduction of a terminal phenyl group in the α-GalCer analogs seemed to promote additional specific interactions between compounds 40, 41 and the phenol ring of Tyr73 and between 42 and Trp40.

Biphenyl analogs 16-18 and cinnamoyl analogs 30, 32, lacked a flexible fatty acyl chain and may not have been able to extend into the A′ pocket deep enough to make any specific interactions. Extension of the spacer chain length, such as the benzyl analogs 19-22, the phenylethylene analogs 24-28, and the 4-fluorophenoxymethyl analog 34, allowed for tighter binding via π-π interaction, possibly with the aromatic side-chain of Tyr73. Further extension of the spacer chain length, such as pentamethylene analog 83, heptamethylene analog 84 and decamethylene analog 85, were more suitable for tighter binding. These results suggest that the introduction of π-π interaction potentiates IL-2 production, probably through the formation of a tighter ligand-CD1d protein complex.

These fatty acyl chain analogs bearing aromatic groups seemed to possess more potent activity than the corresponding simple fatty acyl chain analogs. Compounds 83-85 bearing 5, 7, 10 carbons spacer chain, respectively, demonstrated much more potent IL-2 production than that of other groups, with α-GalCer bearing a C26 fatty acyl chain. These results suggest that introduction of a terminal aromatic group on the fatty acyl chain causes an enhancement of the activity through interactions between the aromatic residues in the hydrophobic pocket of CD1d protein and the lipid tail.

### Examples 11-16

### Materials and Methods

Mice

6-8 week-old Balb/c female mice were purchased from Taconic. Balb/c transgenic mice expressing a TCR specific for the tumor antigen P1A35-43:Ld complex have been described [Sarma, S. et al. J. Exp. Med. 189, 811-820 (1999)]. Jα18^{-/-} mice on Balb/c background were obtained from Dr. Moriya Tsuji (New York University School of Medicine, New York, N.Y.). Mice were maintained under specific pathogen free conditions. All experiments were conducted according to institutional guidelines.

Cell Lines:

The plasmacytoma J558 cell line, and the MHC class I mutant cell line J558Ld⁻ were used [Guilloux, Y., et al. Cancer Res. 61, 1107-12 (2001)]. The Meth A fibrosarcoma was provided by Dr. Zihai Li (University of Connecticut Health Center, Farmington, Conn.). Cell lines were cultured in RPMI 1640 medium supplemented with 10% FCS, 100 μg/ml penicillin/streptomycin and 2 mM glutamine. All lines tested negative for Mycoplasma by Hoechst staining and PCR reaction (ATCC).

Reagents:

Rat mabs for MHC class II (TIB120, M5/114.15.2), granulocytes (RB6-8C5, Gr-1), B220 (TIB146, RA3-3A1), F4/80 (HB198), CD4 (GK 1.5) and CD8 (TIB211, 3.155) were from the ATCC (Rockville, Md.). Anti-CD16/32, PE-conjugated anti-CD8α, CD11b, B220, CD4, Vα8.3, IFN-γ, IL-2, H-2L^{d}/H-2 D^{b}, CD62L, CD69, B7-H1, B7-DC, and APC CD11c were from BD PharMingen (San Diego, Calif.) or eBioscience (San Diego, Calif.). Sheep anti-rat IgG conjugated to magnetic beads were from Dynal (Lake Success, N.Y.). Anti-CD11c and CD8 Microbeads® were from Miltenyi Biotec (Bergisch Gladbach, Ger). The other reagents were RPMI 1640 (GIBCO, Grand Island, N.J.), FCS (GIBCO), carboxyfluorescein diacetate, succinimidyl ester (CFSE; Molecular Probes, Eugene, Oreg.), ACK buffer (BioSource: Grass Valley, Calif.), 30% BSA solution (Sigma, St. Louis, Mo.). α-GalCer(2S,3S,4R-1-O(α-galactopyranosyl)-2(N-hexacosanoylamino)-1,3,4-octadecanetriol) was provided by the Pharmaceutical Research Laboratory, Kirin Brewery (Gunma, Japan) and diluted in PBS.

Induction of Cell Death:

Tumor cells were harvested, washed twice with RPMI 1640, resuspended to 10×10⁶/ml in RPMI and irradiated with 75 Gy. Detection of apoptotic tumor cells used the Annexin V-FITC Apoptosis Detection Kit (PharMingen, San Diego, Calif.), after which flow cytometry (FACS Vantage SE, Becton Dickinson) was performed. Within 24 hrs, 24% of the tumor cells were apoptotic, i.e., annexin V positive but PI negative (as described further hereinbelow). By 48 h, 57% of the irradiated cells underwent secondary necrosis; and 72 h later, 84% of them were necrotic (PI⁺). Therefore we refer to the irradiated cells that we injected as “dying cells”.

Cell Preparation:

CD8⁺ P1A-specific, T cells were prepared from meshed cell suspensions of TCR transgenic lymph nodes and spleen by depleting B220, CD4, F4/80 and MHC class II-expressing cells using sheep anti-rat IgG Dynabeads®. For CFSE labeling (Molecular Probes), the cells at 10⁷/ml in PBS were incubated with 5 μM CFSE for 10 min at 37° C. The reaction was stopped by washing three times with PBS.

In Vivo Delivery of Dying Tumor Cells, Dc Maturation Stimuli, & Tumor Protection Assay:

2×10⁷ irradiated J558-Ld⁻ cells, were injected i.v. or s.c. into Balb/c mice with or without α-Gal Cer as a DC maturation stimulus. In some experiments we compared α-Gal Cer to agonistic anti-CD40 mAb (1C10, 25 μg i.p.) or the toll like receptor ligands, poly IC (50 μm, Invivogen) or lipopolysaccharide (20 μgm, Sigma). The mice had been given CFSE-labeled PICTL CD8⁺ T cells i.v. 1 d earlier, or were naive animals. In some experiments, mice were sacrificed 3 d later, and T cell division and activation in spleen were analyzed by flow cytometry. Additionally, 7 d or 2 months later, 5×10⁶ live J558 tumor cells were inoculated subcutaneously. 5×10⁶ Meth A fibrosarcoma was used as a control tumor for challenge. Tumor cell growth was measured with calipers every other day. Mice were scored positive for tumor as soon as tumors became palpable and grew progressively. Mice were euthanized when tumor size exceeded 400 mm².

Mice were also injected i.v. with 2×10⁷ dying, irradiated, CFSE-labeled A20 tumor cells, and after 2 hours, spleens were processed, sectioned, probed with anti-mouse CD11c-PE and evaluated by immunofluorescence microscopy, for uptake of dying A20 tumor cells by CD11c+ splenic DCs.

Mice were also injected i.v. with PBS, 2 μg α-Gal Cer, 5×10⁶ irradiated A20 cells with or without 2 μg α-Gal Cer i.v. Two weeks later, mice were challenged with a lethal tumorogenic dose of 5×10⁶ live A20 s.c. The mice were monitored every other day for tumor growth and were scored positive when the tumors were palpable.

Flow Cytometry:

T-cell division, phagocytosis of CFSE-labeled tumor cells and acquisition of cell surface-activation markers were determined by flow cytometry. Briefly, spleens were harvested and low-density splenocytes were stained with CD11c-APC and CD8α-PE, CD11b-PE, B220-PE or CD4-PE for the up-take examination. For in vivo proliferation of P1CTL T-cells, spleens were harvested and the splenocytes suspension was staining with CD8α-Cy, Vα8α-PE, CD25-PE, CD62L-PE. For intracellular cytokine staining, splenocytes were stimulated in vitro with 1 □g/ml P1A peptide in 5 μg/ml brefeldin A (Sigma-Aldrich) at 37° C. for 4 hrs. The cells were first stained with CD8α-Cy-Chrome™, fixed and permeabilized with cytofix-cytoperm buffer (BD Biosciences), and stained with PE conjugated mAbs to IL-2 or IFN-γ.

### In Vivo Depletion of CD4⁺ and CD8⁺ T Lymphocytes:

Mice vaccinated with J558 and α-Gal Cer were injected with ascites containing 1 mg of rat monoclonal anti-CD4 (clone GK 1.5) or anti-CD8 (clone 53-6.72). The mice received three daily injections, the first one i.v. one day before the challenge, and two i.p. injections on the day of the challenge and one day after the challenge. Control mice received 1 mg of Rat IgG (Jackson Laboratories). The depletion was monitored by staining with anti-CD4 and anti-CD8 antibodies followed by flow cytometry (BD PharMingen).

### Example 11

### Intravenously Administered Dying Tumor Cells are Selectively Captured by DCs

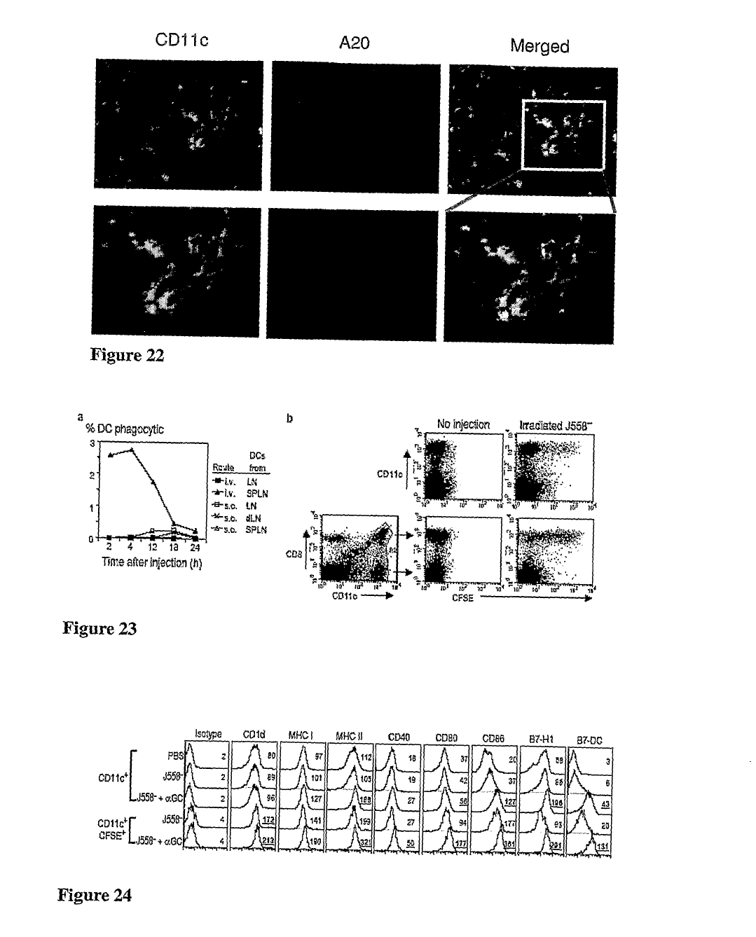

In order to determine whether DCs could take up tumor cells, mouse spleen cells were assessed for their ability to take up A20 lymphoma cells. Mice injected intravenously with dying, irradiated, CFSE-labeled A20 tumor cells showed selective uptake of the dying A20 tumor cells by CD11c+ splenic DCs (FIG. 22).

MHC class I negative, J558Ld⁻ (J558⁻) variant of a mouse plasmacytoma were similarly evaluated. This variant has lost expression of cell surface MHC class I and multiple antigen presentation genes, including TAP-1, TAP-2, LMP-2, and LMP-7, due to malfunction of the proto-oncogene pml. Because the tumor cells lack MHC class I, and also fail to express MHC class II (data not shown), recipient antigen presenting cells would have to capture and process J558⁻ cells to elicit T cell responses.

To verify that J558⁻ underwent cell death following 75 Gy γ-irradiation, cells in culture, as a function of time, were stained with Annexin V and propidium iodide (data not shown). Uptake of CFSE-labeled, irradiated, “dying” J558⁻ cells in vivo by lymph node and splenic DCs following injection by i.v. and s.c. routes was evaluated by flow cytometry.

FIG. 22 demonstrates phagocytosis of i.v. injected tumor cells by CD11c⁺ splenic DCs, within 2 hours post-injection. In contrast, few if any CFSE-labeled cells were detected in splenic or lymph node DCs when the tumor cells were injected by the s.c. route (FIG. 22b). CFSE-labeled tumor material was primarily detected in the CD11c⁺ DC-enriched populations and only the CD8α⁺ CD11c⁺ DC subset endocytosed the injected CFSE-labeled tumor.

Few CFSE-labeled cells were taken up by individual CD11c⁻ fractions of spleen, marked for CD11b, B220 or CD4 (data not shown). A single injection of 20×10⁶ J558⁻ cells was limiting, since 5×10⁶ resulted in much lower CFSE labeling of DCs. Therefore dying J558 tumor cells were selectively captured by DCs in vivo when administered intravenously.

### Example 12

### α-Gal Cer Injection Leads to Rapid Maturation of Phagocytic DCs In Vivo

α-Gal Cer is a nonmammalian glycolipid that is presented by CD1d molecules to an invariant T cell receptor expressed by innate NKT lymphocytes. A single i.v. dose of α-Gal Cer activates NKT cells, and this leads to full DC maturation in vivo, defined as the ability to initiate combined CD4⁺ and CD8⁺ T cell immunity.

In order to determine the maturation status of DCs that phagocytosed dying tumor cells, mice were injected with CFSE-labeled, irradiated, MHC class I negative, J558⁻ cells in the presence or absence of α-Gal Cer. Five hours post-injection DCs were analyzed by flow cytometry for the expression of a number of cell surface molecules that change during DC maturation. Injection of tumor cells alone had little effect on the phenotype of the total CD11c⁺ splenic population relative to PBS controls, however, injection of α-Gal Cer (not shown) or co-injection of tumor with α-Gal Cer resulted in the maturation of the total CD11c⁺ DC population, as indicated by up-regulation of MHC II, CD80, CD86, B7-H1, and B7-DC 5 hours later (FIG. 23).

DCs that had captured dying tumor cells (i.e., cells positive for CD11c and CFSE), represented <3% of the splenic DCs (FIG. 22a), had higher levels of CD1d and other markers (FIG. 23), and strongly upregulated the expression of antigen presenting and costimulatory molecules in response to α-Gal Cer administration. Thus, the administration of α-Gal Cer resulted in DCs that captured tumor cells to exhibit numerous changes typical of maturation.

### Example 13

### Intravenous Administration of Dying Tumor Cells and α-Gal Cer Induces Tumor Immunity

To determine if immunity was induced by delivery of dying tumor cells to DCs, we used a tumor protection assay. Naïve Balb/c mice were injected with PBS, α-Gal Cer, 20×10⁶ irradiated J558⁻ tumor cells alone, or in combination with α-Gal Cer, or 5×10⁶ irradiated A20 cells and 2 μg α-Gal Cer i.v. One to three days following vaccination, mice were challenged with 5×10⁶ J558 live tumor cells s.c, for plasmacytoma cells, or two weeks post vaccination with A20, mice were challenged with a lethal tumorogenic dose of 5×10⁶ A20 cells s.c.

Plasmacytomas grew progressively within 7-10 days in mice that had received either PBS or α-Gal Cer alone (FIG. 24).

Injection with irradiated J558⁻ tumor cells alone protected 15% of the mice from a subsequent J558 challenge (3 of 20 mice in 4 experiments), and 88% of the mice (22 of 25 in 5 experiments) were protected and remained tumor-free following vaccination with combined irradiated J558⁻ cells and α-Gal Cer (FIG. 24A). Similarly, PBS or α-Gal Cer pretreatment alone resulted in the development of palpable tumors, whose diameters increase in size, as a function of time, however mice were protected and remained tumor-free following vaccination with combined irradiated A20 cells and α-Gal Cer (FIG. 24B).

In order to evaluate the specificity of the antitumor immune response, mice were challenged with 5×10⁶ live Meth A sarcoma cells s.c. after vaccination with irradiated J558 with α-Gal Cer. Vaccination with dying J558⁻ and α-Gal Cer protected mice against a challenge of J558 but not Meth A cells (FIG. 24C).

In order to compare α-Gal Cer with other DC maturation stimuli, use of an agonistic anti-CD40 monoclonal antibody, and TLR stimuli, LPS and poly IC were tested in this context. The glycolipid was more efficient at inducing protective immunity than the other compounds tested (FIG. 24D). In order to assess immunological memory, vaccinated mice were evaluated for their protection against J558 challenge 2 months (FIG. 24E) and 4 months (not shown) after vaccination. Vaccination with J558 and α-Gal Cer was effective only when tumor cells were injected by the i.v. and not the s.c. route (FIG. 24F), a fact which correlated with the finding in Example 1 that intravenous injection of tumor cells was necessary for DC internalization. Tumor resistance was found when animals were vaccinated even 3 days after the injection of the tumor cells (FIG. 24G), with the therapeutic response manifest when the tumor dose was 1×10⁶ but not 5×10⁶ cells. Protection was demonstrated in using either dose. Thus long-lived tumor immunity can be elicited by a single vaccination with irradiated J558 and α-Gal Cer, which also has a therapeutic effect.

### Example 14

### Combined Innate and Adaptive Resistance Induced by Dying Tumor Cells and α-Gal Cer

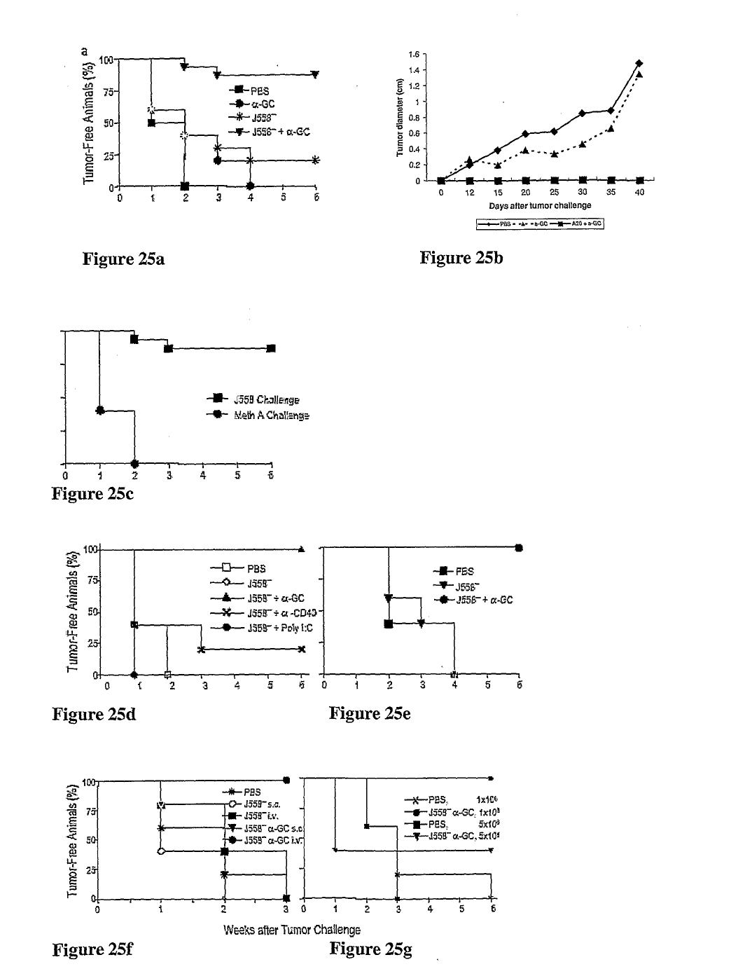

In order to identify resistance mechanisms involved in the tumor immunity engendered in Example 13, mice vaccinated with dying MHC class I⁻ J558 cells and α-Gal Cer were challenged with live MHC class I negative or positive J558 tumor cells. The vaccinated mice were protected against the MHC class I⁺ J558 cells but not to MHC class F J558 tumor cells (FIG. 25), suggesting that CD8⁺ T cells were required for resistance. NKT cells as expected were also required for effective vaccination since Jα281^{-/-} mice (also termed Jαb 18^{-/-}), which cannot respond to α-Gal Cer because they lack NKT cells 39, failed to develop immunity to dying cells plus glycolipid (FIG. 25b). To further evaluate the type of adaptive T cells required for protective immunity 8 weeks after a single vaccination, the immune mice were injected with depleting antibodies specific for CD4, CD8, or control IgG, and then challenged with J558 cells 1 day later. We verified by FACS that anti-CD4 and anti-CD8 antibodies depleted the respective cell populations within 2 days, and that the mice remained depleted of these T cells for 2 weeks, when they began to repopulate slowly. As shown in FIG. 25c, mice injected with control IgG remained resistant to J558 challenge. However, depletion of either CD4⁺ or CD8⁺ T cells from vaccinated mice significantly abrogated tumor immunity elicited by J558 with α-Gal Cer. Therefore, both innate NKT cells and adaptive CD4⁺ and CD8⁺ T cells contribute to the tumor resistance induced by DCs capturing dying cells in vivo.

### Example 15

### Co-Injection of α-Gal Cer and Dying Cells Activates Antigen-Specific CD8⁺ T Cells

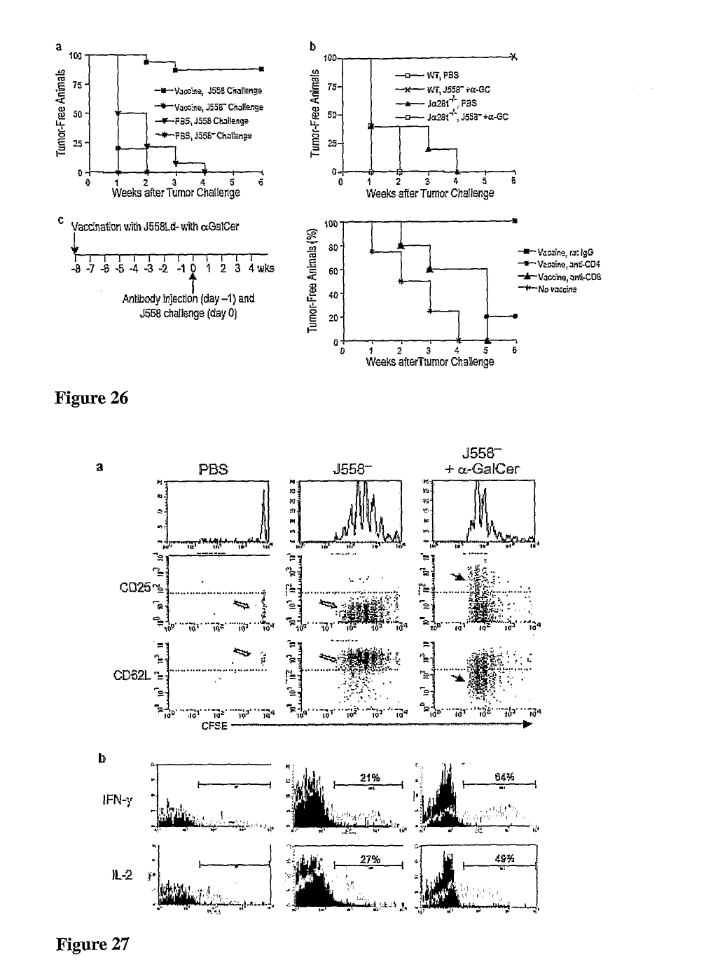

To document the consequences of α-Gal Cer for the quality of the T cell response to the injection of irradiated tumor, the P1CTL mouse, a CD8⁺ TCR transgenic line specific for the P1A tumor antigen presented on L^{d} MHC class I molecules [Sarma, S. et al. J. Exp. Med. 189, 811-820 (1999)] was used. 20×10⁶ irradiated, MHC class I negative, J558⁻ cells were injected alone, or in combination with α-Gal Cer into mice that had received CFSE-labeled P1CTL cells 1 day earlier. T cell proliferation and phenotype were analyzed 3 days later with flow cytometry. In the absence of α-Gal Cer, DCs could cross-present P1A from dying tumor cells to CD8⁺ P1CTL T cells, driving the T cells into multiple cycles of proliferation (FIG. 26, top row). This is consistent with the capacity of CD8⁺ DCs to present antigens on both MHC class I and II products from dying cells in the steady state^{22,23}. However, the proliferating P1CTL T cells retained markers typical of naïve cells, i.e., low CD25 and high CD62L (FIG. 26, white arrows). In contrast, in the mice that had received J558 plus α-Gal Cer, the T cells proliferated more extensively and many began to show an activation phenotype, indicated by the upregulation of CD25 and downregulation of CD62L (FIG. 26, black arrows). Furthermore, T cells that had been stimulated in the presence of α-Gal Cer adjuvant in vivo were able to produce significantly more IFN-γ and IL-2 upon brief restimulation with P1A peptide in vitro (FIG. 26, compare right and middle panels). Taken together the results shown that DCs process antigens from tumor cells and induce the proliferation of antigen-specific T cells in vivo, but a maturation stimulus is required for the differentiation of effector T cells, as well as protective immunity.

### Example 16

### Proof that Mature DCs Present Tumor Antigen and Transfer Tumor Immunity

To verify that mature DCs were responsible for the presentation of antigens from the captured dying tumor cells and also elicited tumor immunity, DCs were isolated from mice injected with dying J558 tumor cells without or with α-Gal Cer. The CD11c⁺ DC-enriched and CD11c⁻ DC depleted cells from spleen were added as stimulators in cultures of naïve CD8⁺ P1CTL TCR transgenic T cells without further antigen. When α-Gal Cer had been co-administered, the isolated DCs were much more effective at stimulating proliferation of naïve CD8⁺ T cells in culture, while CD11c⁻ cells were inactive (FIG. 27a, closed squares in right panel).

CD11c⁺ DCs were isolated from spleens 4 hours after immunization and the DCs were transferred to naïve animals to test their capacity to stimulate proliferation of CFSE-labeled CD8⁺ P1CTL T cells in vivo (FIG. 27b). 3 days later, P1CTL proliferation was detected only in response to DCs from mice given tumor with α-Gal Cer (FIG. 27b, black arrow). CD11c⁻ non-DCs from the same mice were not able to stimulate P1CTL T cells, and DCs from mice that had received J558⁻ without α-Gal Cer failed to stimulate P1CTL above the background. Finally, to test if the antigen presenting mature DCs were critical for inducing protective tumor immunity, we transferred DCs or non-DCs from the vaccinated mice into naïve mice and then challenged them with live J558 tumor cells (FIG. 27c). When naïve mice had been given 1.5×10⁶ CD11c⁺ DCs from donor mice injected with dying J558⁻ together with α-Gal Cer, 58% of mice (10 of 17 mice tested) were fully protected. CD11c⁻ non-DCs and CD11c⁺ DCs from mice injected with PBS or dying J558⁻ alone, failed to transfer protection to naïve mice (0/12). These data provide direct evidence that mature antigen capturing DCs are responsible for presentation of tumor antigen and the adjuvant action of α-Gal Cer in vivo.

### Examples 17

### Materials and Methods

Mice. BALB/C and C57B1/6 mice were from Taconic. Jα18−/− mice, which lack NKT cells, on the C57B1/6 background were a kind gift from M. Tanaguchi (Chiba University, Chiba, Japan). Mice were maintained under specific pathogen-free conditions and used at 7-8 wk of age, following guidelines of our Institutional Animal Care and Use Committee.

Cell lines. The plasmacytoma J558 and the MHC class I mutant cell line J558Ld− have been described previously [Guilloux, 2001 Cancer Res 61(3): 1107-12]. These cell lines were cultured in RPMI 1640 medium supplemented with 10% Fetal Calf Serum (FCS), 100 μg/ml penicillin/streptomycin, and 2 mM glutamine. All lines tested negative for Mycoplasma by Hoechst staining and PCR reaction (American Type Culture Collection).

Reagents. αGalCer (2S,3S,4R-1-O(α-galactopyranosyl)-2(N-hexacosanoylamino)-1,3,4-octadecanetriol) was provided by Kirin Brewery and diluted in PBS. Compound 24 (3-O-sulfo-alpha-galactosylceramide) and compound 27 (sphingosine-truncated (C9)) are analogues of αGalCer, as described herein. Rat mAbs for MHC class II (TIB120, M5/114.15.2), granulocytes (RB6-8C5, Gr-1), B220 (TIB146, RA3-3A1), F4/80 (HB198), CD4 (GK 1.5), and CD8 (TIB211, 3.155) were obtained from the American Type Culture Collection. Anti-CD16/32, PE-conjugated anti-CD8α, I-Ad, CD40, CD80, CD86, CD119, B7-H1/PD-L1, B7-DC/PD-L2 and allophycocyanin-CD11c were obtained from BD Biosciences or eBioscience. Anti-CD11c microbeads were from Miltenyi Biotec (Auburn, Calif.). The other reagents were RPMI 1640 (GIBCO BRL), FCS (GIBCO BRL), ACK buffer (BioSource30% BSA solution; Sigma-Aldrich).

Induction of cell death. Tumor cells were harvested, washed twice with RPMI 1640, resuspended to 1×10⁷/ml in RPMI 1640, and irradiated with 75 Gy. To detect apoptotic tumor cells, we used the annexin V-FITC Apoptosis Detection Kit (BD Biosciences), followed by flow cytometry (FACS Vantage SE, Becton Dickinson). Within 24 h, 25% of the tumor cells were apoptotic, i.e., annexin V+ and To-Pro3+, and by 48 h, the % of annexin V+ and To-Pro3+ apoptotic tumor cells increased to 80% (data not shown).

In vivo delivery of dying tumor cells, DC maturation stimuli, and tumor protection assay. Different doses of irradiated J558-Ld− cells were injected i.v. into naïve BALB/c mice either alone or with αGalCer (2 or 0.2 μg/mouse) or 24 (2 or 0.2 μg/mouse) as a DC maturation stimulus. We also compared the glycolipids to agonistic anti-CD40 mAb (1C10, 25 μg, i.p.) or the Toll-like receptor ligands, poly IC (50 μg i.p., Invivogen). 14 d later, 5×10⁶ live J558 tumor cells were inoculated s.c. Tumor cell growth was measured with calipers every other day. Mice were scored positive for tumor, as soon as tumors became palpable and grew progressively. Mice were euthanized when tumor size exceeded 400 mm².

DC preparation from spleen. DCs were isolated from spleens using prior methods. In brief, splenocytes were released by homogenization followed by treatment with collagenase (collagenase D; Roche Diagnostics Corporation). A DC-enriched population was obtained using anti-CD11c coated magnetic beads (Miltenyi).

Serum cytokines. The serum concentrations of IL-1α, IL-1β, IL-2, IL-4, IL-5, IL-6, IL-10, IL-12 p70, TNF-α and IFN-γ were measured 2, 6, 12 and 24 hours after the injection of NKT cell ligands by Luminex (Upstate, N.Y.), according to the manufacturer's protocol. 25 μl of sample was preincubated with serum diluent and incubated for 2 h with Beadmates coated with anti-cytokine mAbs. The fluid was then aspirated and the wells incubated for 1.5 h with biotin-conjugated anti-cytokine mAbs followed by 30 min incubation with Beadlyte streptavidin-PE secondary antibody. Samples were acquired in duplicate by Luminex and analyzed using Beadview software (Upstate).

Cytokine production by DCs. After mice were given αGalCer or Compound 24 i.v., CD11c+ cells were isolated with anti-CD11c magnetic beads and cultured 4 h in the presence of Golgistop (from Cytofix/CytoPerm Plus kit; BD Biosciences). Cells were then stained for surface markers (CD11c and DX-5), permeabilized in 100 μl Cytofix/Cytoperm solution using manufacturer's instructions and stained for intracellular cytokines (IFN-γ and IL-12).

Stimulation of the mixed leukocyte reaction (MLR) by DCs. Spleen DCs were isolated using anti-CD11c magnetic beads 8 h after administration of αGalCer, 24, or PBS. Graded numbers of spleen DCs from BALB/C mice were irradiated and cultured with 2×10⁵ allogeneic C57B1/6 or syngeneic T cells, isolated using anti-CD4 coated magnetic beads (Miltenyi Biotech), in a 96-well flat-bottom plate for 88 h. During the final 16 h, ³H-thymidine (1 μCi/well) was added.

Flow cytometry. Acquisition of cell surface-activation markers was determined by flow cytometry. In brief, spleens were harvested 8 or 16 h following i.v. administration of αGalCer or 24, or i.p. administration of αCD40 or polyIC. Spleen CD11c+ DCs were isolated using anti-CD11c magnetic beads and stained with CD11c-FITC, CD8α− allophycocyanin, and PE-Conjugated I-Ad, CD40, CD80, CD86, CD119, B7-H1 and B7-DC. We used a FACSCalibur™ with data analysis in FlowJo (Tree Star).

CD1d-dimer binding assay. CD1d dimer (DimerX, BD Biosciences) was incubated overnight with 40 μM of each glycolipid at 32° C. and at neutral pH according to the manufacturer's protocol. The loaded CD1d dimers were incubated with a human NKT cell line at 4° C. for 60 min, followed by incubation with APC-conjugated secondary antibody. The cells were also co-stained with mAbs against the invariant NKT cell receptor (Vα24/Vβ11). At least 2×10⁵ lymphocyte-gated events were acquired to allow reliable estimation of NKT cells.

Human dendritic cell mediated NKT cell expansion. CD14+ monocytes were isolated from PBMC using anti-CD14 magnetic microbeads and cultured in the presence of GM-CSF and IL-4 to generate DCs, followed at day 5-6, by maturation in an inflammatory cytokine cocktail as described herein. To stimulate NKT cells, DCs were pulsed with α-GalCer and the tested analogs, and cultured with CD14− cells at a DC: responder ratio of 1:10. NKT cell expansion was monitored by flow cytometry based on the expression of the invariant NKT cell receptor (Vα24/Vβ11).

### Example 17

### Compound 24 Induces Upregulation of DC Surface Co-Stimulatory Molecules

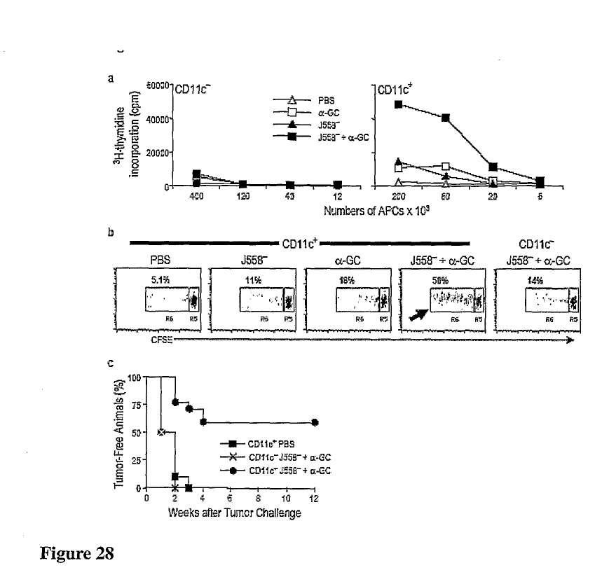

To determine whether αGalCer, and its analogues 24 and 27 (FIG. 28A) have a similar effect on DC maturation, we first looked for changes in the expression of several DC surface molecules, 15 hours following i.v. administration of glycolipid. We isolated spleen CD11c+ DCs and performed FACS analysis to evaluate the effect on the expression levels of the following markers (MHC class II, CD40, CD80, CD86, CD19, B7-H1, B7-DC) on CD11c+CD8+ DCs and CD11c+CD8− DCs. As shown in FIG. 28B, 24, was comparable to αGalCer in its ability to induce the upregulation of all these activation markers (all increased except for CD119, which decreased) on CD11c+CD8+ DCs. This included molecules involved in T cell costimulation (CD40, CD80, CD86, B7-DC and B7-H1), as well as antigen presentation (MHC class II). In contrast, 27 was much less effective, especially at the lower dose of 0.2 μg per mouse, and was therefore excluded from the experiments that follow below. In the case of CD11c+ CD8− DCs, 24 and αGalCer induced strong upregulation of CD86, B7-H1 and B7-DC but only slightly upregulated MHC II, CD40 and CD80 (see FIG. 28C). The responses to 24 and αGalCer, 15 h following i.v. administration, paralleled and exceeded those seen with other known stimuli for DC maturation in vivo, i.e. agonistic αCD40 mAb and TLR3 ligand, polyIC (FIG. 28D), although we found that polyIC changes the DC phenotype much more rapidly than NKT activating glycolipids (our unpublished data). When different doses of 24 and αGalCer were examined (2, 0.2, 0.02 and 0.002 μg/mouse), the degree of DC maturation correlated with the dose of glycolipid administered (data not shown). Therefore, αGalCer and 24, but not 27, act as rapid and efficient inducers of splenic DC maturation in vivo, as determined by the expression of surface activation markers, and are comparable in efficacy to other stimuli. However, unlike other maturation stimuli, αGalCer and 24 did not have a direct effect on DCs, since they were unable to directly stimulate DC maturation from bone marrow progenitors in culture (data not shown).

### Example 18

### αGalcer and Compound 24 Induce Rapid DC Maturation Following Activation of NKT Cells

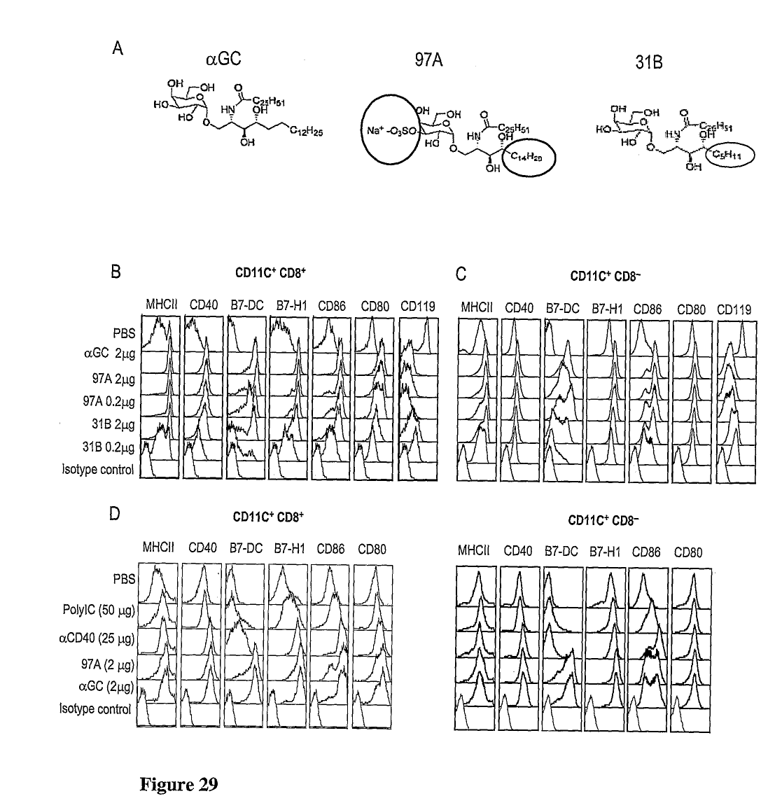

NKT cells respond quickly to the presentation of αGalCer on CD1d molecules. To address the role of NKT cells in the rapid maturation of DCs observed upon i.v. administration of αGalCer or 24, we tested mice lacking these T cells because of the deletion of the essential TCR Jα28i sequences. The DCs from Jα281−/− mice (also known as Jα18−/−) did not mature in response to αGalCer or 24 in vivo, but expressed comparable levels of CD86, CD80 (FIG. 29A) and other maturation markers (CD40, CD80 and MHC Class II) (data not shown) to wild type mice (C57B1/6) when given the PBS control or when responding to αCD40 mAb (FIG. 29A). Furthermore, Jα281−/− mice lacked the ability to release IFN-γ and IL12p70 into the serum, following i.v. injection of αGalCer or 24, as typically occurs in wild type mice (FIG. 29B). Thus αGalCer and 24 rapidly mature DCs in situ, as assessed by the surface markers of DCs in the spleen and cytokine release into the serum, through NKT cell activation.

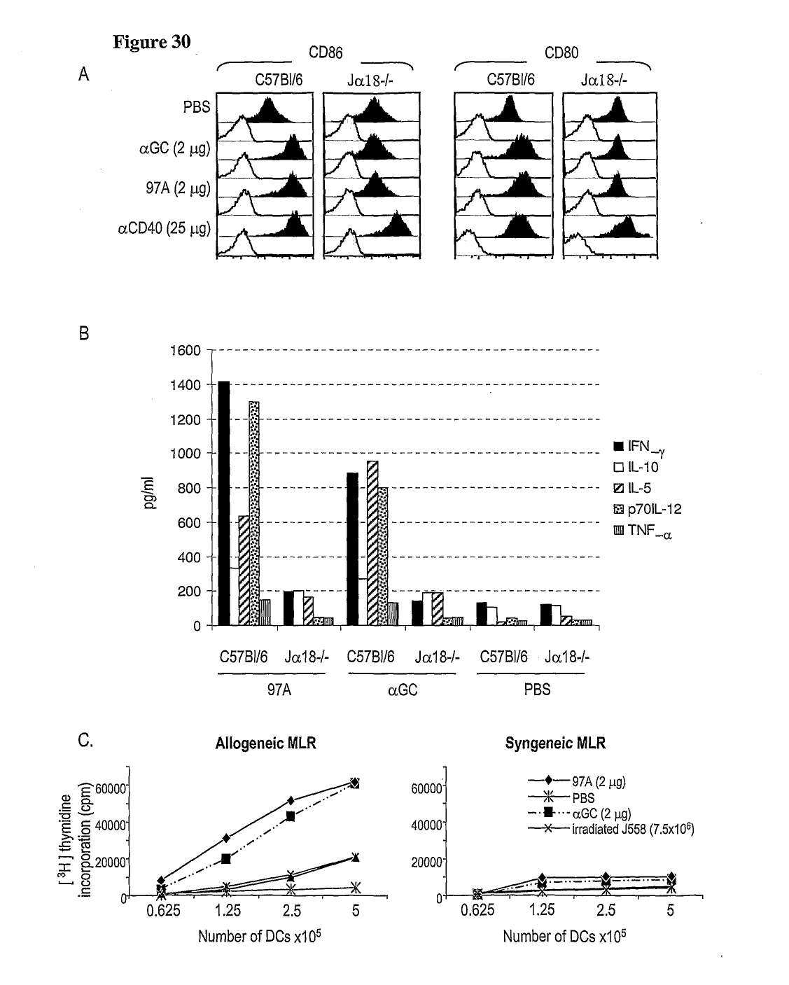

To evaluate maturation of DCs from αGalCer or 24 treated mice with a functional assay, we isolated DCs with αCD11c magnetic beads and tested them as stimulators for allogeneic (spleen CD4+ T cells isolated from naïve C57B1/6) or syngeneic (spleen CD4+ T cells isolated from naïve BALB/C) T cells in the primary mixed lymphocyte reaction (MLR). As shown in FIG. 29C (right panel), DCs from all groups of mice (controls and mice treated with αGalCer or 97A) showed no stimulatory activity in the syn-MLR at the DC doses we tested. DCs from control mice were also incapable of stimulating allogeneic T cell proliferation (FIG. 30C, left panel), confirming that most DCs in the spleen are functionally immature. In contrast, DCs from αGalCer or 24 treated mice were equally potent stimulators of the allo-MLR (FIG. 29C, left panel).

### Example 19

### Compound 24 Induces the Release of Cytokines into the Serum

To document the innate response to glycolipid, we used a Luminex assay to assess the kinetics of cytokine release in the serum of mice injected i.v. with αGalCer or 24. Both glycolipids induced rapid increases in serum IFN-γ, IL-12p70, IL-4, TNF-α and IL-2 concentrations; however the responses to 24 were significantly higher at all time points examined, particularly in the case of serum IL-12p70. Immunization of mice with 24 induced ˜1000 pg/ml of serum IL-12p70 by 6 h, which declined to ˜900 pg/ml by 12 h. In contrast, the serum concentration of IL-12p70 in mice stimulated in vivo with αGalCer, was ˜600 pg/ml by 6 h and declined to ˜200 pg/ml by 12 h see middle panel of top row in FIG. 3A). However in the case of serum concentrations of IFN-γ, 97A had slower kinetics than αGalCer, inducing a maximum release of ˜3000 pg/ml by 12 h. αGalCer on the other hand, induced a higher and more rapid response (6000 pg/ml by 6 h), which declined by 12 hours to ˜1200 pg/ml (see first panel of top row in FIG. 30A).

We also performed a dose-response study in which we compared different doses of αGalCer and 24 for their efficiency to induce cytokine release into the serum, 15 hours following i.v. injection. Confirming the previous results, production of both IFN-γ and IL-12p70 was significantly (at least 2.5-fold) higher in mice treated with 24 vs. αGalCer for all doses tested (FIG. 30B). Furthermore, unlike αGalCer, 24 was able to produce significant amounts of cytokines even at the lowest dose tested (0.02 μg/mouse). IFNγ in the serum of mice stimulated with 0.02 μg of 24 for 15 hours was ˜1200 pg/ml whereas in the serum of mice stimulated with the same dose of αGalCer, IFN-γ was only ˜100 pg/ml. In addition, the concentration of IL-12p70 in the serum of mice stimulated with 0.02 μg of 97A for 15 h was ˜900 pg/ml but only ˜50 pg/ml for mice stimulated with the same dose of αGalCer. Therefore, 24 is more effective than αGalCer in inducing cytokine release into the serum following in vivo administration.

### Example 20

### Compound 24 Induces Cytokine Release by DCs, when Compared to αGalCer

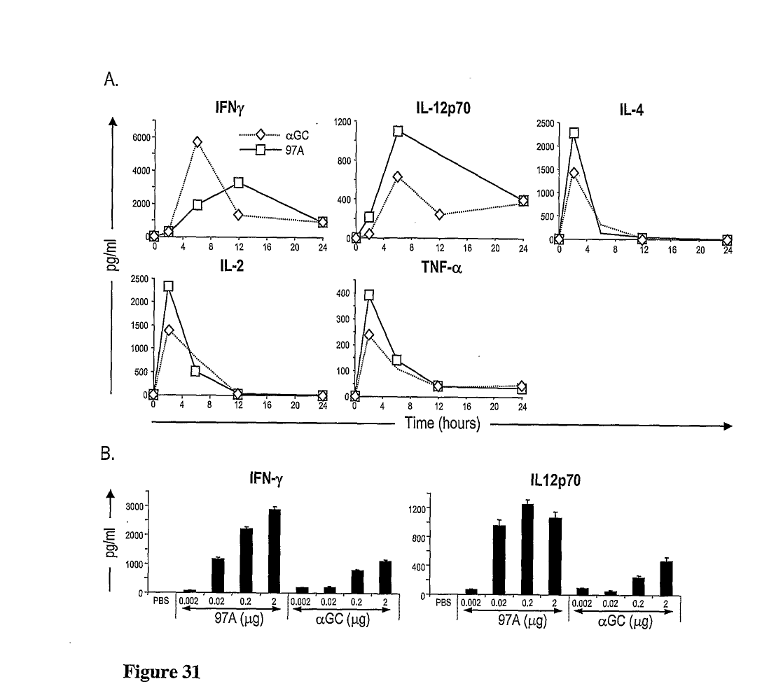

To demonstrate that 24 and αGalCer were in fact priming DCs in vivo causing them to produce large amounts of IL-12p70 and IFN, DCs were isolated from mice stimulated 2 h, 6 h and 12 h in vivo with either αGalCer or 24 (2 μg or 0.2 μg). Spleen CD11c+ enriched DCs were prepared, which also contained a small fraction of NK cells, and the cells were cultured for 4 hours in the presence of BFA (1 μg/ml). IFN-γ and IL-12 production by CD11c+DX5− DCs as well as CD11c+DX5+ NK cells was determined by intracellular staining. As shown in FIG. 31 and confirming the previous results obtained using mice sera, production of IFN-γ by CD11c+DX5+ NK cells and production of IL-12 by CD11c+DX5− DCs were significantly higher in mice treated with 24 vs. αGalCer. DCs from αGalCer or 24-primed mice cultured in the presence of αCD40 produced significant but comparable amounts of IL-12p40, IL-6 and TNF-α, in contrast to DCs from control mice (by ELISA and Luminex assays) (data not shown). IL-4 and IL-10 (<10 pg/ml) could not be detected by ELISA, in the culture supernatants of DCs from αGalCer or 24 primed mice (data not shown). Thus, even though αGalCer and 24 are comparable in their ability to induce phenotypic maturation of DCs, 24 is superior to αGalCer in inducing functional maturation of DCs, as assessed by cytokine production in both serum and in vitro DC cultures, in the presence of αCD40.

### Example 21

### Compound 24 Induces Long Lived, Prophylactic Tumor Immunity when Administered with Irradiated Tumor Cells

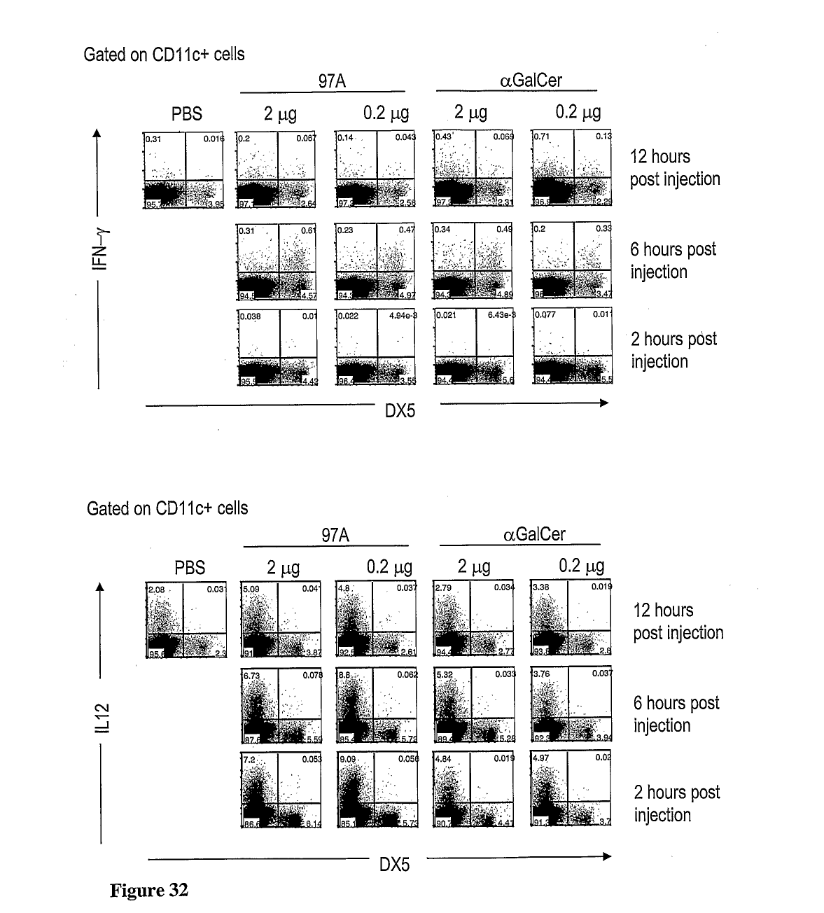

In order to determine whether 24 was similar to αGalCer in its ability to induce protective tumor immunity in vivo, the compound was co-administered with irradiated J558Ld− tumor cells and tumor suppression was assessed. Naïve BALB/C mice were immunized with PBS, αGalCer or 24 alone (2 or 0.2 μg/mouse) or in the presence of 1×10⁷ irradiated J558Ld− tumor cells. 14 days after the vaccination, the mice were challenged with 5×10⁶ J558 live tumor cells s.c. It is known from prior research that NKT cells serve as adjuvants to allow tumor-capturing DCs to induce strong and combined CD4+ and CD8+ T cell immunity. The tumor grew progressively within 7-10 days in mice that had received either PBS, αGalCer or 24 alone or irradiated J558Ld− tumor cells alone (FIG. 32A). Injection of αGalCer or 24 in the presence of irradiated J558Ld− tumor cells, at both the high (2 μg/mouse) and the low dose (0.2 μg/mouse), fully protected mice from the J558 tumor challenge (100% protection in 3 experiments). Thus tumor immunity was elicited by a single i.v. vaccination with either αGalCer or 24 in the presence of irradiated J558Ld− tumor cells.

### Example 22

### The Combination of αCD40 and PolyIC Mimics Compound 24 in Inducing Protective Immunity

In order to compare 24 with other DC maturation stimuli, agonistic αCD40 monoclonal antibody as well as the TLR3 ligand, poly IC were used. The glycolipids were the only adjuvant that could independently induce protective immunity to irradiated tumor injected i.v., but the combined activation by αCD40 and poly IC was also effective (FIG. 32B). Interestingly, and as indicated in FIG. 30B, each stimulus (poly IC, αCD40, αGalCer, 24) induced similar phenotypic changes of maturation, i.e., increased expression of CD40, CD86, MHC class II, B7-H1, B7-DC, and decreased interferon-γ receptor or CD119, but for protective immunity, either αGalCer or 24 or the combination of poly IC and αCD40 was required. Therefore to elicit protective immunity to a syngeneic tumor, a synthetic glycolipid, such as 24 or the combination of a proinflammatory TLR ligand, poly IC, and agonistic αCD40 antibody are able to mimic the effects of αGalCer.

### Example 23

### Compound 24 Binds to Human CD1d Molecule and Efficiently Expands NKT Cells

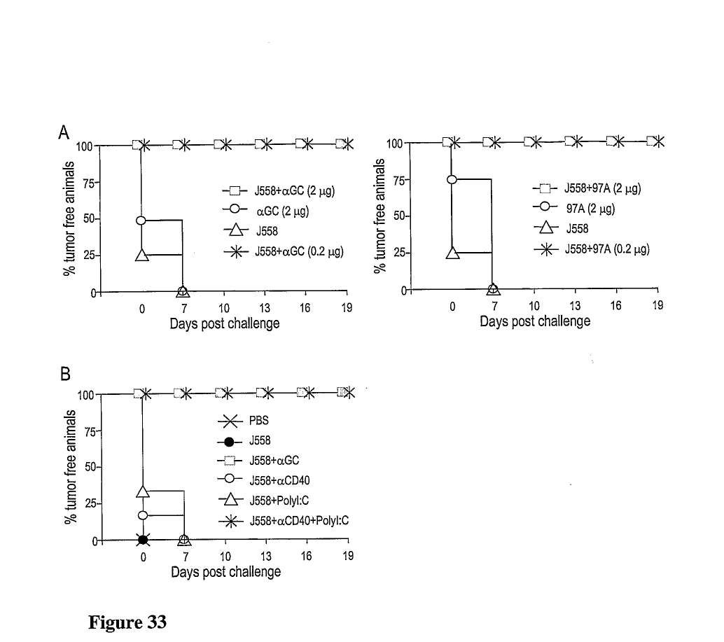

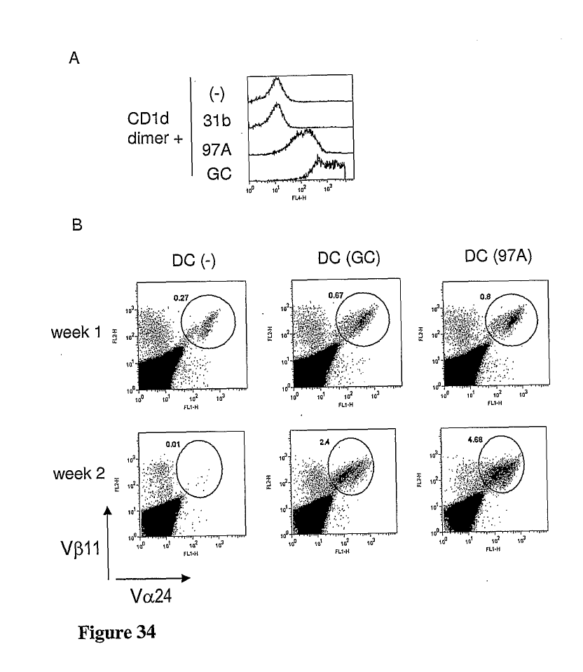

In order to determine whether some of the findings from the animal experiments could be translated to the biology of human NKT cells, CD1d dimers loaded with 24 were assessed for their ability to efficiently bind Vα24/Vβ11 invariant TCR expressing NKT cells, which was the case, although to a lesser extent than α-GalCer, whereas dimers loaded with 27 did not bind to invariant TCR receptors on human NKT cells (FIG. 33A). Human monocyte derived DCs loaded with αGalCer were efficient inducers of NKT cell expansion in culture. In accordance with the CD1d dimer binding experiments, mature DCs loaded with 24 efficiently expanded human NKT cells. The extent of expansion was comparable or higher than expansion mediated by DCs loaded with αGalCer (FIG. 33B and Table 1). 27 did not lead to any NKT cell expansion because of the absence of CD1d binding. Stimulation of NKT cells with DCs loaded with 24 or αGalCer also induced rapid production of IFNγ, IL-13 and IL-2 (data not shown).

TABLE 1

| 1.1. Donor | DC alone | DC + αGalCer | DC + 97A |
|---|---|---|---|
| 1. | 0.72 | 3.55 | 3.19 |
| 2. | 0.03 | 2.65 | 5.95 |
| 3. | 0.03 | 1.53 | 1.51 |
| 4. | 0.05 | 3.55 | 4.95 |

It will be appreciated by a person skilled in the art that the present invention is not limited by what has been particularly shown and described hereinabove. Rather, the scope of the invention is defined by the claims that follow:

What is claimed is:

1-131. (canceled)

132. A method for treating, delaying onset of, reducing incidence of, suppressing or reducing the severity of neoplasia in a subject, comprising the steps of:
a. culturing immature dendritic cells with a neoplastic cell;
b. contacting the culture in (a) with a compound characterized by the structure of the formula 1 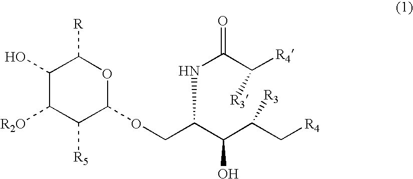
wherein,
R═COOR₁ or CH₂OR₁;
R₁═H or an alkyl group;
R₂═H or SO₃⁻;
R₃═H or OH;
R₃′═H or OH;
R₄═H, unsaturated or saturated, alkyl group;
R₄′═H, unsaturated or saturated, alkyl group; and
R₅═OH, acetamide or a halogen atom;
or a pharmaceutically acceptable salt thereof, wherein if R═CH₂OR₁, R₂═H, R₃ is OH and R₃′ is H, then R₅=acetamide, halogen atom or OH in an axial position or R₄═H, unsaturated or saturated, alkyl chain having 9 carbon atoms or fewer, or R₄′═H, unsaturated or saturated, alkyl chain having 20 carbon atoms or fewer; and
c. administering the culture in (b) to said subject.

133. The method of claim 132, wherein said compound is represented by the structure of formula 2: 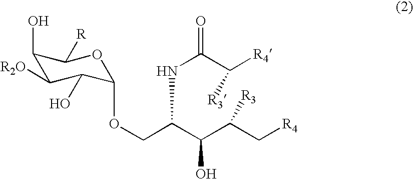
wherein
R═COOR₁ or CH₂OR₁;
R₁═H or an alkyl group;
R₂═H or SO₃⁻;
R₃═H or OH;
R₃′═H or OH; and
R₄═H, unsaturated or saturated, alkyl group; and
R₄′═H, unsaturated or saturated, alkyl group;
or a pharmaceutically acceptable salt thereof, wherein if R═CH₂OR₁, R₂═H, R₃ is OH and R₃′ is H, then R₄═H, unsaturated or saturated, alkyl chain having 9 carbon atoms or fewer, or R₄′═H, unsaturated or saturated, alkyl chain having 20 carbon atoms or fewer.

134. The method of claim 133, wherein said compound is represented by the structure of formula 3: 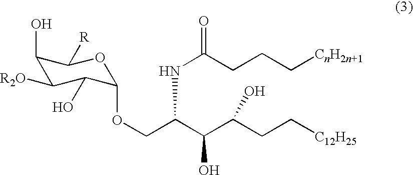
wherein,
R═COOR₁ or CH₂OR₁
R₁═H or an alkyl group;
R₂═SO₃⁻;
n=integer;
or a pharmaceutically acceptable salt thereof.

135. The method of claim 133, wherein said compound is selected from the group consisting of 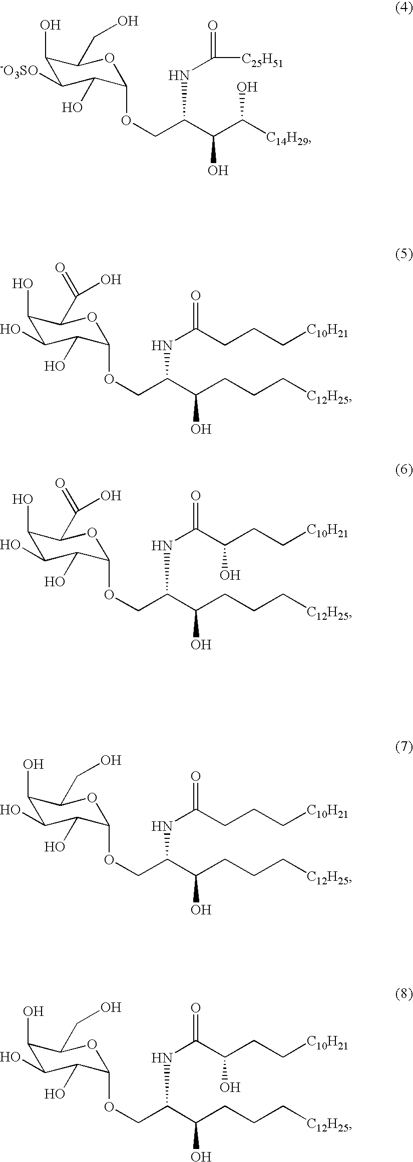
and a respective pharmaceutically acceptable salt thereof.

136. The method of claim 132, wherein said compound is represented by the structure of formula 9: 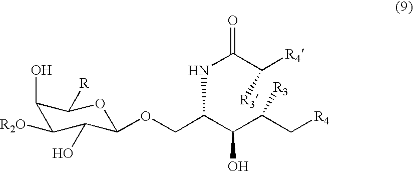
wherein
R═COOR₁ or CH₂OR₁;
R₁═H or an alkyl group;
R₂═H or SO₃⁻;
R₃═OH;
R₃′═H or OH; and
R₄═H, unsaturated or saturated, alkyl group; and
R₄′═H, unsaturated or saturated, alkyl group;
or a pharmaceutically acceptable salt thereof, wherein if R═CH₂OR₁, R₂═H and R₃′ is H, then R₄═H, unsaturated or saturated, alkyl chain having 9 carbon atoms or fewer, or R₄′═H, unsaturated or saturated, alkyl chain having 20 carbon atoms or fewer.

137. The method of claim 136, wherein said compound is represented by the structure of formula 10: 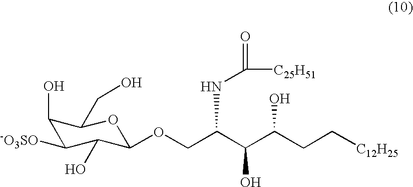
or a pharmaceutically acceptable salt thereof.

138. The method of claim 132, wherein said compound is a ligand for an NKT (natural killer T) cell and is in a complex with a CD1 molecule.

139. The method of claim 132, wherein said neoplastic cell is irradiated.

140. The method of claim 132, wherein said compound is at a concentration ranging from 1-1000 ng/ml.

141. The method of claim 132, wherein said culture in step (c) comprises dendritic cells with MHC II^{hi}, CD80^{hi}, CD86^{hi}, B7H1^{hi}, B7-DC^{hi}, or a combination thereof.

142. The method of claim 132, wherein said culture further comprises NKT cells.

143. A method for treating, delaying onset of, reducing incidence of, suppressing or reducing the severity of neoplasia in a subject, comprising the steps of:
a. culturing immature dendritic cells with a neoplastic cell;
b. contacting the culture in (a) with a compound characterized by the structure of the formula 11: 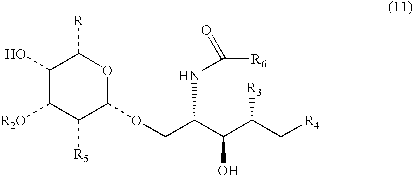
wherein,
R═COOR₁ or CH₂OR₁;
R₁═H or an alkyl group;
R₂═H or SO₃⁻;
R₃═H or OH;
R₄═H, unsaturated or saturated, alkyl group;
R₅═OH, acetamide or a halogen atom; and
R₆═X-A
A=
dialkyl phenyl; 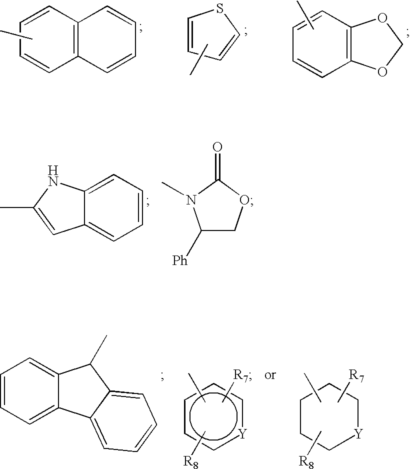
X=alkyl, alkenyl, alkoxy, thioalkoxy, substituted furan, or unsubstituted furan;
Y═N or C
R₇=halogen, H, phenyl, alkyl, alkoxy, nitro or CF₃; and
R₈=methyl or H;
or a pharmaceutically acceptable salt thereof; and
c. administering the culture in (b) to said subject.

144. The method of claim 143, wherein said compound is represented by the structure of formula 12: 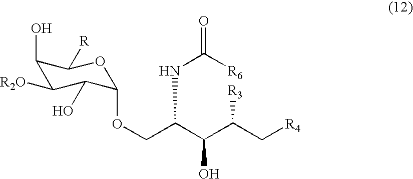
or a pharmaceutically acceptable salt thereof.

145. The method of claim 144, wherein said compound is represented by the structure of formula 13: 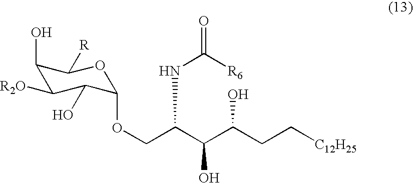
or a pharmaceutically acceptable salt thereof.

146. The method of claim 143, wherein said compound is selected from the group consisting of 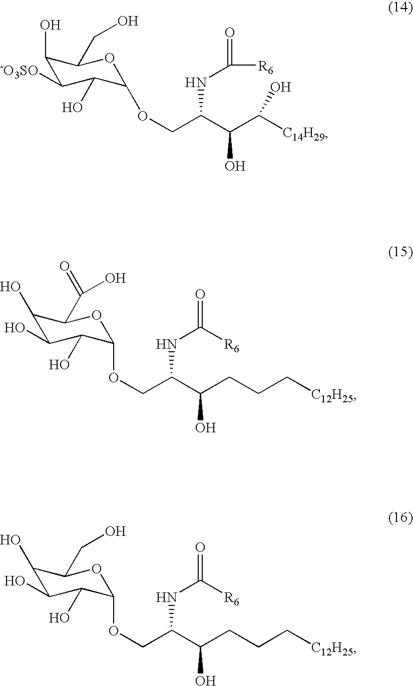
and a respective pharmaceutically acceptable salt thereof.

147. The method of claim 143, wherein said compound is a ligand for an NKT (natural killer T) cell and is in a complex with a CD1 molecule.

148. The method of claim 143, wherein said neoplastic cell is irradiated.

149. The method of claim 143, wherein said compound is at a concentration ranging from 1-1000 ng/ml.

150. The method of claim 143, wherein said culture in step (c) comprises dendritic cells with MHC II^{hi}, CD80^{hi}, CD86^{hi}, B7-H1^{hi}, B7-DC^{hi}, or a combination thereof.

151. The method of claim 143, wherein said culture further comprises NKT cells.

152. A method for treating, delaying onset of, reducing incidence of, suppressing or reducing the severity of neoplasia in a subject, comprising the step of administering to said subject a composition comprising a neoplastic cell and a compound characterized by the structure of the formula 1: 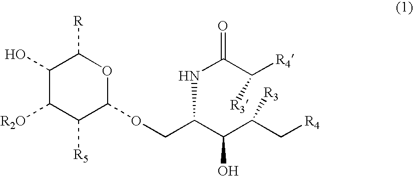
wherein,
R═COOR₁ or CH₂OR₁;
R₁—H or an alkyl group;
R₂═H or SO₃⁻;
R₃H or OH;
R₃′═H or OH;
R₄═H, unsaturated or saturated, alkyl group;
R₄′═H, unsaturated or saturated, alkyl group; and
R₅═OH, acetamide or a halogen atom;
or a pharmaceutically acceptable salt thereof, wherein if R═CH₂OR₁, R₂═H, R₃ is OH and R₃′ is H, then R₅=acetamide, halogen atom or OH in an axial position or R₄═H, unsaturated or saturated, alkyl chain having 9 carbon atoms or fewer, or R₄′═H, unsaturated or saturated, alkyl chain having 20 carbon atoms or fewer.

153. The method of claim 152, wherein said compound is represented by the structure of formula 2: 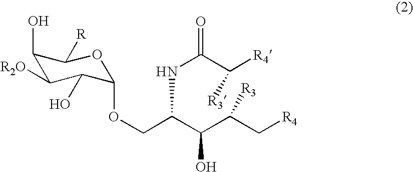
wherein
R═COOR₁ or CH₂OR₁;
R₁═H or an alkyl group;
R₂═H or SO₃⁻;
R₃═H or OH;
R₃′═H or OH; and
R₄═H, unsaturated or saturated, alkyl group; and
R₄′═H, unsaturated or saturated, alkyl group;
or a pharmaceutically acceptable salt thereof, wherein if R═CH₂OR₁, R₂═H, R₃ is OH and R₃′ is H, then R₄═H, unsaturated or saturated, alkyl chain having 9 carbon atoms or fewer, or R₄′═H, unsaturated or saturated, alkyl chain having 20 carbon atoms or fewer.

154. The method of claim 153, wherein said compound is represented by the structure of formula 3: 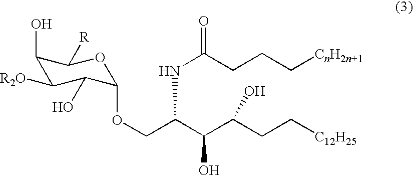
wherein,
R═COOR₁ or CH₂OR₁
R₁═H or an alkyl group;
R₂═SO₃⁻;
n=integer;
or a pharmaceutically acceptable salt thereof.

155. The method of claim 153, wherein said compound is selected from the group consisting of 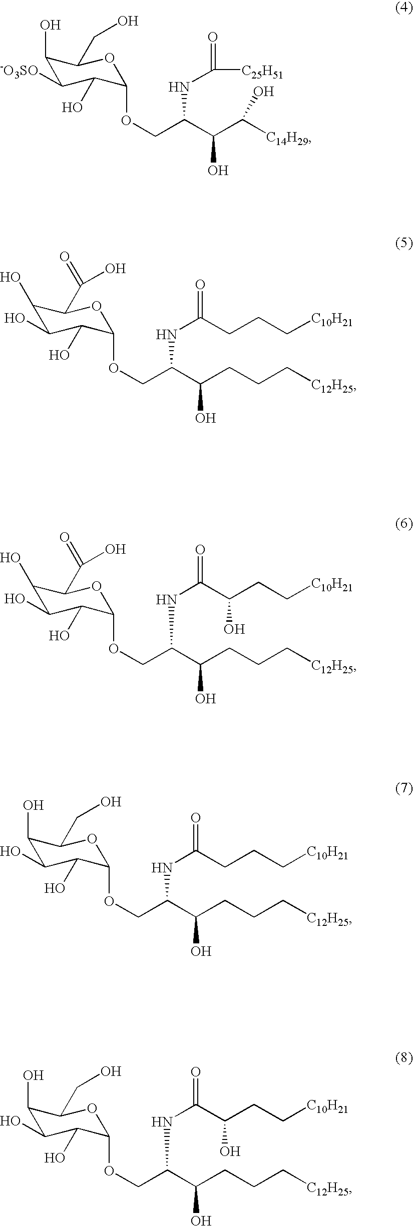
and a respective pharmaceutically acceptable salt thereof.

156. The method of claim 152, wherein said compound is represented by the structure of formula 9: 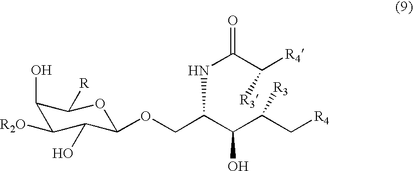
wherein
R═COOR₁ or CH₂OR₁;
R₂═H or an alkyl group;
R₃═H or SO₃⁻;
R₃═OH;
R₃′═H or OH; and
R₄═H, unsaturated or saturated, alkyl group; and
R₄′═H, unsaturated or saturated, alkyl group;
or a pharmaceutically acceptable salt thereof, wherein if R═CH₂OR₁, R₂═H and R₃′ is H, then R₄═H, unsaturated or saturated, alkyl chain having 9 carbon atoms or fewer, or R₄′═H, unsaturated or saturated, alkyl chain having 20 carbon atoms or fewer.

157. The method of claim 156, wherein said compound is represented by the structure of formula 10: 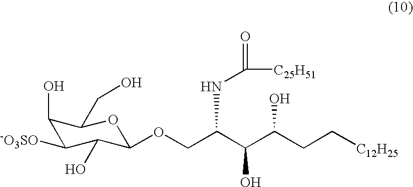
or a pharmaceutically acceptable salt thereof.

158. The method of claim 152, wherein said neoplastic cell is irradiated.

159. The method of claim 152, wherein said compound is at a concentration ranging from 1-1000 ng/ml.

160. The method of claim 152, further comprising the step of administering said composition repeatedly.

161. The method of claim 152, wherein said composition further comprises a cytokine.

162. The method of claim 152, wherein said composition further comprises an NKT cell.

163. A method for treating, delaying onset of, reducing incidence of, suppressing or reducing the severity of neoplasia in a subject, comprising the step of administering to said subject a composition comprising a neoplastic cell and a compound characterized by the structure of the formula 11: 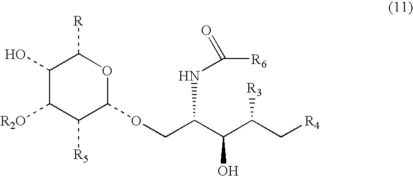
wherein,
R═COOR₁ or CH₂OR₁;
R₁═H or an alkyl group;
R₂═H or SO₃⁻;
R₃═H or OH;
R₄═H, unsaturated or saturated, alkyl group;
R₅═OH, acetamide or a halogen atom; and
R₆═X-A
A=
dialkyl phenyl; 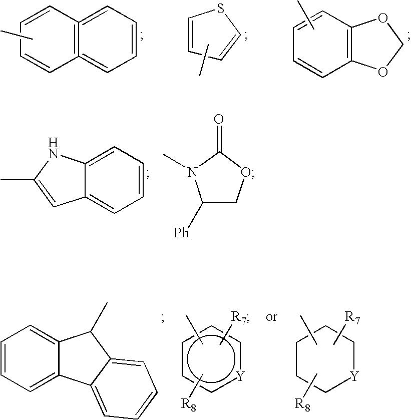
X=alkyl, alkenyl, alkoxy, thioalkoxy, substituted furan, or unsubstituted furan;
Y═N or C
R₇=halogen, H, phenyl, alkyl, alkoxy, nitro or
CF₃; and
R₈=methyl or H;
a. or a pharmaceutically acceptable salt thereof.

164. The method of claim 163, wherein said compound is represented by the structure of formula 12: 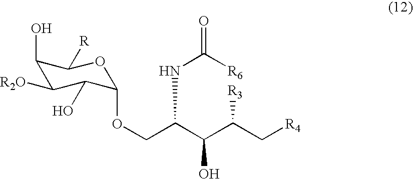
or a pharmaceutically acceptable salt thereof.

165. The method of claim 164, wherein said compound is represented by the structure of formula 13: 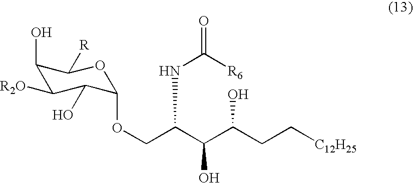
or a pharmaceutically acceptable salt thereof.

166. The method of claim 164, wherein said compound is selected from the group consisting of 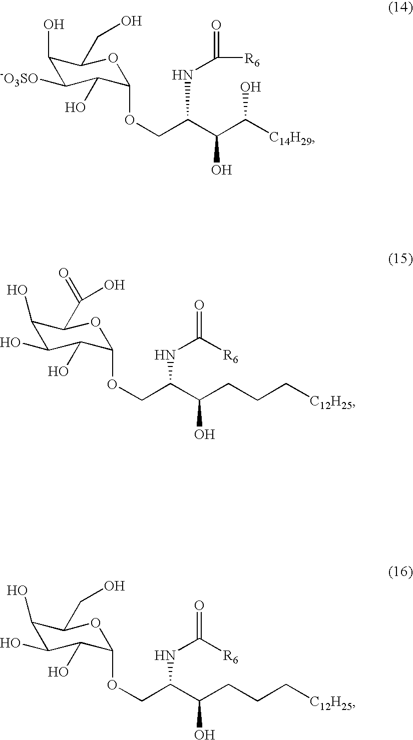
and a respective pharmaceutically acceptable salt thereof.

167. The method of claim 164, wherein said compound is at a concentration ranging from 1-1000 ng/ml.

168. The method of claim 164, further comprising the step of administering said composition repeatedly.

169. The method of claim 164, wherein said composition further comprises a cytokine.

170. The method of claim 164, wherein said composition further comprises an NKT cell.
